SEQUENCE LISTING (183 sequences)

SEQ ID NO: 1 - DNA, 18 bp, Artificial
  Synthetic
taatacgactcactatag

SEQ ID NO: 2 - DNA, 18 bp, Artificial
  Synthetic
aattaaccctcactaaag

SEQ ID NO: 3 - DNA, 1320 bp, Artificial
  Synthetic
ccaaggtcctgcttttgcatcttaagccgcccctcctttctccaacagacacgaggagcaaagggtaactgagagggagtagcaggtaaagcccacagtgttctcaccgggtcaccctgaggacttcttagttataggagctgcttcattctctccgatccgtgctggcttctctcccactctcacttgaaggaaggggaaagctttctaagtttagccgtcactctggaatttaacatcatcgatgttctactgtgcagcgttgatggttcgatgggctctctccagggaggacggaaatccagatgccacttccttcttcatttacatagcattcatatcacgtcgcgactgacgctcaggaatgagtcatcctgtgtccctgcaggtggccgtgggcacacctgaggaagcaaagtccggcacgcagctggcagcagccatcgccgcaacataagctcccgaggaaggagtccagagacacagagagcaagatgagttccgagacaggccctgtagctgttgatcccactctgaggagaagaattgagccccacgagtttgaagtcttctttgacccccgggaacttcggaaagagacctgtctgctgtatgagatcaactggggaggaaggcacagcatctggcgacacacgagccaaaacaccaacaaacacgttgaagtcaatttcatagaaaaatttactacagaaagatacttttgtccaaacaccagatgctccattacctggttcctgtcctggagtccctgtggggagtgctccagggccattacagaatttttgagccgatacccccatgtaactctgtttatttatatagcacggctttatcaccacgcagatcctcgaaatcggcaaggactcagggaccttattagcagcggtgttactatccagatcatgacggagcaagagtctggctactgctggaggaattttgtcaactactccccttcgaatgaagctcattggccaaggtacccccatctgtgggtgaggctgtacgtactggaactctactgcatcattttaggacttccaccctgtttaaatattttaagaagaaaacaacctcaactcacgtttttcacgattgctcttcaaagctgccattaccaaaggctaccaccccacatcctgtgggccacagggttgaaatgacttctgggagttggggatggatgaaatgactccttgtatgtcttgacagcaagcattgattacccactaaagagcgactgccacaaggaaaaaaaaaaaaaaaaaaaaaaaaaaaaaaaaaaaaaaaaaaaaaaaa

SEQ ID NO: 4 - protein, 229 aa, Artificial [sequence omitted]

SEQ ID NO: 5 - DNA, 809 bp, Artificial
  Synthetic
cccgctgctctgctgcctgcccggggtaccaacatggcccagaagcgtcctgcctgcaccctgaagcctgagtgtgtccagcagctgctggtttgctcccaggaggccaagaagtcagcctactgcccctacagtcactttcctgtgggggctgccctgctcacccaggaggggagaatcttcaaagggtgcaacatagaaaatgcctgctacccgctgggcatctgtgctgaacggaccgctatccagaaggccgtctcagaagggtacaaggatttcagggcaattgctatcgccagtgacatgcaagatgattttatctctccatgtggggcctgcaggcaagtcatgagagagtttggcaccaactggcccgtgtacatgaccaagccggatggtacgtatattgtcatgacggtccaggagctgctgccctcctcctttgggcctgaggacctgcagaagacccagtgacagccagagaatgcccactgcctgtaacagccacctggagaacttcataaagatgtctcacagccctggggacacctgcccagtgggccccagccctacagggactgggcaaagatgatgtttccagattacactccagcctgagtcagcacccctcctagcaacctgccttgggacttagaacaccgccgccccctgccccacctttcctttccttcctgtgggccctctttcaaagtccagcctagtctggactgcttccccatcagccttcccaaggttctatcctgttccgagcaacttttctaattataaacatcacagaacatcctgga

SEQ ID NO: 6 - protein, 146 aa, Artificial [sequence omitted]

SEQ ID NO: 7 - protein, 167 aa, Artificial
  Synthetic
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 8 - DNA, 504 bp, Artificial
  Synthetic
ttgtctgaagtcgaatttagccacgaatactggatgcgtcacgcgctgacgctggcgaaacgtgcctgggatgagcgggaagtgccggtcggcgcggtattagtgcataacaatcgggtaatcggcgaaggctggaaccgcccgattggtcgccatgatcccaccgcacatgcagaaatcatggccctgcggcagggtggtctggtgatgcaaaattatcgtctgatcgacgccacgttgtatgtcacgcttgaaccatgtgtaatgtgtgccggagcgatgatccacagtcgcattggtcgcgtggtctttggtgcgcgtgacgcgaaaactggcgctgcgggatctttaatggatgtgctgcatcatccgggtatgaatcaccgagtggaaattacggaaggaatactggcggatgagtgcgcggcgttgctcagtgacttctttcgcatgcgccgccaggaaattaaagcgcagaaaaaagcgcaatcctcgacggattaa

SEQ ID NO: 9 - DNA, 6902 bp, Artificial
  Synthetic
gaggcgctgaggcggccgtggcggcggcggcggcggcggcggcagcggcggccaagcggccaggttggcggccggggctccgggccgcgcgaggccacggccacgccgcgccgctgcgcacaaccaacgaggcagagcgccgcccggcgcgagactgcggccgaagcgtggggcgcgcgtgcggaggaccaggcgcggcgcggctgcggctgagagtggagcctttcaggctggcatggagagcttaaggggcaactgaaggagacacactggccaagcgcggagttctgcttacttcagtcctgctgagatactctctcagtccgctcgcaccgaaggaagctgccttgggatcagagcagacataaagctagaaaaatttcaagacagaaacagtctccgccagtcaagaaaccctcaaaagtattttgccatggatatagaagatgaagaaaacatgagttccagcagcactgatgtgaaggaaaaccgcaatctggacaacgtgtcccccaaggatggcagcacacctgggcctggcgagggctctcagctctccaatgggggtggtggtggccccggcagaaagcggcccctggaggagggcagcaatggccactccaagtaccgcctgaagaaaaggaggaaaacaccagggcccgtcctccccaagaacgccctgatgcagctgaatgagatcaagcctggtttgcagtacacactcctgtcccagactgggcccgtgcacgcgcctttgtttgtcatgtctgtggaggtgaatggccaggtttttgagggctctggtcccacaaagaaaaaggcaaaactccatgctgctgagaaggccttgaggtctttcgttcagtttcctaatgcctctgaggcccacctggccatggggaggaccctgtctgtcaacacggacttcacatctgaccaggccgacttccctgacacgctcttcaatggttttgaaactcctgacaaggcggagcctcccttttacgtgggctccaatggggatgactccttcagttccagcggggacctcagcttgtctgcttccccggtgcctgccagcctagcccagcctcctctccctgtcttaccaccattcccacccccgagtgggaagaatcccgtgatgatcttgaacgaactgcgcccaggactcaagtatgacttcctctccgagagcggggagagccatgccaagagcttcgtcatgtctgtggtcgtggatggtcagttctttgaaggctcggggagaaacaagaagcttgccaaggcccgggctgcgcagtctgccctggccgccatttttaacttgcacttggatcagacgccatctcgccagcctattcccagtgagggtcttcagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtgaggggacgattccagtgcgctccaatgcgagcatccaaacgtgggacggggtgctgcaaggggagcggctgctcaccatgtcctgcagtgacaagattgcacgctggaacgtggtgggcatccagggatccctgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgccctgacccgggcagacatgatggggggtgcagggggctgtgggcatccagcgtcatcctccagaacctcacatctgaactgggggcaggtgcataccttggggagggagtagggggacacgggggaccaccaggtgtccacggttgtccccagcatctcacatcagacctggggcaggtgcgcagtgtggggaggggatggggtgcgtcagggcccagcatcgccgcctggcatctctctgccgcagcatttccccttctgaaccgtccagtgactgctttcaatctcggtttacgtttagaaattgagttctactgagtagggcttccttaagtttaggaaaatagaaattactttgtgtgaaattcttgaataaataatttattcagagctaggaatgtggtttataaaataggaagtaattgtgtcaggtcacttttatgccacattattttaattgcaaaaaagcatctatatatggaggagggtgggaaaatagaggtaggaaatagtagcctaaaggaaatcgccacacgtctgtctaaacttaggtctcttttctccgtaggtacctccctgggtagttccacacactaggttgtaacagtctctccctgaggagcagactcccagcatggtgtagcgtggccctgtcatgcacatggggtcccgcagcagtgactgtgtgtcctgcagaggcgtgacccaggcccctgtagccctcagcctcctctagaagcttctgtactccttgtaggatcagatcatggaaaacttttctcagtttacttctaagtaatcacagataatacatggccagtaatcccaggctggccattcattcaggttttttaaaggatatttaacttttatggactagaaggaatcacgagggctactgcacaatacatggcctaagttccctctgttccttcctctgaatcgaatggatgtgggtgaccgcccgaaggccttcacaggatggaagtagaatgatttcagtagatactcattcttggaaaatgccatagttttaaattattgtttccagctttatcaaagacatgtttgaaaaataaaaagcatccaagtgagagctggtgagaccacgtgctgctggcgtagtgtaggccagacattgacagtcctgacgggagctcagggctgcccagcgcccagcgtgcacgggacggccccacgacagagggagtcagcccgggaggtcaggagcgcggcgggcgagggccctgtgtggaccacctccaccaagctcagagatttgcaccaggtgccttgttgcctccgctcaggatgaaagaggagctgagagaagtgctctgcctgccagtgcagtgcccagctccaaggctctagagggtgttcaggtgggtctcctggggccatggggagagattggtgcagaccttaccccacagcatacacctgccacagcgaaatccagggtgttggcacctgtgtgtccgtgatgagcctaggaaaccagagcaggggcagaggggcgtcatcctcccaccggacgctgggagctcagaccccaaaactgaaacaccgtggcttcggcggggggtgtgcctcctgatgtcaggagccccatccacgtgtgtccacacagatctcgtcgcagcacggcaggaaggggtgctgcttagggctcattgttggggacatgaccgggttcagcggctagaacatctgccccacagcagcctcctcctccaccgaagagggtagttgtctccctgaagcagtcacagcaggcgtctctgccgctccgtcaccacagtggggttttgttcaggcagatcgcgctggggttctgcacctgcagaaggagaggggtctgttgtcgctggctttcccccaagcaggctcttgcacactctagaaaaaacaccttgtaagtctgtgcatttttattgtcttgataaattgtatttttttctaatggggattgggagatggacttcgtttttaaaaatatgtggattttggttaccaagtttagtgttaatatattccatatacatacaaaactacccggtatgtctggcttttcccttctgtcaggtaatagctaaagtcagcatgattgctccctgtaccaccccaaataagtgagtgcctcaccttgtggggcctgagcagctaccttgagaccatgtgaggtggcacctttccggggtggactcgtgcggccttgaggacaggcacagggcaccctatcccaagccgtccaggcaggaggaaggcagccaaggcaactgggttctgggagccctgggtggggcagctgtggggaggaactgggttcggggagccctgggcggggcggctgttggggggaactgggttcggggtgccctgggcagggggctactggggggcggctgtgaggaggagttgggttcagggagccctgggcggggtggctgtcagggggaactgggttccgggagccctgggccggggcagggggcggctgtaggaaggaactggtttcggggagccctgggcggggcggctgtggggaggaaggtgacgtgcaggggaccagaggctctgcactgctcctaggacagctcatctgtaatcagaaaaaaaataaacaaaatacagaacgctgactcctccgtgagacagatcggggaccttagcactttaatccctcccttctgagcgctcggtgtgcacttttagactatagctgtttcattgacgtgtcactctccatccagtgtccttgatgtggcttttagagacttagcagaaaattcgacacaagcaggaacttgattttttaagaaaaaatattacattttgaggacattttgacaagtaggggaagagagggcttctgttgttttgttttgttttgttttgttaactaaacctgaagtattaattccacaaagacactgtccctcaggaccactcaggtacagctctgccagggacagagtcctgctagtgggaggtctcaggtggggcggtgtgttctgtgccatgaggcagcgacaggtccagatggatgtcgtcaccaccttcctcagctctcatcacctggtcgtacgccaggcccacctcttcccagcaagggacgccaaagaactgcagtttttattctgagtcttaatttaacttttcatcatcttttcctattttggagaattttttgtaattaaaagcaattattttaaaatgtgcaagccagtatctcacaaggcatggatttctgtggaatttatttttattcaaataaccatatttatctccaggctgtggaatcgccactttctttgtgaagacagtgtctctccttgtaatctcacacaggtacactgaggaggggacggctccgtcttcacattgtgcacagatctgaggatgggattagcgaagctgtggagactgcacatccggacctgcccatgtctcaaaacaaacacatgtacagtggctctttttccttctcaaacactttaccccagaagcaggtggtctgccccaggcataaagaaggaaaattggccatctttcccacctctaaattctgtaaaattatagacttgctcaaaagattcctttttatcatccccacgctgtgtaagtggaaagggcattgtgttccgtgtgtgtccagtttacagcgtctctgccccctagcgtgttttgtgacaatctccctgggtgaggagtgggtgcacccagccccgaggccagtggttgctcggggccttccgtgtgagttctagtgttcacttgatgccggggaatagaattagagaaaactctgacctgccgggttccagggactggtggaggtggatggcaggtccgactcgaccatgacttagttgtaagggtgtgtcggctttttcagtctcatgtgaaaatcctcctgtctctggcagcactgtctgcactttcttgtttactgtttgaagggacgagtaccaagccacaagaacacttcttttggccacagcataagctgatggtatgtaaggaaccgatgggccattaaacatgaactgaacggttaaaagcacagtctatggaacgctaatggagtcagcccctaaagctgtttgctttttcaggctttggattacatgcttttaatttgattttagaatctggacactttctatgaatgtaattcggctgagaaacatgttgctgagatgcaatcctcagtgttctctgtatgtaaatctgtgtatacaccacacgttacaactgcatgagcttcctctcgcacaagaccagctggaactgagcatgagacgctgtcaaatacagacaaaggatttgagatgttctcaataaaaagaaaatgtttcacta

SEQ ID NO: 10 - protein, 701 aa, Artificial
  Synthetic
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 11 - DNA, 1704 bp, Artificial
  Synthetic
agcgtgggcggggctgtgccggggcagcccggtaaaaaagagcgtggcgggccgcggtctctgagagccatcgggaagcgaccctgccagcgagccaacgcagacccagagagcttcggcggagagaaccgggaacacgctcggaaccatggcccagacacccgcattcaacaaacccaaagtagagttacacgtccacctggatggagccatcaagccagaaaccatcttatactttggcaagaagagaggcatcgccctcccggcagatacagtggaggagctgcgcaacattatcggcatggacaagcccctctcgctcccaggcttcctggccaagtttgactactacatgcctgtgattgcgggctgcagagaggccatcaagaggatcgcctacgagtttgtggagatgaaggcaaaggagggcgtggtctatgtggaagtgcgctatagcccacacctgctggccaattccaaggtggacccaatgccctggaaccagactgaaggggacgtcacccctgatgacgttgtggatcttgtgaaccagggcctgcaggagggagagcaagcatttggcatcaaggtccggtccattctgtgctgcatgcgccaccagcccagctggtcccttgaggtgttggagctgtgtaagaagtacaatcagaagaccgtggtggctatggacttggctggggatgagaccattgaaggaagtagcctcttcccaggccacgtggaagcctatgagggcgcagtaaagaatggcattcatcggaccgtccacgctggcgaggtgggctctcctgaggttgtgcgtgaggctgtggacatcctcaagacagagagggtgggacatggttatcacaccatcgaggatgaagctctctacaacagactactgaaagaaaacatgcactttgaggtctgcccctggtccagctacctcacaggcgcctgggatcccaaaacgacgcatgcggttgttcgcttcaagaatgataaggccaactactcactcaacacagacgaccccctcatcttcaagtccaccctagacactgactaccagatgaccaagaaagacatgggcttcactgaggaggagttcaagcgactgaacatcaacgcagcgaagtcaagcttcctcccagaggaagagaagaaggaacttctggaacggctctacagagaataccaatagccaccacagactgacgcagggcgggtcccctgaagatggcaaggccacttctctgagcctcatcctgtggataaagtctttacaactctgacatattgaccttcattccttccagaccttggagaggccaggtctgtcctctgattggatatcctggctaggtcccaggggacttgacaatcatgcacatgaattgaaaaccttccttctaaagctaaaattatggtgttcaataaagcagctggtgactggtatcttgcagcacatggtgaatatggtctcggggctgctggctaggatgctaagaaaggaggagccctgggccctacgctgagtgtcaggctggggagccagggtctctttcctgcagaagcgattctttcccagaggggctgttggagcagatgctcctgaactctccgcccctttaaccagtcctttggatttatttttattatttttaaatatttaattatgtttatgtatatgggtgtttt

SEQ ID NO: 12 - DNA, 6244 bp, Artificial
  Synthetic
ctctgccgcgggctctgtagctgagtggtggctgggtatggaggcgaaggcggcacccaagccagctgcaagcggcgcgtgctcggtgtcggcagaggagaccgaaaagtggatggaggaggcgatgcacatggccaaagaagccctcgaaaatactgaagttcctgttggctgtcttatggtctacaacaatgaagttgtagggaaggggagaaatgaagttaaccaaaccaaaaatgctactcgacatgcagaaatggtggccatcgatcaggtcctcgattggtgtcgtcaaagtggcaagagtccctctgaagtatttgaacacactgtgttgtatgtcactgtggagccgtgcattatgtgtgcagctgctctccgcctgatgaaaatcccgctggttgtatatggctgtcagaatgaacgatttggtggttgtggctctgttctaaatattgcctctgctgacctaccaaacactgggagaccatttcagtgtatccctggatatcgggctgaggaagcagtggaaatgttaaagaccttctacaaacaagaaaatccaaatgcaccaaaatcgaaagttcggaaaaaggaatgtcagaaatcttgaacatgttctgatgaaagaaccaagtgacccaaagtgacctggacaagattcatagactgaaagctgttgacatcgttgaatcatatgtttatatattgtttttaatctgcaggaaaatggtgtctctcatcatttgctctgttaagggaacaaattagcactttttagaagtctgacaattgtaaacagttattagcttttccagaagctgattcccattttaagatgggggaaaattaaggtttgaggttttagaaattagcaagtagtgcatacccttctagccacaagtgcccagtccaggcaagtgctgacttcttagagaatgtgtggccagacccagggacctggagtgtgtttggactgcagtttgccaccctgagaacaccttctccaggactggcatttcagaatcagattcttcattttttgcagctacgatgttcttccagggcactgggggctgtgacttctctctaaattgtatataagttgtgtatatagagaccataattatatggtccttagaaaagactttgcttttataaagcatttagaaaaaatgcatacttttaaaacaagtgcttgagttgtcacttaaaaattatagcatattgctataataaaaccttatttatgtcttatttgaagatgaatagtcttaaaagataaagacataaatgggacaattgttattgagcaaaaaaccaaattatcccaccctcatggagcttatattctagcaaggggagatggatatgatagattacacagtttattggaggacaataagagttatggcaaaaagcaaaaggaacacagggtaaaggggataggtgccatttggtggtgagaatgctgactgaaaaatagaatgatcaatttaatctgaaacaaatggttatttcttttataatccatataataaatttaaaatctaaaatgtaaaattttgaacacaacactggaaagggtatccacagcaggaagtccccagttcacctccatgactacagggcagctttgcacagccctctgggcgcactgtgtgcctctgcccagaagggggcctcgccgttccaccagaagctcagctccaggccctggaggggctgctgctcctcagttgcatttcttcagtagattcatttccttgatgcaaagcatctgtatttgttggttctgtcatttgagcgatgtctctgacttgtttgttttgaattacattacaggctggaatgtaattgtggtgaaagtatttttatattgctgagagtagcagctaatcacagttacatgcttcagaggacttataattgcttggttttgtgtgtgtgtgtgtgtgtgtgtgtgtgtgtgtgtgtgtttaactgcatttgaaaagttttatggagaatatgcatgattttaaatctgtgataatgttacatgcaccttcaatttcatccactttaaaaattatcttctcattgaattttagtgcttctactagtttgttcctttttgcagttggtcgtaattcatttctggcttcttatgctttcctgcaagcagatttcattgcatttattgtgttcatatcattttcttggggattatttgtaggacaaccaacctggagttttgcctctctagagtaccacccagtaagtctggctgagcatcttatgtccagtaggttcttggtaaacatttgctaaatgaaattactgattgaaatttggggaaaagtgaataagaagactatctaggacaaaaagccaaagccgaaaatagtatatgagcattctagcccagagactgtcgctactaaaagaatgaaggaaataataaagtgatagacagggaaggatagaaaagacttaacaatatacatatgttccgtctttgctgttttggagaatgatggataagtagtgtttcctgattctgaagcatagctgaacaatttaattgtggtttaccatctttttggttccctcttcagtaattaacctatcgaaaatctgtcctaaatgtttggactggggcacagttccctccatcgctttgggagaaaatcattaatatggcatactgcagattggagggcaggaccactgagggtgtcatagacattagctctatggaattctgctagcaatttccaagtgacagtgaggaattatggatatatgttgaggtcattcagcttcctgagtaccacattccccagctacttagacacgggttaaaatattaagatgtcctagttcaacagcttgaattccattgattgatactgatagtgcctgtccaagacaccagctgaaagacttgttttgtgtacaaaatagttctgaaagtggtgagatacaaaaaggttttagaatcactgccctgttgagagaaattagggggaaatgattacatttagaagctgctagagttatccagtgtttgctggtctttgcaacaaactgtggagaatgggtggtatgtaatgctttggtaggcttcaatcactgataaaagatcatgttaaaatatctttgtgctttcttgttacttggcacaaccatctcttcctgtgttgtatttggagtatcatggagagaaaatagatggccaagagcttcagtgtaggcaagaactcttaatttttctttaaactttttactgggaaaagtatatatatataaaatacacacacacacacacacacacacacacacacacacacacacacaaacacaacacaccatggccctttaccccgaaatgcttcagtatagttattgacttaagtaaatttaacattgatatacttgaatctatcatttgtattacagttttgtcagctgacccaataatgtcctgtaaagaagttctcccactaccctataatcccaggtccagtctagggtccagcattacatttacttgtcttgaatccagctttttcttttttttttttttttttgagataggtctcactctgtcgtccagtggcatgatcacagctcactgcagcctcaacctggctcaagcaatccttcctcctcagcctcctgagtagctgggaccacagactcatgtcaccacacctaatttttttttttttttttttttgtagagacaaggtctcactatgttgcccaggctggtcttgaactcctaggctgaagcaatcctccttccttggcctcccaaagcactgggattatagacgtgagccactgcaccggtctgcctttagcttcttttagtctagaacattttcactggctttctttgtcttttatgacattgacatttttaaataatacagtcattttgcctcctttctgttttcttcttctttttttaaataatagaatggtccttgttttaaatttatttgatattttcttgtgattagattcaggtgctggttgatgttaagttcctcacaggatatcacatctggaggcacacaaaggccgtcacaccaaggtgatgtcaattttggtcatctggtcaaggtgttgtcctattccttcactatatagttaccttttttctctgttgcaatgaataagcagtctgtgggaagaggagctgttacattttaaacagaaaatgtatttgacactgatggaaaggagaggaggaaaattaatgacataaatttcaaagcaactattaaattatttgattgcattcttcctcttttactgtctgccaaaattgataaaaaaaatttttctaataagaatgttttaaatagtgatatcttaataagcatcaaaattaagcctgagaaataaattctttccttcctaatttcctcctcagcaaaagtaataattatataaatttcattatgcctgataagatagggttttggaaaatagacctaagatgtttctgatactgcagatgacctatggtgatccaatgggataaacactctaggtaggttgtcatttggtcataaaatatgagttatcttgggtttccatagagacatctagacttaaaatgttgtaagcactgctactttcaaaatgtcagtaaaaatagcaaaagccaaagctcttgaaaaaattacttaaatcttttttaaaagtagtatagcgccttgttaaaaatctgtggtgatgccaaagcttgtctttcccagtggtcctacgtgaactggccttatagccccagggaaaccagacaccaggaattggtttctctgccttttggcaaaggaataagactacattgacttcatctatgaagacaactgccaactatttcctttgtaaattgctaattttgtgtagtgaggaaaggagcgatgggcgacgtgatttttatggattagactggtgagttctgctgaaagtttgacatctttaggatcttacattttcttcaagttgagctaatgaaaacaggctcgtgactatttatcacctgatttctaagtggatattgggttgaacaccacatatccatgactattaaggaggcttcatggtgtagtttgacaaaggctctctccttgaccaaacttcagtcaggccctaagtcctctttttaaccaggcctccaccttggcccccattcttgatgggcctatacagcccagctttagcaagaatcctgctaagctagtttagagagaatcccacatccccaatatctatgaaatttctcatcccctacttttgatgtgtaagtccttggcctcccttcaacgagaagcctgttaagttcattttgcaagaactctactcttgatatctcctcttagtaatttcctaatcactgaccccctcactctgcccattagttataaacccccacatgttctggttgtattcagagctgagcctgatctcttcctcttgttgggatagttttaaaacctgcgatagttttaaaacctatcactgtagtcctgaattaagtcttccttaccttaacaagtgtcaaaataaatttttctttaacatgttgaagcatgaacttgagaatctagagcaggagtccacaaagtatggcccatgggccatatccagcccgctgccggtttcggtaccactcatgacttaaaaatgggtcttacaattctgagtgattgaaaaaaaatcaaaagaaggataatatttagtgacccatgaaccttatatggcaatcaaatttcagtgtccataaataaagttacattggatgacagccatgcccatttgtttctgtgttgtctgtggctgctcgtgtgctacaatggcagagttgagcagtggtgacaaaccatgcgactcacaaaggcctaaaatatttagcgtctggcccttcgagaaaatgttagctgcccctggtctagagtaggtaaaaggctgagattggaagctgcttgttcaaattctgtgattggaaccgaatgatgtggctcattgtacagctcatggtgaattgcttcagtaccatggttttgttttttccttttgaaaagttggtctataaatgtaaaggaaaaatctaagataccaaaatatgttttctggcttagaatgttttatttccttgtatacattttaagagagtggcaaggagaaaagataatgtatcattttatttgggtttagaataaataatacattttatttatgatca

SEQ ID NO: 13 - protein, 191 aa, Artificial
  Synthetic
MEAKAAPKPAASGACSVSAEETEKWMEEAMHMAKEALENTEVPVGCLMVYNNEVVGKGRNEVNQTKNATRHAEMVAIDQVLDWCRQSGKSPSEVFEHTVLYVTVEPCIMCAAALRLMKIPLVVYGCQNERFGGCGSVLNIASADLPNTGRPFQCIPGYRAEEAVEMLKTFYKQENPNAPKSKVRKKECQKS

SEQ ID NO: 14 - protein, 352 aa, Artificial
  Synthetic
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 15 - DNA, 2803 bp, Artificial
  Synthetic
gtcagactaagacagagaaccatcattaattgaagtgagatttttctggcctgagacttgcagggaggcaagaagacactctggacaccactatggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttgatagcaacttccaggaatgtcacacacgatgaaatatctctgctgaagacagtggataaaaaacagtccttcaagtcttctctgtttttattcttcaactctcactttcttagagtttacagaaaaaatatttatatacgactctttaaaaagatctatgtcttgaaaatagagaaggaacacaggtctggccagggacgtgctgcaattggtgcagttttgaatgcaacattgtcccctactgggaataacagaactgcaggacctgggagcatcctaaagtgtcaacgtttttctatgacttttaggtaggatgagagcagaaggtagatcctaaaaagcatggtgagaggatcaaatgtttttatatcaacatcctttattatttgattcatttgagttaacagtggtgttagtgatagatttttctattcttttcccttgacgtttactttcaagtaacacaaactcttccatcaggccatgatctataggacctcctaatgagagtatctgggtgattgtgaccccaaaccatctctccaaagcattaatatccaatcatgcgctgtatgttttaatcagcagaagcatgtttttatgtttgtacaaaagaagattgttatgggtggggatggaggtatagaccatgcatggtcaccttcaagctactttaataaaggatcttaaaatgggcaggaggactgtgaacaagacaccctaataatgggttgatgtctgaagtagcaaatcttctggaaacgcaaactcttttaaggaagtccctaatttagaaacacccacaaacttcacatatcataattagcaaacaattggaaggaagttgcttgaatgttggggagaggaaaatctattggctctcgtgggtctcttcatctcagaaatgccaatcaggtcaaggtttgctacattttgtatgtgtgtgatgcttctcccaaaggtatattaactatataagagagttgtgacaaaacagaatgataaagctgcgaaccgtggcacacgctcatagttctagctgcttgggaggttgaggagggaggatggcttgaacacaggtgttcaaggccagcctgggcaacataacaagatcctgtctctcaaaaaaaaaaaaaaaaaaaagaaagagagagggccgggcgtggtggctcacgcctgtaatcccagcactttgggaggccgagccgggcggatcacctgtggtcaggagtttgagaccagcctggccaacatggcaaaaccccgtctgtactcaaaatgcaaaaattagccaggcgtggtagcaggcacctgtaatcccagctacttgggaggctgaggcaggagaatcgcttgaacccaggaggtggaggttgcagtaagctgagatcgtgccgttgcactccagcctgggcgacaagagcaagactctgtctcagaaaaaaaaaaaaaaaagagagagagagagaaagagaacaatatttgggagagaaggatggggaagcattgcaaggaaattgtgctttatccaacaaaatgtaaggagccaataagggatccctatttgtctcttttggtgtctatttgtccctaacaactgtctttgacagtgagaaaaatattcagaataaccatatccctgtgccgttattacctagcaacccttgcaatgaagatgagcagatccacaggaaaacttgaatgcacaactgtcttattttaatcttattgtacataagtttgtaaaagagttaaaaattgttacttcatgtattcatttatattttatattattttgcgtctaatgattttttattaacatgatttccttttctgatatattgaaatggagtctcaaagcttcataaatttataactttagaaatgattctaataacaacgtatgtaattgtaacattgcagtaatggtgctacgaagccatttctcttgatttttagtaaacttttatgacagcaaatttgcttctggctcactttcaatcagttaaataaatgataaataattttggaagctgtgaagataaaataccaaataaaataatataaaagtgatttatatgaagttaaaataaaaaatcagtatgatggaataaa

SEQ ID NO: 16 - protein, 198 aa, Artificial [sequence omitted]

SEQ ID NO: 17 - DNA, 11382 bp, Artificial
  Synthetic
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccatggacagcctcttgatgaaccggagggagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcatctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacggaagaactttcaaagcctgggaagggctgcatgaaaattcagttcgtctctccagacagcttcggcgcatccttttgcccctgtatgaggttgatgacttacgagacgcatttcgtacttgtacaggcagtggagagggcagaggaagtctgctaacatgcggtgacgtcgaggagaatcctggcccaaccatgaaaaagcctgaactcaccgctacctctgtcgagaagtttctgatcgaaaagttcgacagcgtctccgacctgatgcagctctccgagggcgaagaatctcgggctttcagcttcgatgtgggagggcgtggatatgtcctgcgggtgaatagctgcgccgatggtttctacaaagatcgctatgtttatcggcactttgcatccgccgctctccctattcccgaagtgcttgacattggggagttcagcgagagcctgacctattgcatctcccgccgtgcacagggtgtcaccttgcaagacctgcctgaaaccgaactgcccgctgttctccagcccgtcgccgaggccatggatgccatcgctgccgccgatcttagccagaccagcgggttcggcccattcggacctcaaggaatcggtcaatacactacatggcgcgatttcatctgcgctattgctgatccccatgtgtatcactggcaaactgtgatggacgacaccgtcagtgcctccgtcgcccaggctctcgatgagctgatgctttgggccgaggactgccccgaagtccggcacctcgtgcacgccgatttcggctccaacaatgtcctgaccgacaatggccgcataacagccgtcattgactggagcgaggccatgttcggggattcccaatacgaggtcgccaacatcttcttctggaggccctggttggcttgtatggagcagcagacccgctacttcgagcggaggcatcccgagcttgcaggatctcctcggctccgggcttatatgctccgcattggtcttgaccaactctatcagagcttggttgacggcaatttcgatgatgcagcttgggctcagggtcgctgcgacgcaatcgtccggtccggagccgggactgtcgggcgtacacaaatcgcccgcagaagcgctgccgtctggaccgatggctgtgtggaagtgctcgccgatagtggaaacagacgccccagcactcgtcctagggcaaaggatctgcagtaatgagaattcgatatcaagcttatcggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 18 - protein, 195 aa, Artificial
  Synthetic
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRT

SEQ ID NO: 19 - DNA, 5477 bp, Artificial
  Synthetic
agaaaaatcctattggcattgaggaggtagggagccagcccctgggcgcggcctgcagggtaccggcaaccgcccgggtaagcgggggcaggacaaggccggagcctgtgtccgcccggcagccgcccgcagctgcagagagtcccgctgcgtctccgccgcgtgcgccctcctcgaccagcagacccgcgctgcgctccgccgctgacatgtgtgccgctcagatgccgcccctggcgcacatcttccgagggacgttcgtccactccacctggacctgccccatggaggtgctgcgggatcacctcctcggcgtgagcgacagcggcaaaatagtgtttttagaagaagcatctcaacaggaaaaactggccaaagaatggtgcttcaagccgtgtgaaataagagaactgagccaccatgagttcttcatgcctgggctggttgatacacacatccatgcctctcagtattcctttgctggaagtagcatagacctgccactcttggagtggctgaccaagtacacatttcctgcagaacacagattccagaacatcgactttgcagaagaagtatataccagagttgtcaggagaacactaaagaatggaacaaccacagcttgttactttgcaacaattcacactgactcatctctgctccttgccgacattacagataaatttggacagcgggcatttgtgggcaaagtttgcatggatttgaatgacacttttccagaatacaaggagaccactgaggaatcgatcaaggaaactgagagatttgtgtcagaaatgctccaaaagaactattctagagtgaagcccatagtgacaccacgtttttccctctcctgctctgagactttgatgggtgaactgggcaacattgctaaaacccgtgatttgcacattcagagccatataagtgaaaatcgtgatgaagttgaagctgtgaaaaacttataccccagttataaaaactacacatctgtgtatgataaaaacaatcttttgacaaataagacagtgatggcacacggctgctacctctctgcagaagaactgaacgtattccatgaacgaggagcatccatcgcacactgtcccaattctaatttatcgctcagcagtggatttctaaatgtgctagaagtcctgaaacatgaagtcaagatagggctgggtacagacgtggctggtggctattcatattccatgcttgatgcaatcagaagagcagtgatggtttccaatatccttttaattaataaggtaaatgagaaaagcctcaccctcaaagaagtcttcagactagctactcttggaggaagccaagccctggggctggatggtgagattggaaactttgaagtgggcaaggaatttgatgccatcctgatcaaccccaaagcatccgactctcccattgacctgttttatggggacttttttggtgatatttctgaggctgttatccagaagttcctctatctaggagatgatcgaaatattgaagaggtttatgtgggcggaaagcaggtggttccgttttccagctcagtgtaagaccctcgggcgtctacaaagttctcctgggattagcgtggttctgcatctcccttgtgcccaggtggagttagaaagtcaaaaaatagtaccttgttcttgggatgactatccctttctgtgtctagttacagtattcacttgacaaatagttcgaaggaagttgcactaattctcaactctggttgagagggttcataaatttcatgaaaatatctccctttggagctgctcagacttactttaagctcaaacagaagggaatgctattactggtggtgttcctacggtaagacttaagcaaagcctttttcatatttgaaaatgtggaaagaaaagatgttcctaaaaggttagatattttgagctaataattgcaaaaattagaagactgaaaatggacccatgagagtatatttttatgagggagcaaaagttagactgagaacaaacgttagaaaatcacttcagattgtgtttgaaaattatatactgagcatactaatttaaaaagagaacttgttgaaatttaaaacgtgtttctaggttgaccttgtgttttagaaatttgcacttaatggaatttgcatttcagagatgtgttagtgttgtgctttgccttctttggcgatgaatgtcagaaattgaatgccacatgctttcataatatagttttgtgcttcaaagtgtttgacagaagttgggtattaaagatttaaagtctcttaggaatattattcatgtaactccatggcataaatagttgtatttttgtgtactttaaaatcaacttataactgtgagatgttattgcttccattttattagaagagaaacaaattccatgctttatggaatttatgtagactggagtcttcgtgaactggggcaaatgctggcatccaggagccgccaatactaacaggacaggttccattgccatggcctattccacccaaacaatatgttgtagtttctggaaattccatactcagatatcagtctgctagaactttaaaatgaaggacaaatcctgttaaagaaatattgttaaaaatctttaaaccctgtgtattgaaagcactctattttctaattttatccagttttctgtttaactccttataatgtttaggatattaaaattttaggataatgaagagtacataatgtcctacttaatatttatgttaataggacttaattcttactagacatctaggaacattacaaagcaaagactatttttatgcttccataacctagaattaaaaccaaattatgaccttatgataaatctttaagtattggtgtgaatgttatttaaattctatatttttcttatttaattacaaatactataaatgagcaaggaaaaggaatagactttcttaatatattataacactcattcctagagcttaggggtgactctttaatattaccttatagtagaaactttatgtaatatagctaactccgtatttacagaacaaaaaaacacagttccccctcctgtagtataaattttattttcacatacttagctaatttagcagtaattggcccagttttttccctaatagaaatacttttagatttgattatgtatacatgacacctaaagagggaacaaaagttagttttatttttttaataaacaacagagtttgttttgtgagataagtatcttagtaaacccaatttccagtcttagtctgtatttccaatatttctaattcctgagccacgtcaaagatgccttgccaaatttctccccatttctctacggggctagcaaaaatcttcagctttatcactcaacccctgccaaaggaacttgattacatggtgtctaaccaaatgagcaggcttaggaatttagatgagatgtgtaagattcacttacaggcagtagctgcttctagcatttgcaagatcctacacttttaccttctttaagggtgtacattttgatgttgaacatcagttttcatgtagacttaggactcatgtgcagtaaatataaataagtgtagcatcagaagcagtaggaatggccgtatacaaccatcctgttaaacatttaaatttagctctgatagtgtgttaagacctgaatatctttcctagtaaaaataggatgtgttgaaatatttatatgtactttgatctctccacatcacttataacttatgtgttttatttctccaagtgcggtgttcctgaatgttatgtatgcttttttttctgtaccacaggcattatctatacctggggccagattttctgcactttgaaatgttgcctttgcctaatgtaggttgactttctgaattgtggagaggcacttttccaagccaatcttatttgtcactttttgttttaatatcttgctctctgacaggaaagaaacaattcacttaccagcctcctcaccccatcctccaccatttccttaatgttccatggtattttcaacggaatacactttgaaaggtaaaaacaattcaaaagtatcgattatcataaattcacaaaatatttttgcaaccagaacacaaaagcaggctagtcagctaaggtaaatttcattttcaaacgagagggaaacatgggaagtaaaagattaggatgtgaaaggttgtcctaaacagaccaaggagactgttccctaatttattctcttggctggttctctcattgaattatcagaccccaagaggagatattggaacaggctcccttcatgccaagggtctttctaagttaatactgtgagcattgagcccccattaaaactcttttttacttcagaaagaattttacaggttaaagggaaagaaatggtgggaaactctccccgtaatgcttagccaactttaaagtgtacccttcaatatccccattggcaactgcagctgagatcttagagaggaaatataaccggtgtgagatctagcaatgcattttgaatcttcactccctaccaggctcttcctatttttaatctcttcacctcagaactagacatatggagagctttaaaggcaagctggaaggcacattgtatcaattctaccttgtgctatacgtaggagagatccaaaatttggatgcttctggagactcttagacatcttttcattgttgtccatttttaaagttgatgattgctggaaacattcacacgcttaaaagcaatggtgtgagttattaatgggtaaactaagaagtgttataggcaatgacttgaaatggtttttaaattgtatggattgttaagaattgttgaaaaaaaattttttttttttggacagcttcaaggagatgttagcaatttcagatatactagccagtttaggtatgactttggaagtgcagaaacagaaggatactgttagaaaatcctaacattggtctccgtgcatgtgttcacacctggtctcactgcctttccttcccacagacctgagtgtgaaagactgagagttgaggagttactttgtggatcttgtccaaatttagtgaaatgtggaagtcaaccagaccaatgatggaattaaatgtaaattccaagagggctttcacagtccacagggttcaaatgacttgggtaacagaagttattcttagcttacctgttatgtgacagtgatttacctgtccatttccaacccaaaagcctgtcagaaagcattctttagagaaaaccactttacatttgttgttaaactcctgatcgctactcttaagaatatacatgtatgtattcataggaacattttttctcaatatttgtatgattcgcttactgttattgtgctgagtgagctcctgtgtgcttcagacaaaaataaatgagactttgtgtttacgttaaaaaaaaaaaaaaaaaaaaaa

SEQ ID NO: 20 - protein, 454 aa, Artificial
  Synthetic
MCAAQMPPLAHIFRGTFVHSTWTCPMEVLRDHLLGVSDSGKIVFLEEASQQEKLAKEWCFKPCEIRELSHHEFFMPGLVDTHIHASQYSFAGSSIDLPLLEWLTKYTFPAEHRFQNIDFAEEVYTRVVRRTLKNGTTTACYFATIHTDSSLLLADITDKFGQRAFVGKVCMDLNDTFPEYKETTEESIKETERFVSEMLQKNYSRVKPIVTPRFSLSCSETLMGELGNIAKTRDLHIQSHISENRDEVEAVKNLYPSYKNYTSVYDKNNLLTNKTVMAHGCYLSAEELNVFHERGASIAHCPNSNLSLSSGFLNVLEVLKHEVKIGLGTDVAGGYSYSMLDAIRRAVMVSNILLINKVNEKSLTLKEVFRLATLGGSQALGLDGEIGNFEVGKEFDAILINPKASDSPIDLFYGDFFGDISEAVIQKFLYLGDDRNIEEVYVGGKQVVPFSSSV

SEQ ID NO: 21 - protein, 84 aa, Artificial
  Synthetic
MTNLSDIIEKETGKQLVIQESILMLPEEVEEVIGNKPESDILVHTAYDESTDENVMLLTSDAPEYKPWALVIQDSNGENKIKML

SEQ ID NO: 22 - protein, 7 aa, Artificial
  Synthetic
PKKKRKV

SEQ ID NO: 23 - protein, 16 aa, Artificial
  Synthetic
KRPAATKKAGQAKKKK

SEQ ID NO: 24 - protein, 4 aa, Artificial
  Synthetic
  misc_feature (2)..(4): Xaa can be any naturally occurring amino acid
KXXX

SEQ ID NO: 25 - protein, 20 aa, Artificial
  Synthetic
AVKRPAATKKAGQAKKKKLD

SEQ ID NO: 26 - protein, 25 aa, Artificial
  Synthetic
MSRRRKANPTKLSENAKKLAKEVEN

SEQ ID NO: 27 - protein, 9 aa, Artificial
  Synthetic
PAAKRVKLD

SEQ ID NO: 28 - protein, 9 aa, Artificial
  Synthetic
KLKIKRPVK

SEQ ID NO: 29 - DNA, 576 bp, Artificial
  Synthetic
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 30 - DNA, 585 bp, Artificial
  Synthetic
atggacagcctcttgatgaaccggagggagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcatctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacggaagaactttcaaagcctgggaagggctgcatgaaaattcagttcgtctctccagacagcttcggcgcatccttttgcccctgtatgaggttgatgacttacgagacgcatttcgtact

SEQ ID NO: 31 - DNA, 6695 bp, Artificial
  Synthetic
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgagagccgccaccatggacagcctcttgatgaaccggagggagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcatctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacggaagaactttcaaagcctgggaagggctgcatgaaaattcagttcgtctctccagacagcttcggcgcatccttttgcccctgtatgaggttgatgacttacgagacgcatttcgtactagcggcagcgagactcccgggacctcagagtccgccacacccgaaagtaacaccatcaacattgctaagaacgacttctcagacatagagctcgcggctattccgttcaacaccctggctgaccactacggcgagagactcgctagggagcagctggcgttggagcatgaatcctacgagatgggcgaggctaggttccgcaagatgttcgagcgacaattgaaggcaggggaggtggcggacaacgctgccgccaagcccctgatcacaaccttgctgcccaaaatgatcgcgcggatcaacgattggtttgaggaggttaaggcaaaacggggcaaacgcccgaccgcatttcaattcctccaagaaatcaagcctgaggctgttgcctacatcactatcaagacgacactggcgtgtctcacaagcgccgacaacaccaccgtgcaagccgtcgccagcgccatcgggcgggcaattgaggatgaggcacggtttggtaggatccgagacctggaagcgaagcacttcaagaagaacgtggaagagcagttgaacaaacgcgtcggccacgtgtataaaaaggctttcatgcaggtggtggaggccgatatgctcagtaaggggctgcttgggggggaggcgtggtcatcctggcacaaggaggatagcattcacgtgggggtccgatgtatcgagatgctgatagagagcaccggaatggtctccctccatcgccagaacgctggggtcgtagggcaggactccgagactattgagctggcccccgagtatgccgaagcaatcgctacacgcgcaggtgcactggctgggataagccctatgtttcagccctgcgtagtgcctccaaagccatggaccggcatcacagggggtggctattgggccaacggtaggcggcctctggccctggtacgcacgcacagcaagaaggcgctcatgcgctatgaagacgtttacatgcccgaggtttacaaggcgatcaatatcgcgcagaacaccgcctggaaaatcaataagaaggtgttggcggtcgcaaacgtgattaccaagtggaagcattgcccagtcgaggacatacccgccatagaacgcgaagagctgccgatgaagccggaagacattgatatgaaccccgaggccctcaccgcgtggaaaagagccgcagccgccgtatacaggaaggataaagcgcgcaagtcccgacgcataagcctcgagtttatgctggaacaggccaacaagttcgccaaccacaaagctatctggttcccctacaacatggactggagagggagggtctacgccgtcagcatgttcaatccccagggcaacgacatgacgaagggccttctgacattggcaaaggggaagcctatcggaaaggaggggtactactggctcaagatccacggcgccaactgcgcgggagtggacaaggttccatttcccgagcgaattaagttcatcgaggaaaaccacgaaaacattatggcgtgcgctaaatcccccctcgagaacacatggtgggccgagcaagactccccgttctgttttttggcattctgctttgagtacgccggtgtgcagcaccatggcctctcatacaactgttccctgcccctggccttcgacggaagttgcagtgggattcaacatttcagcgcaatgttgcgggacgaggtcggtggcagggccgttaacctgctcccttccgaaacggtgcaggacatctacggaatcgtggcaaaaaaggtaaacgagatcctgcaagcggatgccatcaacgggacggacaatgaggtcgttacggtgacagacgaaaatactggggaaataagcgaaaaggtcaagctggggaccaaagcactcgcgggtcagtggctcgcctacggggtgacacgctccgtcaccaagagaagcgtgatgaccctcgcgtacggttcaaaagaattcggcttccgccagcaagtgctggaggacaccatccagccggcgattgactccgggaagggtctcatgtttacccagccgaaccaggccgcagggtacatggccaaactgatctgggaaagcgttagcgtcacagtggtcgccgcggttgaggcgatgaattggctgaagagcgcggcaaagctcctcgccgctgaggtgaaggacaaaaagaccggcgaaatcctgcgcaagcgctgcgccgtccactgggtcacgccggatggattccccgtctggcaggagtacaagaagcccatccaaacccggctcaacttgatgttccttggccagtttcgcctgcagcccacgataaacaccaacaaagacagcgagatcgacgcccacaagcaggagagcggcatcgcgcccaacttcgtgcacagtcaggacgggtcccatctgcggaaaactgttgtgtgggctcacgagaagtacggcattgagagcttcgccctgatacacgacagcttcgggaccataccagcggacgcagcgaacctgttcaaagccgtgcgggaaacaatggtcgacacctacgaaagctgcgacgtactggcagacttctatgaccaattcgccgaccagcttcacgagtcacagctcgacaagatgcccgctctgcccgcgaaaggcaacctgaatttgcgcgacatccttgagagcgattttgcgttcgcctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 32 - protein, 1104 aa, Artificial
  Synthetic
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTSGSETPGTSESATPESNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFASGGSPKKKRKV

SEQ ID NO: 33 - DNA, 6695 bp, Artificial; identical to SEQ ID NO: 31
  Synthetic

SEQ ID NO: 34 - protein, 1191 aa, Artificial
  Synthetic
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTSGSETPGTSESATPESNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFASGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 35 - DNA, 502 bp, Artificial
  Synthetic
aatgtccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgac

SEQ ID NO: 36 - protein, 167 aa, Artificial; identical to SEQ ID NO: 7
  Synthetic

SEQ ID NO: 37 - DNA, 687 bp, Artificial
  Synthetic
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaa

SEQ ID NO: 38 - DNA, 2619 bp, Artificial
  Synthetic
caagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtacctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagagcgctggatggttgatacaggtatcgaagtacctgagcaaggggagttcgaccttaacgaaatcatggattctgaatacgtatttgcc

SEQ ID NO: 39 - protein, 873 aa, Artificial
  Synthetic
QDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEERWMVDTGIEVPEQGEFDLNEIMDSEYVFA

SEQ ID NO: 40 - protein, 21 aa, Artificial
  Synthetic
CCCAAGAAGAAGAGGAAAGTC

SEQ ID NO: 41 - protein, 7 aa, Artificial
  Synthetic
PKKKRKV

SEQ ID NO: 42 - DNA, 2649 bp, Artificial
  Synthetic
atgaacaccatcaacattgctaagaacgacttctcagacatagagctcgcggctattccgttcaacaccctggctgaccactacggcgagagactcgctagggagcagctggcgttggagcatgaatcctacgagatgggcgaggctaggttccgcaagatgttcgagcgacaattgaaggcaggggaggtggcggacaacgctgccgccaagcccctgatcacaaccttgctgcccaaaatgatcgcgcggatcaacgattggtttgaggaggttaaggcaaaacggggcaaacgcccgaccgcatttcaattcctccaagaaatcaagcctgaggctgttgcctacatcactatcaagacgacactggcgtgtctcacaagcgccgacaacaccaccgtgcaagccgtcgccagcgccatcgggcgggcaattgaggatgaggcacggtttggtaggatccgagacctggaagcgaagcacttcaagaagaacgtggaagagcagttgaacaaacgcgtcggccacgtgtataaaaaggctttcatgcaggtggtggaggccgatatgctcagtaaggggctgcttgggggggaggcgtggtcatcctggcacaaggaggatagcattcacgtgggggtccgatgtatcgagatgctgatagagagcaccggaatggtctccctccatcgccagaacgctggggtcgtagggcaggactccgagactattgagctggcccccgagtatgccgaagcaatcgctacacgcgcaggtgcactggctgggataagccctatgtttcagccctgcgtagtgcctccaaagccatggaccggcatcacagggggtggctattgggccaacggtaggcggcctctggccctggtacgcacgcacagcaagaaggcgctcatgcgctatgaagacgtttacatgcccgaggtttacaaggcgatcaatatcgcgcagaacaccgcctggaaaatcaataagaaggtgttggcggtcgcaaacgtgattaccaagtggaagcattgcccagtcgaggacatacccgccatagaacgcgaagagctgccgatgaagccggaagacattgatatgaaccccgaggccctcaccgcgtggaaaagagccgcagccgccgtatacaggaaggataaagcgcgcaagtcccgacgcataagcctcgagtttatgctggaacaggccaacaagttcgccaaccacaaagctatctggttcccctacaacatggactggagagggagggtctacgccgtcagcatgttcaatccccagggcaacgacatgacgaagggccttctgacattggcaaaggggaagcctatcggaaaggaggggtactactggctcaagatccacggcgccaactgcgcgggagtggacaaggttccatttcccgagcgaattaagttcatcgaggaaaaccacgaaaacattatggcgtgcgctaaatcccccctcgagaacacatggtgggccgagcaagactccccgttctgttttttggcattctgctttgagtacgccggtgtgcagcaccatggcctctcatacaactgttccctgcccctggccttcgacggaagttgcagtgggattcaacatttcagcgcaatgttgcgggacgaggtcggtggcagggccgttaacctgctcccttccgaaacggtgcaggacatctacggaatcgtggcaaaaaaggtaaacgagatcctgcaagcggatgccatcaacgggacggacaatgaggtcgttacggtgacagacgaaaatactggggaaataagcgaaaaggtcaagctggggaccaaagcactcgcgggtcagtggctcgcctacggggtgacacgctccgtcaccaagagaagcgtgatgaccctcgcgtacggttcaaaagaattcggcttccgccagcaagtgctggaggacaccatccagccggcgattgactccgggaagggtctcatgtttacccagccgaaccaggccgcagggtacatggccaaactgatctgggaaagcgttagcgtcacagtggtcgccgcggttgaggcgatgaattggctgaagagcgcggcaaagctcctcgccgctgaggtgaaggacaaaaagaccggcgaaatcctgcgcaagcgctgcgccgtccactgggtcacgccggatggattccccgtctggcaggagtacaagaagcccatccaaacccggctcaacttgatgttccttggccagtttcgcctgcagcccacgataaacaccaacaaagacagcgagatcgacgcccacaagcaggagagcggcatcgcgcccaacttcgtgcacagtcaggacgggtcccatctgcggaaaactgttgtgtgggctcacgagaagtacggcattgagagcttcgccctgatacacgacagcttcgggaccataccagcggacgcagcgaacctgttcaaagccgtgcgggaaacaatggtcgacacctacgaaagctgcgacgtactggcagacttctatgaccaattcgccgaccagcttcacgagtcacagctcgacaagatgcccgctctgcccgcgaaaggcaacctgaatttgcgcgacatccttgagagcgattttgcgttcgcc

SEQ ID NO: 43 - protein, 883 aa, Artificial
  Synthetic
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 44 - DNA, 249 bp, Artificial
  Synthetic
actaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctc

SEQ ID NO: 45 - protein, 83 aa, Artificial
  Synthetic
TNLSDIIEKETGKQLVIQESILMLPEEVEEVIGNKPESDILVHTAYDESTDENVMLLTSDAPEYKPWALVIQDSNGENKIKML

SEQ ID NO: 46 - DNA, 6797 bp, Artificial
  Synthetic
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgagagccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtaacaccatcaacattgctaagaacgacttctcagacatagagctcgcggctattccgttcaacaccctggctgaccactacggcgagagactcgctagggagcagctggcgttggagcatgaatcctacgagatgggcgaggctaggttccgcaagatgttcgagcgacaattgaaggcaggggaggtggcggacaacgctgccgccaagcccctgatcacaaccttgctgcccaaaatgatcgcgcggatcaacgattggtttgaggaggttaaggcaaaacggggcaaacgcccgaccgcatttcaattcctccaagaaatcaagcctgaggctgttgcctacatcactatcaagacgacactggcgtgtctcacaagcgccgacaacaccaccgtgcaagccgtcgccagcgccatcgggcgggcaattgaggatgaggcacggtttggtaggatccgagacctggaagcgaagcacttcaagaagaacgtggaagagcagttgaacaaacgcgtcggccacgtgtataaaaaggctttcatgcaggtggtggaggccgatatgctcagtaaggggctgcttgggggggaggcgtggtcatcctggcacaaggaggatagcattcacgtgggggtccgatgtatcgagatgctgatagagagcaccggaatggtctccctccatcgccagaacgctggggtcgtagggcaggactccgagactattgagctggcccccgagtatgccgaagcaatcgctacacgcgcaggtgcactggctgggataagccctatgtttcagccctgcgtagtgcctccaaagccatggaccggcatcacagggggtggctattgggccaacggtaggcggcctctggccctggtacgcacgcacagcaagaaggcgctcatgcgctatgaagacgtttacatgcccgaggtttacaaggcgatcaatatcgcgcagaacaccgcctggaaaatcaataagaaggtgttggcggtcgcaaacgtgattaccaagtggaagcattgcccagtcgaggacatacccgccatagaacgcgaagagctgccgatgaagccggaagacattgatatgaaccccgaggccctcaccgcgtggaaaagagccgcagccgccgtatacaggaaggataaagcgcgcaagtcccgacgcataagcctcgagtttatgctggaacaggccaacaagttcgccaaccacaaagctatctggttcccctacaacatggactggagagggagggtctacgccgtcagcatgttcaatccccagggcaacgacatgacgaagggccttctgacattggcaaaggggaagcctatcggaaaggaggggtactactggctcaagatccacggcgccaactgcgcgggagtggacaaggttccatttcccgagcgaattaagttcatcgaggaaaaccacgaaaacattatggcgtgcgctaaatcccccctcgagaacacatggtgggccgagcaagactccccgttctgttttttggcattctgctttgagtacgccggtgtgcagcaccatggcctctcatacaactgttccctgcccctggccttcgacggaagttgcagtgggattcaacatttcagcgcaatgttgcgggacgaggtcggtggcagggccgttaacctgctcccttccgaaacggtgcaggacatctacggaatcgtggcaaaaaaggtaaacgagatcctgcaagcggatgccatcaacgggacggacaatgaggtcgttacggtgacagacgaaaatactggggaaataagcgaaaaggtcaagctggggaccaaagcactcgcgggtcagtggctcgcctacggggtgacacgctccgtcaccaagagaagcgtgatgaccctcgcgtacggttcaaaagaattcggcttccgccagcaagtgctggaggacaccatccagccggcgattgactccgggaagggtctcatgtttacccagccgaaccaggccgcagggtacatggccaaactgatctgggaaagcgttagcgtcacagtggtcgccgcggttgaggcgatgaattggctgaagagcgcggcaaagctcctcgccgctgaggtgaaggacaaaaagaccggcgaaatcctgcgcaagcgctgcgccgtccactgggtcacgccggatggattccccgtctggcaggagtacaagaagcccatccaaacccggctcaacttgatgttccttggccagtttcgcctgcagcccacgataaacaccaacaaagacagcgagatcgacgcccacaagcaggagagcggcatcgcgcccaacttcgtgcacagtcaggacgggtcccatctgcggaaaactgttgtgtgggctcacgagaagtacggcattgagagcttcgccctgatacacgacagcttcgggaccataccagcggacgcagcgaacctgttcaaagccgtgcgggaaacaatggtcgacacctacgaaagctgcgacgtactggcagacttctatgaccaattcgccgaccagcttcacgagtcacagctcgacaagatgcccgctctgcccgcgaaaggcaacctgaatttgcgcgacatccttgagagcgattttgcgttcgcctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 47 - protein, 1138 aa, Artificial
  Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFASGGSPKKKRKV

SEQ ID NO: 48 - DNA, 7058 bp, Artificial
  Synthetic
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgagagccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtaacaccatcaacattgctaagaacgacttctcagacatagagctcgcggctattccgttcaacaccctggctgaccactacggcgagagactcgctagggagcagctggcgttggagcatgaatcctacgagatgggcgaggctaggttccgcaagatgttcgagcgacaattgaaggcaggggaggtggcggacaacgctgccgccaagcccctgatcacaaccttgctgcccaaaatgatcgcgcggatcaacgattggtttgaggaggttaaggcaaaacggggcaaacgcccgaccgcatttcaattcctccaagaaatcaagcctgaggctgttgcctacatcactatcaagacgacactggcgtgtctcacaagcgccgacaacaccaccgtgcaagccgtcgccagcgccatcgggcgggcaattgaggatgaggcacggtttggtaggatccgagacctggaagcgaagcacttcaagaagaacgtggaagagcagttgaacaaacgcgtcggccacgtgtataaaaaggctttcatgcaggtggtggaggccgatatgctcagtaaggggctgcttgggggggaggcgtggtcatcctggcacaaggaggatagcattcacgtgggggtccgatgtatcgagatgctgatagagagcaccggaatggtctccctccatcgccagaacgctggggtcgtagggcaggactccgagactattgagctggcccccgagtatgccgaagcaatcgctacacgcgcaggtgcactggctgggataagccctatgtttcagccctgcgtagtgcctccaaagccatggaccggcatcacagggggtggctattgggccaacggtaggcggcctctggccctggtacgcacgcacagcaagaaggcgctcatgcgctatgaagacgtttacatgcccgaggtttacaaggcgatcaatatcgcgcagaacaccgcctggaaaatcaataagaaggtgttggcggtcgcaaacgtgattaccaagtggaagcattgcccagtcgaggacatacccgccatagaacgcgaagagctgccgatgaagccggaagacattgatatgaaccccgaggccctcaccgcgtggaaaagagccgcagccgccgtatacaggaaggataaagcgcgcaagtcccgacgcataagcctcgagtttatgctggaacaggccaacaagttcgccaaccacaaagctatctggttcccctacaacatggactggagagggagggtctacgccgtcagcatgttcaatccccagggcaacgacatgacgaagggccttctgacattggcaaaggggaagcctatcggaaaggaggggtactactggctcaagatccacggcgccaactgcgcgggagtggacaaggttccatttcccgagcgaattaagttcatcgaggaaaaccacgaaaacattatggcgtgcgctaaatcccccctcgagaacacatggtgggccgagcaagactccccgttctgttttttggcattctgctttgagtacgccggtgtgcagcaccatggcctctcatacaactgttccctgcccctggccttcgacggaagttgcagtgggattcaacatttcagcgcaatgttgcgggacgaggtcggtggcagggccgttaacctgctcccttccgaaacggtgcaggacatctacggaatcgtggcaaaaaaggtaaacgagatcctgcaagcggatgccatcaacgggacggacaatgaggtcgttacggtgacagacgaaaatactggggaaataagcgaaaaggtcaagctggggaccaaagcactcgcgggtcagtggctcgcctacggggtgacacgctccgtcaccaagagaagcgtgatgaccctcgcgtacggttcaaaagaattcggcttccgccagcaagtgctggaggacaccatccagccggcgattgactccgggaagggtctcatgtttacccagccgaaccaggccgcagggtacatggccaaactgatctgggaaagcgttagcgtcacagtggtcgccgcggttgaggcgatgaattggctgaagagcgcggcaaagctcctcgccgctgaggtgaaggacaaaaagaccggcgaaatcctgcgcaagcgctgcgccgtccactgggtcacgccggatggattccccgtctggcaggagtacaagaagcccatccaaacccggctcaacttgatgttccttggccagtttcgcctgcagcccacgataaacaccaacaaagacagcgagatcgacgcccacaagcaggagagcggcatcgcgcccaacttcgtgcacagtcaggacgggtcccatctgcggaaaactgttgtgtgggctcacgagaagtacggcattgagagcttcgccctgatacacgacagcttcgggaccataccagcggacgcagcgaacctgttcaaagccgtgcgggaaacaatggtcgacacctacgaaagctgcgacgtactggcagacttctatgaccaattcgccgaccagcttcacgagtcacagctcgacaagatgcccgctctgcccgcgaaaggcaacctgaatttgcgcgacatccttgagagcgattttgcgttcgcctctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 49 - protein, 1225 aa, Artificial
  Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFASGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 50 - DNA, 43 bp, Artificial
  Synthetic
tgagagcgattttgcgttcgcctctggtggttctcccaagaag

SEQ ID NO: 51 - DNA, 45 bp, Artificial
  Synthetic
gttcttagcaatgttgatggtgttactttcgggtgtggcggactc

SEQ ID NO: 52 - DNA, 45 bp, Artificial
  Synthetic
gagtccgccacacccgaaagtaacaccatcaacattgctaagaac

SEQ ID NO: 53 - DNA, 41 bp, Artificial
  Synthetic
cttcttgggagaaccaccagaggcgaacgcaaaatcgctct

SEQ ID NO: 54 - DNA, 24 bp, Artificial
  Synthetic
tctggtggttctcccaagaagaag

SEQ ID NO: 55 - DNA, 18 bp, Artificial
  Synthetic
ggtggcggctctcgcggc

SEQ ID NO: 56 - DNA, 38 bp, Artificial
  Synthetic
cggccgcgagagccgccaccatggacagcctcttgatg

SEQ ID NO: 57 - DNA, 38 bp, Artificial
  Synthetic
ttcttgggagaaccaccagaagtacgaaatgcgtctcg

SEQ ID NO: 58 - DNA, 45 bp, Artificial
  Synthetic
agagcgattttgcgttcgcctctggtggttctactaatctgtcag

SEQ ID NO: 59 - DNA, 42 bp, Artificial
  Synthetic
gttcttagcaatgttgatggtgttactttcgggtgtggcgga

SEQ ID NO: 60 - DNA, 45 bp, Artificial; identical to SEQ ID NO: 52
  Synthetic

SEQ ID NO: 61 - DNA, 41 bp, Artificial
  Synthetic
cagattagtagaaccaccagaggcgaacgcaaaatcgctct

SEQ ID NO: 62 - DNA, 39 bp, Artificial
  Synthetic
tacgagacgcatttcgtactagcggcagcgagactcccg

SEQ ID NO: 63 - DNA, 42 bp, Artificial
  Synthetic
ggttcatcaagaggctgtccatggtggcggctctccctatag

SEQ ID NO: 64 - DNA, 41 bp, Artificial
  Synthetic
tatagggagagccgccaccatggacagcctcttgatgaacc

SEQ ID NO: 65 - DNA, 43 bp, Artificial
  Synthetic
ccgggagtctcgctgccgctagtacgaaatgcgtctcgtaagt

SEQ ID NO: 66 - DNA, 21 bp, Artificial
  Synthetic
tctggtggttctactaatctg

SEQ ID NO: 67 - DNA, 18 bp, Artificial
  Synthetic
actttcgggtgtggcgga

SEQ ID NO: 68 - DNA, 44 bp, Artificial
  Synthetic
agtccgccacacccgaaagtaacaccatcaacattgctaagaac

SEQ ID NO: 69 - DNA, 39 bp, Artificial
  Synthetic
agattagtagaaccaccagaggcgaacgcaaaatcgctc

SEQ ID NO: 70 - DNA, 18 bp, Artificial
  Synthetic
ttatgtttcagccctgcg

SEQ ID NO: 71 - DNA, 18 bp, Artificial
  Synthetic
actttcgggtgtggcgga

SEQ ID NO: 72 - DNA, 44 bp, Artificial; identical to SEQ ID NO: 68
  Synthetic

SEQ ID NO: 73 - DNA, 38 bp, Artificial
  Synthetic
tacgcagggctgaaacataaggcttatcccagccagtg

SEQ ID NO: 74 - DNA, 18 bp, Artificial
  Synthetic
ccttgagagcgattttgc

SEQ ID NO: 75 - DNA, 19 bp, Artificial
  Synthetic
ggatgggcttcttgtactc

SEQ ID NO: 76 - DNA, 40 bp, Artificial
  Synthetic
ggagtacaagaagcccatccgaacccggctcaacttgatg

SEQ ID NO: 77 - DNA, 38 bp, Artificial
  Synthetic
acgcaaaatcgctctcaaggatgtcgcgcaaattcagg

SEQ ID NO: 78 - DNA, 40 bp, Artificial
  Synthetic
attcgagctcggtacccgggtaatacgactcactataggc

SEQ ID NO: 79 - DNA, 38 bp, Artificial
  Synthetic
gccaagcttgcatgcctgcaagggaagaaagcgaaagg

SEQ ID NO: 80 - DNA, 27 bp, Artificial
  Synthetic
ccatcgatgagacccaagctggctagc

SEQ ID NO: 81 - DNA, 33 bp, Artificial
  Synthetic
ccatcgatatttcgataagccagtaagcagtgg

SEQ ID NO: 82 - DNA, 28 bp, Artificial
  Synthetic
tgaattaattaagaattatcaccgcttc

SEQ ID NO: 83 - DNA, 18 bp, Artificial
  Synthetic
ctagtggatccgagctcg

SEQ ID NO: 84 - DNA, 38 bp, Artificial
  Synthetic
accgagctcggatccactagatggtgagcaagggcgag

SEQ ID NO: 85 - DNA, 43 bp, Artificial
  Synthetic
tgataattcttaattaattcattacttgtacagctcgtccatg

SEQ ID NO: 86 - DNA, 19 bp, Artificial
  Synthetic
aattcgaagcttgagctcg

SEQ ID NO: 87 - DNA, 19 bp, Artificial
  Synthetic
actagttctagagtcggtg

SEQ ID NO: 88 - DNA, 40 bp, Artificial
  Synthetic
acaccgactctagaactagttaatacgactcactataggg

SEQ ID NO: 89 - DNA, 42 bp, Artificial
  Synthetic
tcgagctcaagcttcgaatttttattaggaaaacaacagatg

SEQ ID NO: 90 - DNA, 20 bp, Artificial
  Synthetic
atggtgagcaagggcgagga

SEQ ID NO: 91 - DNA, 23 bp, Artificial
  Synthetic
ttacttgtacagctcgtccatgc

SEQ ID NO: 92 - DNA, 19 bp, Artificial
  Synthetic
gcaaatgggcggtaggcgt

SEQ ID NO: 93 - DNA, 19 bp, Artificial
  Synthetic
ggcgctggcaagtgtagcg

SEQ ID NO: 94 - DNA, 24 bp, Artificial
  Synthetic
aactagagaacccactgcttactg

SEQ ID NO: 95 - DNA, 19 bp, Artificial
  Synthetic
ggcgctggcaagtgtagcg

SEQ ID NO: 96 - DNA, 18 bp, Artificial
  Synthetic
tcagacaacctcatttcc

SEQ ID NO: 97 - DNA, 23 bp, Artificial
  Synthetic
gcttactacaacttttaaaagtt

SEQ ID NO: 98 - DNA, 20 bp, Artificial
  Synthetic
tcaccagtcgtttttcagat

SEQ ID NO: 99 - DNA, 29 bp, Artificial
  Synthetic
ccatactccttttaaaaatataatacaac

SEQ ID NO: 100 - DNA, 19 bp, Artificial
  Synthetic
gatcttcagacctggagga

SEQ ID NO: 101 - DNA, 18 bp, Artificial
  Synthetic
tagaaggcacagtcgagg

SEQ ID NO: 102 - DNA, 20 bp, Artificial
  Synthetic
gaacagggacttgaaagcga

SEQ ID NO: 103 - DNA, 18 bp, Artificial
  Synthetic
tagaaggcacagtcgagg

SEQ ID NO: 104 - DNA, 60 bp, Artificial
  Synthetic
aagcgcgatcacatggtcctgctggagttcgtgaccgccgccggatcactctcggcatgg

SEQ ID NO: 105 - DNA, 61 bp, Artificial
  Synthetic
aagcgcgatcacatggtcctgctggagttcgtgaccgccgccgggatcactctcggcatgg

SEQ ID NO: 106 - DNA, 52 bp, Artificial
  Synthetic
aagcgcgatcacatggtgttcgtgaccgccgccgggatcactctcggcatgg

SEQ ID NO: 107 - DNA, 51 bp, Artificial
  Synthetic
aagcgcgatcacatgagttcgtgaccgccgccgggatcactctcggcatgg

SEQ ID NO: 108 - DNA, 61 bp, Artificial
  Synthetic
  misc_feature (35)..(35): n is a, c, g, or t
  misc_feature (38)..(38): n is a, c, g, or t
  misc_feature (47)..(47): n is a, c, g, or t
  misc_feature (58)..(58): n is a, c, g, or t
aagcgcgatcacatggtcctgctggagttcgtgancgncgccgggancactctcggcntgg

SEQ ID NO: 109 - DNA, 61 bp, Artificial
  Synthetic
  misc_feature (18)..(18): n is a, c, g, or t
  misc_feature (57)..(57): n is a, c, g, or t
aagcgcgatcacatggtnctgctggagttcgtgaccgccgccgggatcactctcggnatgg

SEQ ID NO: 110 - DNA, 59 bp, Artificial
  Synthetic
aagcgcgatcacatggtcctgctggagttcgtgaccgccgccgggatcactctcggcat

SEQ ID NO: 111 - DNA, 54 bp, Artificial
  Synthetic
atcacatggtcctgctggagttcgtgaccgccgccgggatcactctcggcatgg

SEQ ID NO: 112 - DNA, 51 bp, Artificial
  Synthetic
aagcgcgatcacatggtcctgctgccgccgccgggatcactctcggcatgg

SEQ ID NO: 113 - DNA, 51 bp, Artificial; identical to SEQ ID NO: 107
  Synthetic

SEQ ID NO: 114 - DNA, 51 bp, Artificial; identical to SEQ ID NO: 112
  Synthetic

SEQ ID NO: 115 - DNA, 51 bp, Artificial; identical to SEQ ID NO: 112
  Synthetic

SEQ ID NO: 116 - DNA, 48 bp, Artificial
  Synthetic
aagcgcgactgctggagttcgtgaccgccgccgggatcactctcggca

SEQ ID NO: 117 - DNA, 56 bp, Artificial
  Synthetic
aagcgcgatcacatggtcctgctggagttccgccgccgggatcactctcggcatgg

SEQ ID NO: 118 - DNA, 52 bp, Artificial
  Synthetic
aagcgcgatcacatggtcttcgtgaccgccgccgggatcactctcggcatgg

SEQ ID NO: 119 - DNA, 52 bp, Artificial; identical to SEQ ID NO: 118
  Synthetic

SEQ ID NO: 120 - DNA, 51 bp, Artificial
  Synthetic
aagcgcgatcgctggagttcgtgaccgccgccgggatcactctcggcatgg

SEQ ID NO: 121 - DNA, 51 bp, Artificial; identical to SEQ ID NO: 120
  Synthetic

SEQ ID NO: 122 - DNA, 61 bp, Artificial; identical to SEQ ID NO: 105
  Synthetic

SEQ ID NO: 123 - DNA, 56 bp, Artificial; identical to SEQ ID NO: 117
  Synthetic

SEQ ID NO: 124 - DNA, 51 bp, Artificial; identical to SEQ ID NO: 112
  Synthetic

SEQ ID NO: 125 - DNA, 61 bp, Artificial; identical to SEQ ID NO: 105
  Synthetic

SEQ ID NO: 126 - DNA, 50 bp, Artificial
  Synthetic
aagcgcgatcacatggtcctgctggagttcgtgaccgccgccgggattgg

SEQ ID NO: 127 - DNA, 50 bp, Artificial
  Synthetic
  misc_feature (28)..(29): n is a, c, g, or t
  misc_feature (32)..(32): n is a, c, g, or t
  misc_feature (43)..(43): n is a, c, g, or t
  misc_feature (46)..(47): n is a, c, g, or t
aagcgcgatcacatggtcctgctggagnncgngaccgccgccnggnntgg

SEQ ID NO: 128 - DNA, 51 bp, Artificial; identical to SEQ ID NO: 112
  Synthetic

SEQ ID NO: 129 - DNA, 61 bp, Artificial; identical to SEQ ID NO: 105
  Synthetic

SEQ ID NO: 130 - DNA, 60 bp, Artificial
  Synthetic
  misc_feature (34)..(36): n is a, c, g, or t
aagcgcgatcacatggtcctgctggagttcgtgnnnccgccgggatcactctcggcatgg

SEQ ID NO: 131 - DNA, 60 bp, Artificial
  Synthetic
aagcgcgatcacatggtctgctggagttcgtgaccgccgccgggatcactctcggcatgg

SEQ ID NO: 132 - DNA, 61 bp, Artificial; identical to SEQ ID NO: 105
  Synthetic

SEQ ID NO: 133 - DNA, 60 bp, Artificial; identical to SEQ ID NO: 131
  Synthetic

SEQ ID NO: 134 - DNA, 61 bp, Artificial
  Synthetic
  misc_feature (32)..(32): n is a, c, g, or t
  misc_feature (40)..(40): n is a, c, g, or t
  misc_feature (44)..(44): n is a, c, g, or t
  misc_feature (54)..(54): n is a, c, g, or t
aagcgcgatcacatggtcctgctggagttcgngaccgccnccgngatcactctnggcatgg

SEQ ID NO: 135 - DNA, 61 bp, Artificial; identical to SEQ ID NO: 105
  Synthetic

SEQ ID NO: 136 - DNA, 61 bp, Artificial; identical to SEQ ID NO: 105
  Synthetic

SEQ ID NO: 137 - DNA, 60 bp, Artificial
  Synthetic
aagcgcgatcacatggtcctgctggagttcgtaccgccgccgggatcactctcggcatgg

SEQ ID NO: 138 - DNA, 60 bp, Artificial; identical to SEQ ID NO: 131
  Synthetic

SEQ ID NO: 139 - DNA, 60 bp, Artificial
  Synthetic
  misc_feature (54)..(55): n is a, c, g, or t
aagcgcgatcacatggtctactagagttcatgaccgccgccgggatcactctcnncatgg

SEQ ID NO: 140 - DNA, 60 bp, Artificial; identical to SEQ ID NO: 131
  Synthetic

SEQ ID NO: 141 - DNA, 60 bp, Artificial; identical to SEQ ID NO: 131
  Synthetic

SEQ ID NO: 142 - DNA, 60 bp, Artificial; identical to SEQ ID NO: 131
  Synthetic

SEQ ID NO: 143 - DNA, 60 bp, Artificial
  Synthetic
  misc_feature (5)..(5): n is a, c, g, or t
aagcncgatcacatggtctgctggagttcgtgaccgccgccgggatcactctcggcatgg

SEQ ID NO: 144 - DNA, 61 bp, Artificial
  Synthetic
  misc_feature (26)..(26): n is a, c, g, or t
aagcgcgatcacatggtcctgctggngttcgtgaccgccgccgggatcactctcggcatgg

SEQ ID NO: 145 - DNA, 60 bp, Artificial
  Synthetic
aagcgcgatcacatggtcctgctgagttcgtgaccgccgccgggatcactctcggcatgg

SEQ ID NO: 146 - DNA, 60 bp, Artificial; identical to SEQ ID NO: 137
  Synthetic

SEQ ID NO: 147 - DNA, 61 bp, Artificial; identical to SEQ ID NO: 105
  Synthetic

SEQ ID NO: 148 - DNA, 60 bp, Artificial
  Synthetic
aagtgcgatcacatggtctgctggagttcgtgaccgccgccgggatcactctcggcatgg

SEQ ID NO: 149 - DNA, 61 bp, Artificial; identical to SEQ ID NO: 105
  Synthetic

SEQ ID NO: 150 - DNA, 61 bp, Artificial
  Synthetic
aagcgcgatcacatggtcctgctggagttcgtgaccgccgccgggatcactctcgacatgg

SEQ ID NO: 151 - DNA, 59 bp, Artificial
  Synthetic
aagcgcgatcacatgtcctgctggagttcgtgaccgccgccgggatcactctcggcagg

SEQ ID NO: 152 - DNA, 61 bp, Artificial
  Synthetic
  misc_feature (47)..(47): n is a, c, g, or t
  misc_feature (57)..(57): n is a, c, g, or t
aagcgcgatcacatggtcctgctggagttcgtgaccgccgccgggancactctcggnatgg

SEQ ID NO: 153 - DNA, 60 bp, Artificial; identical to SEQ ID NO: 131
  Synthetic

SEQ ID NO: 154 - DNA, 60 bp, Artificial
  Synthetic
  misc_feature (22)..(23): n is a, c, g, or t
  misc_feature (26)..(26): n is a, c, g, or t
  misc_feature (34)..(34): n is a, c, g, or t
aagcgcgatcacatggtctgcnnganttcgtgancgccgccgggatcactctcggcatgg

SEQ ID NO: 155 - DNA, 55 bp, Artificial
  Synthetic
aagcgcgatcacatggtcctactagagtccgccgccgggatcactctcggcatgg

SEQ ID NO: 156 - DNA, 55 bp, Artificial
  Synthetic
aaacacgatcacatggtcctactggagtccgccgccgggatcactctcggcatgg

SEQ ID NO: 157 - DNA, 55 bp, Artificial
  Synthetic
  misc_feature (10)..(10): n is a, c, g, or t
  misc_feature (27)..(28): n is a, c, g, or t
aagcgcgatnacatggtcctgctggannccgccgccgggatcactctcggcatgg

SEQ ID NO: 158 - DNA, 56 bp, Artificial; identical to SEQ ID NO: 117
  Synthetic

SEQ ID NO: 159 - DNA, 56 bp, Artificial; identical to SEQ ID NO: 117
  Synthetic

SEQ ID NO: 160 - DNA, 55 bp, Artificial
  Synthetic
  misc_feature (38)..(38): n is a, c, g, or t
  misc_feature (47)..(47): n is a, c, g, or t
aagcgcgatcacatggtcctactagagtccgccgccgngatcactcncggcatgg

SEQ ID NO: 161 - DNA, 60 bp, Artificial
  Synthetic
  misc_feature (21)..(21): n is a, c, g, or t
aagcgcgatcacatggtcctnctggagttcgtgaccgccgccgggatcactccggcatgg

SEQ ID NO: 162 - DNA, 60 bp, Artificial
  Synthetic
aaacgcgatcacatggtcctgctggagttcgtgaccgccgccgggatcactccggcatgg

SEQ ID NO: 163 - DNA, 60 bp, Artificial; identical to SEQ ID NO: 162
  Synthetic

SEQ ID NO: 164 - DNA, 60 bp, Artificial
  Synthetic
  misc_feature (3)..(3): n is a, c, g, or t
  misc_feature (15)..(15): n is a, c, g, or t
  misc_feature (23)..(23): n is a, c, g, or t
  misc_feature (34)..(34): n is a, c, g, or t
  misc_feature (40)..(40): n is a, c, g, or t
aancgcgatcacatngtcctgcnggagttcgtgnccgccnccgggatcattccggcatgg

SEQ ID NO: 165 - DNA, 60 bp, Artificial
  Synthetic
  misc_feature (46)..(46): n is a, c, g, or t
aagcgcgatcacatggtcctgctggagttcgtgaccgccgccgggntcattccggcatgg

SEQ ID NO: 166 - DNA, 56 bp, Artificial
  Synthetic
aagcgcgatcacatggtcctgctggagttcgtgaccgccgccgggatcactcttgg

SEQ ID NO: 167 - DNA, 56 bp, Artificial; identical to SEQ ID NO: 166
  Synthetic

SEQ ID NO: 168 - DNA, 56 bp, Artificial
  Synthetic
aaacacgatcacatggtcctactagagttcgtgaccgccgccgggatcactcttgg

SEQ ID NO: 169 - DNA, 55 bp, Artificial
  Synthetic
aaacacgatcacatggtcctactagagtccgccgccgggatcactctcggcatgg

SEQ ID NO: 170 - DNA, 61 bp, Artificial; identical to SEQ ID NO: 105
  Synthetic

SEQ ID NO: 171 - DNA, 53 bp, Artificial
  Synthetic
  misc_feature (38)..(39): n is a, c, g, or t
  misc_feature (42)..(42): n is a, c, g, or t
aagcgcgatcacatggtcctgctggagttcgtgacggnncantctcggcatgg

SEQ ID NO: 172 - DNA, 53 bp, Artificial
  Synthetic
aagcgcgatcacatggtcctgctggagttcgtgacggatcactctcggcatgg

SEQ ID NO: 173 - DNA, 53 bp, Artificial; identical to SEQ ID NO: 172
  Synthetic

SEQ ID NO: 174 - DNA, 53 bp, Artificial; identical to SEQ ID NO: 172
  Synthetic

SEQ ID NO: 175 - DNA, 60 bp, Artificial
  Synthetic
  misc_feature (20)..(20): n is a, c, g, or t
aagcgcgatcacatggtccngctggagttcgtgaccgccgccgggatcactccggcatgg

SEQ ID NO: 176 - DNA, 60 bp, Artificial
  Synthetic
aagcgcgatcacatggtcctgctggagttcgtgaccgccgccgggatcactccggcatgg

SEQ ID NO: 177 - DNA, 56 bp, Artificial; identical to SEQ ID NO: 168
  Synthetic

SEQ ID NO: 178 - DNA, 56 bp, Artificial; identical to SEQ ID NO: 168
  Synthetic

SEQ ID NO: 179 - DNA, 56 bp, Artificial
  Synthetic
aaacgcgatcacatggtcctgctggagttcgtgaccgccgccgggatcattcttgg

SEQ ID NO: 180 - DNA, 23 bp, Artificial
  Synthetic
taatacgactcactatagggaga

SEQ ID NO: 181 - DNA, 23 bp, Artificial
  Synthetic
taatacaactcactatagggaga

SEQ ID NO: 182 - DNA, 30 bp, Artificial
  Synthetic
gtgaccaccctgacccacggcgtgcagtgc

SEQ ID NO: 183 - DNA, 29 bp, Artificial
  Synthetic
gtaccaccctgacctacggcgtgcagtgc

(19) United States
(12) Patent Application Publication
(10) Pub. No.: US 2022/0170006 A1
(43) Pub. Date: Jun. 2, 2022

(54) A PSEUDO-RANDOM DNA EDITOR FOR EFFICIENT AND CONTINUOUS NUCLEOTIDE DIVERSIFICATION IN HUMAN CELLS

(71) Applicants: THE BROAD INSTITUTE, INC., Cambridge, MA (US); PRESIDENT AND FELLOWS OF HARVARD COLLEGE, Cambridge, MA (US)

(72) Inventors: Fei Chen, Cambridge, MA (US); Haiqi Chen, Cambridge, MA (US); Sophia Liu, Cambridge, MA (US); Samuel Padula, Cambridge, MA (US); Kettner Griswold, Cambridge, MA (US)

(73) Assignees: THE BROAD INSTITUTE, INC., Cambridge, MA (US); PRESIDENT AND FELLOWS OF HARVARD COLLEGE, Cambridge, MA (US)

(21) Appl. No.: 17/601,290

(22) PCT Filed: Apr. 3, 2020
(86) PCT No.: PCT/US2020/026679

§ 371 Date: Oct. 4, 2021

Related U.S. Application Data
(60) Provisional application No. 62/830,084, filed on Apr. 5, 2019.

(51) Int. Cl. C12N 9/78; C12N 9/12; C12N 15/10

(52) U.S. Cl. CPC C12N 9/78; C12N 9/1247; C07K 2319/09; C12N 15/1024; C12Y 305/04001; C12Y 207/07006

(57) ABSTRACT

The present disclosure provides compositions and methods for performance of targeted mutagenesis in higher eukaryotic cells, e.g., mammalian cells, across large stretches of targeted sequence. Compositions and methods that rely upon combination of a bacteriophage polymerase with a nucleic acid-editing deaminase to achieve robust mutagenesis of targeted regions of nucleic acid sequence under control of a phage promoter are specifically provided.

### CROSS REFERENCE TO RELATED APPLICATIONS

This application claims the benefit of U.S. Provisional Application No. 62/830,084 filed Apr. 5, 2019, entitled “A Pseudo-Random DNA Editor for Efficient and Continuous Nucleotide Diversification in Human Cells,” the entire contents of which are incorporated herein by reference.

### STATEMENT REGARDING FEDERALLY SPONSORED RESEARCH

This invention was made with government support under Grant No. 1DP50D024583 awarded by the National Institutes of Health. The government has certain rights in the invention.

### FIELD OF THE INVENTION

The invention relates generally to methods of DNA editing capable of providing efficient and continuous nucleotide diversification in human cells.

### BACKGROUND OF THE INVENTION

The advancement of methods for studying the genetic dynamics of eukaryotic cells, such as directed evolution, lineage tracing, and molecular recording, depends upon development of additional tools for targeted, continuous mutagenesis. Existing tools tend to rely upon non-physiological environments, tend to saturate mutagenized sites rapidly, and/or have only been adapted in bacterial or yeast systems. While approaches for relatively long editing regions have been identified and demonstrated in bacterial and yeast cells, a need exists for an editor system that is efficient in inducing continuous nucleotide diversification in cells of multicellular eukaryotic organisms, especially in mammalian cells.

### BRIEF SUMMARY OF THE INVENTION

The current disclosure relates, at least in part, to the discovery of compositions and methods capable of performing targeted mutagenesis in higher eukaryotic cells, particularly in mammalian cells in culture, across large spans of targeted nucleic acid sequence, at mutation rates that are robust as compared to background rates of polymerase-mediated mutation. In certain aspects, the compositions and methods of the instant disclosure provide for enhanced, targeted mutagenesis of mammalian cells capable of enabling directed evolution of targeted sequences in living cells. Accordingly, application of the instant compositions and methods to drug and/or peptide evolution and screening in mammalian cell lines is expressly contemplated, as are other applications as set forth herein and as known in the art.

In one aspect, the instant disclosure provides a fusion protein that includes: (i) a bacteriophage RNA polymerase and (ii) a nucleic acid-editing deaminase.

In one embodiment, the bacteriophage RNA polymerase is a T7 RNA polymerase or a T7-like RNA polymerase. Optionally, the T7-like RNA polymerase is a N4 RNA polymerase.

In another embodiment, the nucleic acid-editing deaminase is a cytidine deaminase, an adenine deaminase and/or a guanine deaminase. Optionally, the cytidine deaminase is an activation-induced cytidine deaminase. Optionally, the activation-induced cytidine deaminase is rat APOBEC1 or AID. Optionally, the AID cytidine deaminase is a hyperactive mutant of AID. Optionally, the hyperactive mutant of AID is AID*Δ.

In an additional embodiment, the fusion protein further includes a nuclear localization signal (NLS). Optionally, the NLS is attached at the C-terminus of the fusion protein.

In certain embodiments, the fusion protein further includes a uracil glycosylase inhibitor (UGI). Optionally, the UGI is attached at a location C-terminal to the nucleic acid-editing deaminase and the bacteriophage RNA polymerase.

Another aspect of the instant disclosure provides a nucleic acid that includes: (i) a nucleic acid sequence encoding for a bacteriophage RNA polymerase and (ii) a nucleic acid sequence encoding for a nucleic acid-editing deaminase.

In one embodiment, the nucleic acid further includes a nucleic acid sequence encoding for a nuclear localization signal (NLS). Optionally, nucleic acid sequence encoding for the NLS is attached at the 3′-terminus of the nucleic acid.

In another embodiment, the nucleic acid further includes a nucleic acid sequence encoding for a uracil glycosylase inhibitor (UGI). Optionally, the nucleic acid sequence encoding for the UGI is attached at a location 3′ of the nucleic acid sequence encoding for the nucleic acid-editing deaminase and the nucleic acid sequence encoding for the bacteriophage RNA polymerase.

In an additional embodiment, the nucleic acid further includes a mammalian expression vector promoter. Optionally, the mammalian expression vector promoter is located 5′ of the nucleic acid sequence encoding for a bacteriophage RNA polymerase and the nucleic acid sequence encoding for the nucleic acid-editing deaminase. Optionally, the mammalian expression vector promoter is a CMV promoter, a SV-40 promoter, an (EF)-1 promoter or a tetracycline-inducible mammalian promoter (e.g., Tet-On, Tet-Off, etc.).

In another embodiment, the nucleic acid further includes an origin of replication. Optionally, the nucleic acid is a plasmid.

An additional aspect of the disclosure provides a mammalian cell that includes a first nucleic acid of the disclosure (e.g., encoding for a fusion protein that includes a bacteriophage RNA polymerase and a nucleic acid-editing deaminase).

In one embodiment, the mammalian cell further harbors a second nucleic acid that includes a bacteriophage promoter corresponding to the bacteriophage RNA polymerase of the first nucleic acid. Optionally, the bacteriophage promoter is a T7 promoter or is a T7-like promoter. Optionally, the T7-like promoter is a N4 promoter.

In certain embodiments, the bacteriophage promoter of the second nucleic acid is operably linked to a target nucleic acid sequence. Optionally, the target nucleic acid sequence is a mammalian target nucleic acid sequence. Optionally, the mammalian target nucleic acid sequence is ABL1, FLT3, MCL1, PRKCQ, WEE1, ABL2, FNTA, MDM2, PRKCSH, XIAP, AKT1, GSK3A, MEK1, PRKCZ, AKT2, GSK3B, MET, PRKDC, AKT3, HDAC1, MTOR, PSENEN, AIX, HDAC2, NFKB1, PSMB5, AR, HDAC3, NTRK1, PTK2, ATM, HDAC6, P4HB, PTPN11, AURKA, HDAC8, p53, PTPN6, AURKB, HER2, PAK1, RAC1, AURKC, HSP90AA1, PARP1, RET, BCL2, HSP90AB1, PDGFRA, ROCK1, BCL-ABL1, HSP90AB4P, PDGFRB, ROCK2, BMX HSP90B1, PDK1, RPS6KA1, BRAF, HSP90B3P, PIK3CA, RPS6KA2, BTK, IGF1R, PIK3CB, RPS6KA3, CASP3, IKBKE, PIK3CD, RPS6KA4, CCR5, ITK, PIK3CG, RPS6KA5, CDK1, JAK2, PLK1, RPS6KA6, CDK2, KDR, PLK2, RPS6KB2, CDK4, KIT, PLK3, RXRA, CDK6, KRAS, PPM1D, RXRB, CDK7, MAP2K1, PRKAA1, SGK3, CTNNB1, MAP2K2, PRKCA, SMO, DHFR, MAPK11, PRKCB, SRC, EGFR, MAPK12, PRKCD, SYK, ERBB2, MAPK13, PRKCE, TBK1, FGFR1, MAPK14, PRKCG, TEC, FGFR3, MAPK7, PRKCH, TNF, FLT1, MAPK8, PRKCI and/or TOP1.

In some embodiments, the second nucleic acid is harbored on a plasmid within the mammalian cell.

In an embodiment, the second nucleic acid is integrated into the genome of the mammalian cell. Optionally, the second nucleic acid is integrated into the genome of the mammalian cell at the Rosa 26 locus. Optionally, the first nucleic acid and the second nucleic acid are integrated into the genome of the mammalian cell at the Rosa 26 locus.

In embodiments, the mammalian cell is a mouse cell. Optionally, the mammalian cell is a mouse oocyte cell.

In certain embodiments, the mammalian cell further harbors a cell type-specific Cre-recombinase or Cre-ER capable of inducing conditional expression of the first nucleic acid and/or the second nucleic acid where Cre-recombinase is present.

In one embodiment, the mammalian cell is a cell of a mammalian cell line. Optionally, the mammal cell line is HEK293T, VERO, BHK, HeLa, CV1, MDCK, 3T3, a myeloma cell line, PC12, WI38 or Chinese hamster ovary (CHO).

Another aspect of the instant disclosure provides a method for performing mutagenesis upon a target nucleic acid of a mammalian cell, the method involving: (a) providing a mammalian cell; (b) contacting the mammalian cell with: (i) a first nucleic acid of the instant disclosure; and (ii) a second nucleic acid that includes a bacteriophage promoter operably linked to a target nucleic acid; where contacting of the mammalian cell with the first nucleic acid and the second nucleic acid is performed in any order, including concurrently; and (c) culturing the mammalian cell for a duration of time sufficient for mutation of the target nucleic acid to be detected.

In one embodiment, the first nucleic acid is harbored on a plasmid.

In another embodiment, contacting step (b) includes transfecting the first nucleic acid into the mammalian cell. Optionally, the transfecting involves a lentivirus.

In other embodiments, contacting step (b) includes genomic integration of the first nucleic acid.

In certain embodiments, the second nucleic acid is harbored on a plasmid.

In an additional embodiment, contacting step (b) involves transfecting the second nucleic acid into the mammalian cell.

In other embodiments, contacting step (b) involves genomic integration of the second nucleic acid.

A further aspect of the instant disclosure provides a kit that includes a nucleic acid of the instant disclosure and instructions for its use.

In one embodiment, the kit further includes a transfection agent. Optionally, the transfection agent is a lentivirus.

### Definitions

As used herein, the term “bacteriophage RNA polymerase” refers to any bacteriophage-derived RNA polymerase (RNAP) that possesses DNA processivity, which is expressly contemplated to include all variant, mutant and/or derivative forms of bacteriophage RNAP, provided that DNA processivity is maintained. Specific examples of RNAP are set forth below, and include, without limitation, T7 RNAP and T7-like RNA polymerases, such as T3 RNAP, SP6 RNAP and/or N4 RNAP.

The term “nucleic acid-editing deaminase,” as used herein, refers to any deaminase that is capable of performing somatic hypermutation. Deaminases effect the deamination or removal of an amine group of a nucleic acid. Expressly contemplated examples of nucleic acid-editing deaminases include, but are not limited to, adenine deaminase, cytidine deaminase (including activation-induced cytidine deaminase), and guanine deaminase. Specific examples of nucleic acid-editing deaminases are provided in additional detail elsewhere herein.

The term “fusion protein” as used herein refers to an engineered polypeptide that combines sequence elements excerpted from two or more other proteins, optionally from two or more naturally-occurring proteins.

The terms “transfect,” “transfects,” “transfecting” and “transfection” as used herein refer to the delivery of nucleic acids (usually DNA or RNA) to the cytoplasm or nucleus of cells, e.g., through the use of lentiviral delivery vectors/plasmids, cationic lipid vehicle(s) and/or by means of electroporation, or other art-recognized means of transfection.

The term “plasmid” as used herein refers to a construction comprised of genetic material designed to direct transformation of a targeted cell. The plasmid consist of a plasmid backbone. A “plasmid backbone” as used herein contains multiple genetic elements positional and sequentially oriented with other necessary genetic elements such that the nucleic acid in a nucleic acid cassette can be transcribed and when necessary translated in the transfected cells. The term plasmid as used herein can refer to nucleic acid, e.g., DNA derived from a plasmid vector, cosmid, phagemid or bacteriophage, into which one or more fragments of nucleic acid may be inserted or cloned which encode for particular genes

A “viral vector” as used herein is one that is physically incorporated in a viral particle by the inclusion of a portion of a viral genome within the vector, e.g., a packaging signal, and is not merely DNA or a located gene taken from a portion of a viral nucleic acid. Thus, while a portion of a viral genome can be present in a plasmid of the present disclosure, that portion does not cause incorporation of the plasmid into a viral particle and thus is unable to produce an infective viral particle.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the term “integrating vector” refers to a vector whose integration or insertion into a nucleic acid (e.g., a chromosome) is accomplished via an integrase. Examples of “integrating vectors” include, but are not limited to, retroviral vectors, transposons, and adeno associated virus vectors.

As used herein, the term “integrated” refers to a vector that is stably inserted into the genome (i.e., into a chromosome) of a host cell.

As used herein, the term “genome” refers to the genetic material (e.g., chromosomes) of an organism.

The term “target nucleic acid” refers to any nucleotide sequence (e.g., RNA or DNA), the manipulation of which may be deemed desirable for any reason (e.g., for directed evolution, to treat disease, confer improved qualities, expression of a protein of interest in a host cell, expression of a ribozyme, etc.), by one of ordinary skill in the art. Such nucleic acid sequences include, but are not limited to, coding sequences of genes (e.g., enzyme-encoding genes, transcription factor-encoding genes, cytokine-encoding genes, reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

As used herein, the term “exogenous gene” refers to a gene that is not naturally present in a host organism or cell, or is artificially introduced into a host organism or cell.

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

As used herein, the term “variant,” when used in reference to a protein, refers to proteins encoded by partially homologous nucleic acids so that the amino acid sequence of the proteins varies. As used herein, the term “variant” encompasses proteins encoded by homologous genes having both conservative and nonconservative amino acid substitutions that do not result in a change in protein function, as well as proteins encoded by homologous genes having amino acid substitutions that cause decreased (e.g., null mutations) protein function or increased protein function.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “regulatory element” refers to a genetic element which controls some aspect of the expression of nucleic acid sequences. For example, a promoter is a regulatory element that facilitates the initiation of transcription of an operably linked coding region. Other regulatory elements are splicing signals, polyadenylation signals, termination signals, RNA export elements, internal ribosome entry sites, etc.

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and Maniatis et al., supra). For example, the SV40 early gene enhancer is very active in a wide variety of cell types from many mammalian species and has been widely used for the expression of proteins in mammalian cells (Dijkema et al, EMBO J. 4:761 [1985]). Two other examples of promoter/enhancer elements active in a broad range of mammalian cell types are those from the human elongation factor 1α gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques such as cloning and recombination) such that transcription of that gene is directed by the linked enhancer/promoter.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” promoter is one which is capable of directing a level of transcription of an operably linked nucleic acid sequence in the presence of a stimulus (e.g., heat shock, chemicals, etc.) which is different from the level of transcription of the operably linked nucleic acid sequence in the absence of the stimulus.

Eukaryotic expression vectors may also contain “viral replicons” or “viral origins of replication.” Viral replicons are viral DNA sequences that allow for the extrachromosomal replication of a vector in a host cell expressing the appropriate replication factors. Vectors that contain either the SV40 or polyoma virus origin of replication replicate to high “copy number” (up to 104 copies/cell) in cells that express the appropriate viral T antigen. Vectors that contain the replicons from bovine papillomavirus or Epstein-Barr virus replicate extrachromosomally at “low copy number” (^{˜}100 copies/cell). However, it is not intended that expression vectors be limited to any particular viral origin of replication.

As used herein, the term “retrovirus” refers to a retroviral particle which is capable of entering a cell (i.e., the particle contains a membrane-associated protein such as an envelope protein or a viral G glycoprotein which can bind to the host cell surface and facilitate entry of the viral particle into the cytoplasm of the host cell) and integrating the retroviral genome (as a doublc-stranded provirus) into the genome of the host cell. The term “retrovirus” encompasses Oncovirinae (e.g., Moloney murine leukemia virus (MoMOLV), Moloney murine sarcoma virus (MoMSV), and Mouse mammary tumor virus (MMTV), Spumavirinae, amd Lentivirinae (e.g., Human immunodeficiency virus, Simian immunodeficiency virus, Equine infection anemia virus, and Caprine arthritis-encephalitis virus; See, e.g., U.S. Pat. Nos. 5,994,136 and 6,013,516, both of which are incorporated herein by reference).

As used herein, the term “retroviral vector” refers to a retrovirus that has been modified to express a gene of interest. Retroviral vectors can be used to transfer genes efficiently into host cells by exploiting the viral infectious process. Foreign or heterologous genes cloned (i.e., inserted using molecular biological techniques) into the retroviral genome can be delivered efficiently to host cells which are susceptible to infection by the retrovirus.

The term “Rhabdoviridae” refers to a family of enveloped RNA viruses that infect animals, including humans, and plants. The Rhabdoviridae family encompasses the genus Vesiculovirus which includes vesicular stomatitis virus (VSV), Cocal virus, Piry virus, Chandipura virus, and Spring viremia of carp virus (sequences encoding the Spring viremia of carp virus are available under GenBank accession number U18101). The G proteins of viruses in the Vesiculovirus genera are virally-encoded integral membrane proteins that form externally projecting homotrimeric spike glycoproteins complexes that are required for receptor binding and membrane fusion. The G proteins of viruses in the Vesiculovirus genera have a covalently bound palmititic acid (C16) moiety. The amino acid sequences of the G proteins from the Vesiculoviruses are fairly well conserved. For example, the Piry virus G protein share about 38% identity and about 55% similarity with the VSV G proteins (several strains of VSV are known, e.g., Indiana, New Jersey, Orsay, San Juan, etc., and their G proteins are highly homologous). The Chandipura virus G protein and the VSV G proteins share about 37% identity and 52% similarity. Given the high degree of conservation (amino acid sequence) and the related functional characteristics (e.g., binding of the virus to the host cell and fusion of membranes, including syncytia formation) of the G proteins of the Vesiculoviruses, the G proteins from non-VSV Vesiculoviruses may be used in place of the VSV G protein for the pseudotyping of viral particles. The G proteins of the Lyssa viruses (another genera within the Rhabdoviridae family) also share a fair degree of conservation with the VSV G proteins and function in a similar manner (e.g., mediate fusion of membranes) and therefore may be used in place of the VSV G protein for the pseudotyping of viral particles. The Lyssa viruses include the Mokola virus and the Rabies viruses (several strains of Rabies virus are known and their G proteins have been cloned and sequenced). The Mokola virus G protein shares stretches of homology (particularly over the extracellular and transmembrane domains) with the VSV G proteins which show about 31% identity and 48% similarity with the VSV G proteins. Preferred G proteins share at least 25% identity, preferably at least 30% identity and most preferably at least 35% identity with the VSV G proteins. The VSV G protein from which New Jersey strain (the sequence of this G protein is provided in GenBank accession numbers M27165 and M21557) is employed as the reference VSV G protein.

As used herein, the term “lentivirus vector” refers to retroviral vectors derived from the Lentiviridae family (e.g., human immunodeficiency virus, simian immunodeficiency virus, equine infectious anemia virus, and caprine arthritis-encephalitis virus) that are capable of integrating into non-dividing cells (See, e.g., U.S. Pat. Nos. 5,994,136 and 6,013,516, both of which are incorporated herein by reference).

As used herein, the term “adeno-associated virus (AAV) vector” refers to a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “clonally derived” refers to a cell line that it derived from a single cell.

As used herein, the term “non-clonally derived” refers to a cell line that is derived from more than one cell.

As used herein, the term “passage” refers to the process of diluting a culture of cells that has grown to a particular density or confluency (e.g., 70% or 80% confluent), and then allowing the diluted cells to regrow to the particular density or confluency desired (e.g., by replating the cells or establishing a new roller bottle culture with the cells.

As used herein, the term “stable,” when used in reference to genome, refers to the stable maintenance of the information content of the genome from one generation to the next, or, in the particular case of a cell line, from one passage to the next. Accordingly, a genome is considered to be stable if no gross changes occur in the genome (e.g., a gene is deleted or a chromosomal translocation occurs). The term “stable” does not exclude subtle changes that may occur to the genome such as point mutations.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “host cell” refers to any eukaryotic cell (e.g., mammalian cells, avian cells, amphibian cells, plant cells, fish cells, and insect cells), whether located in vitro or in vivo.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A to 1E show that the approach set forth herein (termed “PRIME” or alternatively “TRACE” for “T7 polymeRAce-driven Continuous Editing”) enabled targeted mutagenesis in mammalian cells within a 2000-bp window with high efficiency. FIG. 1A shows a schematic of the PRIME approach, in which the recombinant protein fusion of cytidine deaminase and T7 RNAP specifically recognizes a T7 promoter upstream of the target gene. The fusion protein subsequently reads through the DNA sequence and introduces site mutations (C·G->T·A). FIG. 1B shows a schematic of constructs designed and used in the instant disclosure. T7 RNAP, T7 RNA polymerase; AID, activation-induced cytidine deaminase; UGI, uracil glycosylase inhibitor; NLS, nuclear localization signal. FIG. 1C shows representative sequencing reads aligned to a subset of the target region in pT7, pAID-T7, and pAID-T7-UGI, respectively. C->T mutations in the aligned reads have been highlighted in green and G->A mutations have been highlighted in red. FIG. 1D shows dot plots of a representative experiment showing C->T (upper panel) and G->A (lower panel) mutation rate per base (%) across the target region (as currently exemplified, a 2000-bp window) in pT7, AID-T7 and pAID-T7-UGI group. Dot plots showing mutation rates in pAPOBEC-T7 and pAPOBEC-T7-UGI are also displayed below, in FIG. 5A.

FIG. 1E shows average C->T (left) and G->A (right) mutation rates of the target region in pAPOBEC-T7, pAPOBEC-T7-UGI, pAID-T7, and pAID-T7-UGI groups (N=3 biological replicates). Background error rate was subtracted (see Example 1: Materials and Methods, below).

FIGS. 2A and 2B show that PRIME enabled continuous somatic mutations in targeted gene loci with high efficiency and negligible off-target effect. FIG. 2A shows that PRIME enabled accumulation of mutations in targeted gene loci over time. EGFP under the control of a T7 promoter was lentivirally integrated into the genome of HEK293T cells. A single integrated clone was transfected with pAID-T7-UGI vs. pAID every 3 days (upper panel). C->T and G->A mutations in the EGFP region were observed to accumulate over a course of 7 days. Lower panel shows results from two biological replicates with the same integrated clone. Background error rate was subtracted. FIG. 2B shows that PRIME exhibited negligible off-target mutation rates in the human genome. Two regions in the human genome with a single-base mismatch from the wild type conserved T7 promoter sequence are highlighted (upper panel). 2000-bp windows (designated as Chr6 & Chr7 locations) immediately downstream of the two T7 promoter-like regions were amplified and sequenced. C->T and G->A mutation rates observed for off-targets (Chr6, Chr7) in pAID-T7-UGI and pT7 group were compared to the on-target mutation rates in pAID-T7-UGI group after 1 week of transfection (lower panel).

FIGS. 3A to 3C demonstrate engineering of the T7 RNA polymerase to achieve high efficiency PRIME. FIG. 3A depicts a schematic showing the mutations in T7 RNA polymerase tested in the Examples of the instant disclosure (upper panel). Bar graphs show the C->T and G->A mutation rates among pEditor variants harboring different mutations in T7 RNA polymerase (lower panel) (N=2 biological replicates). FIG. 3B shows that PRIME-mediated mutation evolved a BFP fluorescence excitation and emission spectra to a GFP fluorescence excitation and emission spectra. In particular, a single H66Y amino acid substitution (CAC->TAC or TAT) caused a shift in the fluorescence excitation and emission spectra of BFP to those of GFP (left panel). Representative fluorescence microscopy images of cells transfected with the indicated editor constructs are also shown (right panel). Scale bar, 100 μm. Scale bar in insets, 15 μm. FIG. 3C summarizes the ratio of GFP-positive cells to BFP-positive cells in each group (N=3 biological replicates).

FIGS. 4A and 4B demonstrate that the PRIME approach maintained the transcriptional activity of T7 RNA polymerase. FIG. 4A shows that fusing a cytidine deaminase to T7 RNAP did not significantly hinder the transcriptional activity of the T7 RNAP. Each pEditor variant was introduced into HEK293T cells together with pTarget in which EGFP gene was solely under the control of a T7 promoter. EGFP signals were observed in cells transfected with pT7, pAPOBEC-T7, pAPOBEC-T7-UGI, pAID-T7, and pAID-T7-UGI, but not in cells transfected with pAPOEBC. Scale bar, 200 μm, which also applies to other micrographs. FIG. 4B shows a schematic of the experimental workflow for calculating the mutation rates of PRIME. Cells transfected with pTarget and pEditor plasmids were incubated for 3 days before being harvested. pTarget plasmids were extracted and PCR reactions were performed to amplify the target region. Sequencing libraries were prepared using the PCR products and next-generation sequencing was performed. Mutation rates in each group, across different pEditor variants, were calculated.

FIGS. 5A to 5C depict that PRIME demonstrated high efficiency and specificity in human cells. FIG. 5A shows dot plots of a representative experiment showing C->T (upper panel) and G->A (lower panel) mutation rates per base (%) across a ˜2-kbp region downstream of a T7 promoter in pT7, APOBEC-T7 and pAPOBEC-T7-UGI groups. FIG. 5B shows that overexpression of cytidine deaminases alone (pAPOBEC or pAID) in the cells resulted in mutation rates that were not statistically different from the background error rates (i.e., the mutation rates in the pT7 group). Each bar is a mean±SD of N=3 biological replicates. FIG. 5C shows bar graphs that display the C->A and G->T (left), C->G and G->C (right) mutation rates observed in pAID-T7 and pAID-T7-UGI groups. Background error rate was subtracted. Each bar is a mean±SD of N=3 biological replicates.

FIG. 6 shows that the PRIME approach demonstrated robust capability in inducing continuous somatic mutations in genomic loci. Plots show observed C->T and G->A mutations in targeted gene loci over a period of 7 days in pAID-T7-UGI vs. pAID group in two additional single cell clones. Background error rate was subtracted.

FIG. 7 displays a table in which features of the instant PRIME approach have been compared with other art-recognized methods for nucleotide diversification.

FIG. 8 displays a reconstruction of cellular lineages produced using the instant TRACE (T7 polymeRAce-driven Continuous Editing) approach over 10 days. Shown are sequence alignments from next generation sequencing (NGS) reads of a cell population that underwent TRACE-mediated diversification. The population was sampled at 4, 7 and 10 days. Highlighted in red and blue are C→T and G→A edits from the consensus. This clonal population was then extracted via consensus editing, and a lineage tree was reconstructed via maximum parsimony.

### DETAILED DESCRIPTION OF THE INVENTION

The current disclosure relates, at least in part, to the identification of a system capable of performing targeted mutagenesis in higher eukaryotic cells, particularly in mammalian cells in culture, across large regions (e.g., 2 kb or more) of targeted nucleic acid sequence, at significantly elevated on-target rates of mutation, as compared to either off-target mutation rates or to background rates of polymerase-mediated mutation. In some aspects, a regions of nucleic acid sequence that is to be targeted for mutagenesis is placed under control of (operably linked to) a bacteriophage promoter (e.g., a T7 promoter), and this promoter-target nucleic acid construct is introduced to a mammalian cell (optionally via transfection). Meanwhile, a nucleic acid construct that encodes for a RNA polymerase (that recognizes the bacteriophage promoter associated with the target nucleic acid sequence) and an operably linked nucleic acid-editing deaminase is constructed and also introduced to the mammalian cell harboring the phage promoter-target nucleic acid construct. The targeted mammalian cell is then cultured for an amount of time sufficient to allow the RNA polymerase to process across the targeted nucleic acid region of interest, and to thereby introduce deaminase-mediated mutants into the targeted nucleic acid sequence during such phage RNA polymerase processing across the targeted nucleic acid.

In certain aspects, the compositions and methods of the instant disclosure therefore provide for enhanced, targeted mutagenesis of mammalian cells, to an extent that is capable of enabling directed evolution of targeted sequences in living cells. As such, application of the instant compositions and methods to drug and/or peptide evolution and screening in mammalian cell lines is expressly contemplated, as are other applications as set forth herein and as are known in the art.

Bacteriophage RNAPs have been previously identified as capable of reading through DNA sequences under the control of a specific promoter without auxiliary transcription factors (8). In particular, the T7 RNAP/T7 promoter system has been previously described as capable of serving as an orthogonal gene expression system in mammalian cells (9, 10). Somatic hypermutation machinery, especially the family of cytidine deaminases, have also been leveraged to induce DNA base switching by catalyzing the deamination of cytosine (C) and subsequent conversion to uracil (U), which is read as thymine (T) by polymerases (11). The instant disclosure has examined whether combining the DNA processivity of bacteriophage DNA-dependent RNA polymerases (RNAPs) with the somatic hypermutation capability of cytidine deaminases could enable continuous, targeted mutagenesis in eukaryotic cells. As demonstrated herein, such a system for pseudo-random integrated mutation of eukaryotic cells (PRIME) is indeed effective and robust.

Various expressly contemplated components of certain compositions and methods of the instant disclosure are considered in additional detail below.

### Bacteriophage Promoters

Certain aspects of the instant disclosure relate to compositions and methods that include bacteriophage promoters, as well as corresponding bacteriophage polymerases, to achieve targeted mutagenesis in mammalian cells across long stretches of sequence. Exemplary bacteriophage promoters of the instant disclosure include, but are not limited to, the following.

T7 Bacteriophage Promoter

The T7 bacteriophage promoter has the sequence 5′-TAATACGACTCACTATAG-3′ (SEQ ID NO: 1). The T7 RNA polymerase initiates transcription at the 3′-terminal guanine (G) of the T7 promoter sequence. The T7 polymerase then transcribes using the opposite strand as a template, processing from 5′->3′. The first base in a T7 polymerase transcript is therefore a guanine (G). The T7 promoter family includes both constitutive promoters and negatively regulated promoters, which can be turned off by a repressor protein. The most common bacterial strain to use with a T7 promoter system is BL21 (DE3) which is an E. coli B strain that contains a λ lysogen with an inducible T7 RNAP gene on the chromosome. However, it is possible to engineer many other E. coli strains to conditionally express T7 RNAP.

T7-Like Bacteriophage Promoters

T7-like bacteriophage promoters most notably include the T3 promoter and the N4 promoter. The T3 promoter has the sequence 5′-AATTAACCCTCACTAAAG-3′ (SEQ ID NO: 2). The bacteriophage T3 and T7 RNA polymerases are closely related, yet are highly specific for their own promoter sequences. T7 promoter variants that contain substitutions of T3-specific base-pairs at one or more positions within the T7 promoter consensus sequence have been previously synthesized and cloned. Template competition assays between variant and consensus promoters have demonstrated that the primary determinants of promoter specificity are located in the region from −10 to −12, and that the base-pair at −11 is of particular importance. Changing this base-pair from G:C, which is normally present in T7 promoters, to C:G, which is found at this position in T3 promoters, was identified to prevent utilization by the T7 RNA polymerase and simultaneously enabled transcription from the variant T7 promoter by the T3 enzyme. Substitution of T7 base-pairs with T3 base-pairs at other positions where the two consensus sequences diverge were also observed to affect the overall efficiency with which the variant promoter was utilized by the T7 RNA polymerase, but these changes were not sufficient to permit recognition by the T3 RNA polymerase. Switching the −11 base-pair in the T3 promoter consensus to the T7 base-pair prevented utilization by the T3 RNA polymerase, but did not allow the T3 variant promoter to be utilized by the T7 RNA polymerase. This probably reflects a greater specificity of the T7 RNA polymerase for base-pairs at other positions where the promoter sequences differ, most notably at −15. Without wishing to be bound by theory, the magnitude of the effects of base substitutions in the T7 promoter on promoter strength (−11C much greater than −10C greater than −12A) were found to correlate with the affinity of the T7 polymerase for the promoter variants, which suggested that the discrimination of the phage RNA polymerases for their promoters was mediated primarily at the level of DNA binding, rather than at the level of initiation (Klement et al. J Mol Biol. 215: 21-9).

N4 Bacteriophage Promoters

N4 bacteriophage promoters comprise conserved sequences and a 3-base loop-5-base pair (bp) stem DNA hairpin structure on single-stranded templates. As an example, N4 Bacteriophage RNAP Polymerase has been identified to bind a 20-nucleotide (nt) N4 P2 promoter deoxyoligonucleotide with high affinity (K_{d}=2 nM) to form a salt-resistant complex. It has also been shown that N4 Bacteriophage RNAP Polymerase interacts specifically with the central base of the hairpin loop (−11G) and a base at the stem (−8G) and that the guanine 6-keto and 7-imino groups at both positions are essential for binding and complex salt resistance. The major determinant (−11G), which has been described as presented to N4 Bacteriophage RNAP Polymerase in the context of a hairpin loop, appears to interact with N4 Bacteriophage RNAP PolymeraseTrp-129. This interaction has been described as reliant upon template single-strandedness at positions −2 and −1. Contacts with the promoter have been described as disrupted when the RNA product becomes 11-12 nt long (see Wigneshweraraj et al. Biomolecules. 5: 647-667, the entire contents of which are incorporated by reference herein, in their entirety).

### Bacteriophage RNA Polymerases

In certain aspects, compositions and methods that rely upon bacteriophage RNA polymerases to achieve targeted mutagenesis in mammalian cells across long stretches of sequence are provided. Bacteriophage-encoded RNA polymerase (RNAP) was first discovered in T7 phage-infected Escherichia coli cells. It was known that phage infection of host bacterial cells led to redirection of host gene expression towards generation of progeny phage particles; however, a previously uncharacterized “switching event” that provoked expression of late bacteriophage genes was first attributed to a phage-encoded RNAP. This phage RNAP was identified as recognizing promoters in the phage genome and expressing phage genes using a single-polypeptide polymerase of −100 kDa molecular weight, which is −4 times smaller than bacterial RNAPs. This was a substantial simplification from the previously known RNAPs from bacteria (5 subunits) and eukaryotes (more than 12 subunits). In spite of its relative simplicity, the single-unit T7 RNAP has been described as able to recognize promoter DNA and unwind double-stranded (ds) DNA to form open complex. After abortive initiation, it proceeds to processive RNA elongation. The simplicity of T7 phage RNAP renders it an attractive model system for study of transcription mechanisms and tool for protein expression in bacterial cells (Basu et al. Nucleic. 30; 237-250). In certain aspects of the instant disclosure, use of the T7 RNAP in concert with nucleic acid-editing deaminases is expressly contemplated for effecting mutagenesis across long stretches of target sequence in eukaryotic cells, particularly mammalian cells. It is also contemplated herein that other polymerases can be used in concert with nucleic acid-editing deaminases, to similar effect. Such other polymerases include, for example and without limitation, T7-like RNA polymerases, such as T3 RNAP, SP6 RNAP and/or N4 RNAP, as described in additional detail below.

T7 RNA Polymerase (T7 RNAP)

T7 RNA Polymerase is an RNA polymerase originally identified in T7 bacteriophage. The T7 RNAP catalyzes formation of RNA from DNA in the 5′→3′ direction. T7 polymerase has been described as extremely promoter-specific and transcribes only DNA downstream of a T7 promoter 5′-TAATACGACTCACTATAG-3′ (SEQ ID NO: 1), with transcription beginning at the 3′ G of the T7 promoter). T7 polymerase has also been described to require a double stranded DNA template and Mg′ ion as cofactor for the synthesis of RNA. It has been described as possessing a very low error rate, and has a molecular weight of 99 kDa (Sousa et al. Progress in Nucleic Acid Research and Molecular Biology. 73: 1-41).

T7-Like RNA Polymerases

T7 RNA Polymerase is a member of a family of single-subunit RNAPs that comprises but is not limited to phage RNAPs including T3 RNA Polymerase, SP6 RNA Polymerase, K11 RNA Polymerase, and N4 RNA Polymerase. These non-T7 RNA polymerases are categorized as T7-like RNA Polymerases.

T3 RNA Polymerase is a member of the DNA-dependent RNA polymerase family and was originally isolated from Bacteriophage T3. It is highly specific to the T3 promoter and transcribes from DNA templates having the T3 promoter. Commercially produced T3 RNA Pol enzyme is expressed from E. coli and is active at 37° C. It has been used in the art for RNA synthesis applications such as for generating in vitro translation templates, hybridization probes, RNA assay substrates, and others.

SP6 RNA Polymerase is a DNA-dependent RNA polymerase isolated from phage-infected Salmonella typhimurium. The enzyme has an extremely high specificity for SP6 promoter sequences (1, 2) and has been described as synthesizing large quantities of RNA from a DNA fragment inserted downstream from a promoter. Strong promoter sequences have been used to construct various cloning vectors, and inserts into the multiple cloning site of these vectors can be transcribed to generate discrete RNAs.

K11 RNA polymerase is an RNA polymerase isolated from gene 1 of the Klebsiella phage K11. It is part of the T7 RNAP family.

N4 RNA Polymerase: Transcription of bacteriophage N4 middle genes is carried out by a phage-coded, heterodimeric RNA polymerase (N4 RNAPII), which belongs to the family of T7-like RNA polymerases. In contrast to phage T7-RNAP, N4 RNAPII displays no activity on double-stranded templates and low activity on single-stranded templates. In vivo, at least one additional N4-coded protein (p17) is required for N4 middle transcription.

### Nucleic Acid-Editing Deaminases

Certain aspects of the instant disclosure relate to compositions and methods that relate to combining the somatic hypermutation capability of a deaminase with the DNA processivity of an orthologous bacteriophage RNA polymerase. Deamination or the removal of an amine group in nucleic acid is carried out by enzymes called deaminases that include, but are not limited to, adenine deaminase, cytidine deaminase (including activation-induced cytidine deaminase), and guanine deaminase.

Adenine deaminases include E. coli TadA, human ADAR2, mouse ADA, and human ADAT2 (see Guadelli et al. Nature. 551: 464-471). Exemplary sequences of adenine deaminases include the following.

tRNA adenosine(34) deaminase [Escherichia
coli str. K-12 substr. MG1655]
(SEQ ID NO: 7):
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD
Escherichia coli str. K-12 substr. MG1655,
complete genome (NC_000913.3) (SEQ ID NO: 8)
TTGTCTGAAGTCGAATTTAGCCACGAATACTGGATGCGTCACGCGCTGAC
GCTGGCGAAACGTGCCTGGGATGAGCGGGAAGTGCCGGTCGGCGCGGTAT
TAGTGCATAACAATCGGGTAATCGGCGAAGGCTGGAACCGCCCGATTGGT
CGCCATGATCCCACCGCACATGCAGAAATCATGGCCCTGCGGCAGGGTGG
TCTGGTGATGCAAAATTATCGTCTGATCGACGCCACGTTGTATGTCACGC
TTGAACCATGTGTAATGTGTGCCGGAGCGATGATCCACAGTCGCATTGGT
CGCGTGGTCTTTGGTGCGCGTGACGCGAAAACTGGCGCTGCGGGATCTTT
AATGGATGTGCTGCATCATCCGGGTATGAATCACCGAGTGGAAATTACGG
AAGGAATACTGGCGGATGAGTGCGCGGCGTTGCTCAGTGACTTCTTTCGC
ATGCGCCGCCAGGAAATTAAAGCGCAGAAAAAAGCGCAATCCTCGACGGA
TTAA
Homo sapiens adenosine deaminase RNA specific
B1 (ADARB1, also known as ADAR2), transcript
variant 1, mRNA (NM_001112.4; SEQ ID NO: 9)
GAGGCGCTGAGGCGGCCGTGGCGGCGGCGGCGGCGGCGGCGGCAGCGGCG
GCCAAGCGGCCAGGTTGGCGGCCGGGGCTCCGGGCCGCGCGAGGCCACGG
CCACGCCGCGCCGCTGCGCACAACCAACGAGGCAGAGCGCCGCCCGGCGC
GAGACTGCGGCCGAAGCGTGGGGCGCGCGTGCGGAGGACCAGGCGCGGCG
CGGCTGCGGCTGAGAGTGGAGCCTTTCAGGCTGGCATGGAGAGCTTAAGG
GGCAACTGAAGGAGACACACTGGCCAAGCGCGGAGTTCTGCTTACTTCAG
TCCTGCTGAGATACTCTCTCAGTCCGCTCGCACCGAAGGAAGCTGCCTTG
GGATCAGAGCAGACATAAAGCTAGAAAAATTTCAAGACAGAAACAGTCTC
CGCCAGTCAAGAAACCCTCAAAAGTATTTTGCCATGGATATAGAAGATGA
AGAAAACATGAGTTCCAGCAGCACTGATGTGAAGGAAAACCGCAATCTGG
ACAACGTGTCCCCCAAGGATGGCAGCACACCTGGGCCTGGCGAGGGCTCT
CAGCTCTCCAATGGGGGTGGTGGTGGCCCCGGCAGAAAGCGGCCCCTGGA
GGAGGGCAGCAATGGCCACTCCAAGTACCGCCTGAAGAAAAGGAGGAAAA
CACCAGGGCCCGTCCTCCCCAAGAACGCCCTGATGCAGCTGAATGAGATC
AAGCCTGGTTTGCAGTACACACTCCTGTCCCAGACTGGGCCCGTGCACGC
GCCTTTGTTTGTCATGTCTGTGGAGGTGAATGGCCAGGTTTTTGAGGGCT
CTGGTCCCACAAAGAAAAAGGCAAAACTCCATGCTGCTGAGAAGGCCTTG
AGGTCTTTCGTTCAGTTTCCTAATGCCTCTGAGGCCCACCTGGCCATGGG
GAGGACCCTGTCTGTCAACACGGACTTCACATCTGACCAGGCCGACTTCC
CTGACACGCTCTTCAATGGTTTTGAAACTCCTGACAAGGCGGAGCCTCCC
TTTTACGTGGGCTCCAATGGGGATGACTCCTTCAGTTCCAGCGGGGACCT
CAGCTTGTCTGCTTCCCCGGTGCCTGCCAGCCTAGCCCAGCCTCCTCTCC
CTGTCTTACCACCATTCCCACCCCCGAGTGGGAAGAATCCCGTGATGATC
TTGAACGAACTGCGCCCAGGACTCAAGTATGACTTCCTCTCCGAGAGCGG
GGAGAGCCATGCCAAGAGCTTCGTCATGTCTGTGGTCGTGGATGGTCAGT
TCTTTGAAGGCTCGGGGAGAAACAAGAAGCTTGCCAAGGCCCGGGCTGCG
CAGTCTGCCCTGGCCGCCATTTTTAACTTGCACTTGGATCAGACGCCATC
TCGCCAGCCTATTCCCAGTGAGGGTCTTCAGCTGCATTTACCGCAGGTTT
TAGCTGACGCTGTCTCACGCCTGGTCCTGGGTAAGTTTGGTGACCTGACC
GACAACTTCTCCTCCCCTCACGCTCGCAGAAAAGTGCTGGCTGGAGTCGT
CATGACAACAGGCACAGATGTTAAAGATGCCAAGGTGATAAGTGTTTCTA
CAGGAACAAAATGTATTAATGGTGAATACATGAGTGATCGTGGCCTTGCA
TTAAATGACTGCCATGCAGAAATAATATCTCGGAGATCCTTGCTCAGATT
TCTTTATACACAACTTGAGCTTTACTTAAATAACAAAGATGATCAAAAAA
GATCCATCTTTCAGAAATCAGAGCGAGGGGGGTTTAGGCTGAAGGAGAAT
GTCCAGTTTCATCTGTACATCAGCACCTCTCCCTGTGGAGATGCCAGAAT
CTTCTCACCACATGAGCCAATCCTGGAAGAACCAGCAGATAGACACCCAA
ATCGTAAAGCAAGAGGACAGCTACGGACCAAAATAGAGTCTGGTGAGGGG
ACGATTCCAGTGCGCTCCAATGCGAGCATCCAAACGTGGGACGGGGTGCT
GCAAGGGGAGCGGCTGCTCACCATGTCCTGCAGTGACAAGATTGCACGCT
GGAACGTGGTGGGCATCCAGGGATCCCTGCTCAGCATTTTCGTGGAGCCC
ATTTACTTCTCGAGCATCATCCTGGGCAGCCTTTACCACGGGGACCACCT
TTCCAGGGCCATGTACCAGCGGATCTCCAACATAGAGGACCTGCCACCTC
TCTACACCCTCAACAAGCCTTTGCTCAGTGGCATCAGCAATGCAGAAGCA
CGGCAGCCAGGGAAGGCCCCCAACTTCAGTGTCAACTGGACGGTAGGCGA
CTCCGCTATTGAGGTCATCAACGCCACGACTGGGAAGGATGAGCTGGGCC
GCGCGTCCCGCCTGTGTAAGCACGCGTTGTACTGTCGCTGGATGCGTGTG
CACGGCAAGGTTCCCTCCCACTTACTACGCTCCAAGATTACCAAGCCCAA
CGTGTACCATGAGTCCAAGCTGGCGGCAAAGGAGTACCAGGCCGCCAAGG
CGCGTCTGTTCACAGCCTTCATCAAGGCGGGGCTGGGGGCCTGGGTGGAG
AAGCCCACCGAGCAGGACCAGTTCTCACTCACGCCCTGACCCGGGCAGAC
ATGATGGGGGGTGCAGGGGGCTGTGGGCATCCAGCGTCATCCTCCAGAAC
CTCACATCTGAACTGGGGGCAGGTGCATACCTTGGGGAGGGAGTAGGGGG
ACACGGGGGACCACCAGGTGTCCACGGTTGTCCCCAGCATCTCACATCAG
ACCTGGGGCAGGTGCGCAGTGTGGGGAGGGGATGGGGTGCGTCAGGGCCC
AGCATCGCCGCCTGGCATCTCTCTGCCGCAGCATTTCCCCTTCTGAACCG
TCCAGTGACTGCTTTCAATCTCGGTTTACGTTTAGAAATTGAGTTCTACT
GAGTAGGGCTTCCTTAAGTTTAGGAAAATAGAAATTACTTTGTGTGAAAT
TCTTGAATAAATAATTTATTCAGAGCTAGGAATGTGGTTTATAAAATAGG
AAGTAATTGTGTCAGGTCACTTTTATGCCACATTATTTTAATTGCAAAAA
AGCATCTATATATGGAGGAGGGTGGGAAAATAGAGGTAGGAAATAGTAGC
CTAAAGGAAATCGCCACACGTCTGTCTAAACTTAGGTCTCTTTTCTCCGT
AGGTACCTCCCTGGGTAGTTCCACACACTAGGTTGTAACAGTCTCTCCCT
GAGGAGCAGACTCCCAGCATGGTGTAGCGTGGCCCTGTCATGCACATGGG
GTCCCGCAGCAGTGACTGTGTGTCCTGCAGAGGCGTGACCCAGGCCCCTG
TAGCCCTCAGCCTCCTCTAGAAGCTTCTGTACTCCTTGTAGGATCAGATC
ATGGAAAACTTTTCTCAGTTTACTTCTAAGTAATCACAGATAATACATGG
CCAGTAATCCCAGGCTGGCCATTCATTCAGGTTTTTTAAAGGATATTTAA
CTTTTATGGACTAGAAGGAATCACGAGGGCTACTGCACAATACATGGCCT
AAGTTCCCTCTGTTCCTTCCTCTGAATCGAATGGATGTGGGTGACCGCCC
GAAGGCCTTCACAGGATGGAAGTAGAATGATTTCAGTAGATACTCATTCT
TGGAAAATGCCATAGTTTTAAATTATTGTTTCCAGCTTTATCAAAGACAT
GTTTGAAAAATAAAAAGCATCCAAGTGAGAGCTGGTGAGACCACGTGCTG
CTGGCGTAGTGTAGGCCAGACATTGACAGTCCTGACGGGAGCTCAGGGCT
GCCCAGCGCCCAGCGTGCACGGGACGGCCCCACGACAGAGGGAGTCAGCC
CGGGAGGTCAGGAGCGCGGCGGGCGAGGGCCCTGTGTGGACCACCTCCAC
CAAGCTCAGAGATTTGCACCAGGTGCCTTGTTGCCTCCGCTCAGGATGAA
AGAGGAGCTGAGAGAAGTGCTCTGCCTGCCAGTGCAGTGCCCAGCTCCAA
GGCTCTAGAGGGTGTTCAGGTGGGTCTCCTGGGGCCATGGGGAGAGATTG
GTGCAGACCTTACCCCACAGCATACACCTGCCACAGCGAAATCCAGGGTG
TTGGCACCTGTGTGTCCGTGATGAGCCTAGGAAACCAGAGCAGGGGCAGA
GGGGCGTCATCCTCCCACCGGACGCTGGGAGCTCAGACCCCAAAACTGAA
ACACCGTGGCTTCGGCGGGGGGTGTGCCTCCTGATGTCAGGAGCCCCATC
CACGTGTGTCCACACAGATCTCGTCGCAGCACGGCAGGAAGGGGTGCTGC
TTAGGGCTCATTGTTGGGGACATGACCGGGTTCAGCGGCTAGAACATCTG
CCCCACAGCAGCCTCCTCCTCCACCGAAGAGGGTAGTTGTCTCCCTGAAG
CAGTCACAGCAGGCGTCTCTGCCGCTCCGTCACCACAGTGGGGTTTTGTT
CAGGCAGATCGCGCTGGGGTTCTGCACCTGCAGAAGGAGAGGGGTCTGTT
GTCGCTGGCTTTCCCCCAAGCAGGCTCTTGCACACTCTAGAAAAAACACC
TTGTAAGTCTGTGCATTTTTATTGTCTTGATAAATTGTATTTTTTTCTAA
TGGGGATTGGGAGATGGACTTCGTTTTTAAAAATATGTGGATTTTGGTTA
CCAAGTTTAGTGTTAATATATTCCATATACATACAAAACTACCCGGTATG
TCTGGCTTTTCCCTTCTGTCAGGTAATAGCTAAAGTCAGCATGATTGCTC
CCTGTACCACCCCAAATAAGTGAGTGCCTCACCTTGTGGGGCCTGAGCAG
CTACCTTGAGACCATGTGAGGTGGCACCTTTCCGGGGTGGACTCGTGCGG
CCTTGAGGACAGGCACAGGGCACCCTATCCCAAGCCGTCCAGGCAGGAGG
AAGGCAGCCAAGGCAACTGGGTTCTGGGAGCCCTGGGTGGGGCAGCTGTG
GGGAGGAACTGGGTTCGGGGAGCCCTGGGCGGGGCGGCTGTTGGGGGGAA
CTGGGTTCGGGGTGCCCTGGGCAGGGGGCTACTGGGGGGCGGCTGTGAGG
AGGAGTTGGGTTCAGGGAGCCCTGGGCGGGGTGGCTGTCAGGGGGAACTG
GGTTCCGGGAGCCCTGGGCCGGGGCAGGGGGCGGCTGTAGGAAGGAACTG
GTTTCGGGGAGCCCTGGGCGGGGCGGCTGTGGGGAGGAAGGTGACGTGCA
GGGGACCAGAGGCTCTGCACTGCTCCTAGGACAGCTCATCTGTAATCAGA
AAAAAAATAAACAAAATACAGAACGCTGACTCCTCCGTGAGACAGATCGG
GGACCTTAGCACTTTAATCCCTCCCTTCTGAGCGCTCGGTGTGCACTTTT
AGACTATAGCTGTTTCATTGACGTGTCACTCTCCATCCAGTGTCCTTGAT
GTGGCTTTTAGAGACTTAGCAGAAAATTCGACACAAGCAGGAACTTGATT
TTTTAAGAAAAAATATTACATTTTGAGGACATTTTGACAAGTAGGGGAAG
AGAGGGCTTCTGTTGTTTTGTTTTGTTTTGTTTTGTTAACTAAACCTGAA
GTATTAATTCCACAAAGACACTGTCCCTCAGGACCACTCAGGTACAGCTC
TGCCAGGGACAGAGTCCTGCTAGTGGGAGGTCTCAGGTGGGGCGGTGTGT
TCTGTGCCATGAGGCAGCGACAGGTCCAGATGGATGTCGTCACCACCTTC
CTCAGCTCTCATCACCTGGTCGTACGCCAGGCCCACCTCTTCCCAGCAAG
GGACGCCAAAGAACTGCAGTTTTTATTCTGAGTCTTAATTTAACTTTTCA
TCATCTTTTCCTATTTTGGAGAATTTTTTGTAATTAAAAGCAATTATTTT
AAAATGTGCAAGCCAGTATCTCACAAGGCATGGATTTCTGTGGAATTTAT
TTTTATTCAAATAACCATATTTATCTCCAGGCTGTGGAATCGCCACTTTC
TTTGTGAAGACAGTGTCTCTCCTTGTAATCTCACACAGGTACACTGAGGA
GGGGACGGCTCCGTCTTCACATTGTGCACAGATCTGAGGATGGGATTAGC
GAAGCTGTGGAGACTGCACATCCGGACCTGCCCATGTCTCAAAACAAACA
CATGTACAGTGGCTCTTTTTCCTTCTCAAACACTTTACCCCAGAAGCAGG
TGGTCTGCCCCAGGCATAAAGAAGGAAAATTGGCCATCTTTCCCACCTCT
AAATTCTGTAAAATTATAGACTTGCTCAAAAGATTCCTTTTTATCATCCC
CACGCTGTGTAAGTGGAAAGGGCATTGTGTTCCGTGTGTGTCCAGTTTAC
AGCGTCTCTGCCCCCTAGCGTGTTTTGTGACAATCTCCCTGGGTGAGGAG
TGGGTGCACCCAGCCCCGAGGCCAGTGGTTGCTCGGGGCCTTCCGTGTGA
GTTCTAGTGTTCACTTGATGCCGGGGAATAGAATTAGAGAAAACTCTGAC
CTGCCGGGTTCCAGGGACTGGTGGAGGTGGATGGCAGGTCCGACTCGACC
ATGACTTAGTTGTAAGGGTGTGTCGGCTTTTTCAGTCTCATGTGAAAATC
CTCCTGTCTCTGGCAGCACTGTCTGCACTTTCTTGTTTACTGTTTGAAGG
GACGAGTACCAAGCCACAAGAACACTTCTTTTGGCCACAGCATAAGCTGA
TGGTATGTAAGGAACCGATGGGCCATTAAACATGAACTGAACGGTTAAAA
GCACAGTCTATGGAACGCTAATGGAGTCAGCCCCTAAAGCTGTTTGCTTT
TTCAGGCTTTGGATTACATGCTTTTAATTTGATTTTAGAATCTGGACACT
TTCTATGAATGTAATTCGGCTGAGAAACATGTTGCTGAGATGCAATCCTC
AGTGTTCTCTGTATGTAAATCTGTGTATACACCACACGTTACAACTGCAT
GAGCTTCCTCTCGCACAAGACCAGCTGGAACTGAGCATGAGACGCTGTCA
AATACAGACAAAGGATTTGAGATGTTCTCAATAAAAAGAAAATGTTTCAC
TA
Homo sapiens adenosine deaminase RNA specific
B1 (ADARB1, also known as ADAR2) protein
(NP_001103.1; SEQ ID NO: 10))
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQ
TGPVHAPLFVMSVEVNGQVFEGSGPTKKKAKLHAAEKALRSFVQFPNASE
AHLAMGRTLSVNTDFTSDQADFPDTLFNGFETPDKAEPPFYVGSNGDDSF
SSSGDLSLSASPVPASLAQPPLPVLPPFPPPSGKNPVMILNELRPGLKYD
FLSESGESHAKSFVMSVVVDGQFFEGSGRNKKLAKARAAQSALAAIFNLH
LDQTPSRQPIPSEGLQLHLPQVLADAVSRLVLGKFGDLTDNFSSPHARRK
VLAGVVMTTGTDVKDAKVISVSTGTKCINGEYMSDRGLALNDCHAEIISR
RSLLRFLYTQLELYLNNKDDQKRSIFQKSERGGFRLKENVQFHLYISTSP
CGDARIFSPHEPILEEPADRHPNRKARGQLRTKIESGEGTIPVRSNASIQ
TWDGVLQGERLLTMSCSDKIARWNVVGIQGSLLSIFVEPIYFSSIILGSL
YHGDHLSRAMYQRISNIEDLPPLYTLNKPLLSGISNAEARQPGKAPNFSV
NWTVGDSAIEVINATTGKDELGRASRLCKHALYCRWMRVHGKVPSHLLRS
KITKPNVYHESKLAAKEYQAAKARLFTAFIKAGLGAWVEKPTEQDQFSLT
P
Mus musculus adenosine deaminase (Ada),
transcript variant 1, mRNA (NM_001272052.1;
SEQ ID NO: 11)
AGCGTGGGCGGGGCTGTGCCGGGGCAGCCCGGTAAAAAAGAGCGTGGCGG
GCCGCGGTCTCTGAGAGCCATCGGGAAGCGACCCTGCCAGCGAGCCAACG
CAGACCCAGAGAGCTTCGGCGGAGAGAACCGGGAACACGCTCGGAACCAT
GGCCCAGACACCCGCATTCAACAAACCCAAAGTAGAGTTACACGTCCACC
TGGATGGAGCCATCAAGCCAGAAACCATCTTATACTTTGGCAAGAAGAGA
GGCATCGCCCTCCCGGCAGATACAGTGGAGGAGCTGCGCAACATTATCGG
CATGGACAAGCCCCTCTCGCTCCCAGGCTTCCTGGCCAAGTTTGACTACT
ACATGCCTGTGATTGCGGGCTGCAGAGAGGCCATCAAGAGGATCGCCTAC
GAGTTTGTGGAGATGAAGGCAAAGGAGGGCGTGGTCTATGTGGAAGTGCG
CTATAGCCCACACCTGCTGGCCAATTCCAAGGTGGACCCAATGCCCTGGA
ACCAGACTGAAGGGGACGTCACCCCTGATGACGTTGTGGATCTTGTGAAC
CAGGGCCTGCAGGAGGGAGAGCAAGCATTTGGCATCAAGGTCCGGTCCAT
TCTGTGCTGCATGCGCCACCAGCCCAGCTGGTCCCTTGAGGTGTTGGAGC
TGTGTAAGAAGTACAATCAGAAGACCGTGGTGGCTATGGACTTGGCTGGG
GATGAGACCATTGAAGGAAGTAGCCTCTTCCCAGGCCACGTGGAAGCCTA
TGAGGGCGCAGTAAAGAATGGCATTCATCGGACCGTCCACGCTGGCGAGG
TGGGCTCTCCTGAGGTTGTGCGTGAGGCTGTGGACATCCTCAAGACAGAG
AGGGTGGGACATGGTTATCACACCATCGAGGATGAAGCTCTCTACAACAG
ACTACTGAAAGAAAACATGCACTTTGAGGTCTGCCCCTGGTCCAGCTACC
TCACAGGCGCCTGGGATCCCAAAACGACGCATGCGGTTGTTCGCTTCAAG
AATGATAAGGCCAACTACTCACTCAACACAGACGACCCCCTCATCTTCAA
GTCCACCCTAGACACTGACTACCAGATGACCAAGAAAGACATGGGCTTCA
CTGAGGAGGAGTTCAAGCGACTGAACATCAACGCAGCGAAGTCAAGCTTC
CTCCCAGAGGAAGAGAAGAAGGAACTTCTGGAACGGCTCTACAGAGAATA
CCAATAGCCACCACAGACTGACGCAGGGCGGGTCCCCTGAAGATGGCAAG
GCCACTTCTCTGAGCCTCATCCTGTGGATAAAGTCTTTACAACTCTGACA
TATTGACCTTCATTCCTTCCAGACCTTGGAGAGGCCAGGTCTGTCCTCTG
ATTGGATATCCTGGCTAGGTCCCAGGGGACTTGACAATCATGCACATGAA
TTGAAAACCTTCCTTCTAAAGCTAAAATTATGGTGTTCAATAAAGCAGCT
GGTGACTGGTATCTTGCAGCACATGGTGAATATGGTCTCGGGGCTGCTGG
CTAGGATGCTAAGAAAGGAGGAGCCCTGGGCCCTACGCTGAGTGTCAGGC
TGGGGAGCCAGGGTCTCTTTCCTGCAGAAGCGATTCTTTCCCAGAGGGGC
TGTTGGAGCAGATGCTCCTGAACTCTCCGCCCCTTTAACCAGTCCTTTGG
ATTTATTTTTATTATTTTTAAATATTTAATTATGTTTATGTATATGGGTG
TTTT
Homo sapiens adenosine deaminase tRNA
specific 2 (ADAT2), transcript variant 1,
mRNA (NM_182503.3; SEQ ID NO: 12)
CTCTGCCGCGGGCTCTGTAGCTGAGTGGTGGCTGGGTATGGAGGCGAAGG
CGGCACCCAAGCCAGCTGCAAGCGGCGCGTGCTCGGTGTCGGCAGAGGAG
ACCGAAAAGTGGATGGAGGAGGCGATGCACATGGCCAAAGAAGCCCTCGA
AAATACTGAAGTTCCTGTTGGCTGTCTTATGGTCTACAACAATGAAGTTG
TAGGGAAGGGGAGAAATGAAGTTAACCAAACCAAAAATGCTACTCGACAT
GCAGAAATGGTGGCCATCGATCAGGTCCTCGATTGGTGTCGTCAAAGTGG
CAAGAGTCCCTCTGAAGTATTTGAACACACTGTGTTGTATGTCACTGTGG
AGCCGTGCATTATGTGTGCAGCTGCTCTCCGCCTGATGAAAATCCCGCTG
GTTGTATATGGCTGTCAGAATGAACGATTTGGTGGTTGTGGCTCTGTTCT
AAATATTGCCTCTGCTGACCTACCAAACACTGGGAGACCATTTCAGTGTA
TCCCTGGATATCGGGCTGAGGAAGCAGTGGAAATGTTAAAGACCTTCTAC
AAACAAGAAAATCCAAATGCACCAAAATCGAAAGTTCGGAAAAAGGAATG
TCAGAAATCTTGAACATGTTCTGATGAAAGAACCAAGTGACCCAAAGTGA
CCTGGACAAGATTCATAGACTGAAAGCTGTTGACATCGTTGAATCATATG
TTTATATATTGTTTTTAATCTGCAGGAAAATGGTGTCTCTCATCATTTGC
TCTGTTAAGGGAACAAATTAGCACTTTTTAGAAGTCTGACAATTGTAAAC
AGTTATTAGCTTTTCCAGAAGCTGATTCCCATTTTAAGATGGGGGAAAAT
TAAGGTTTGAGGTTTTAGAAATTAGCAAGTAGTGCATACCCTTCTAGCCA
CAAGTGCCCAGTCCAGGCAAGTGCTGACTTCTTAGAGAATGTGTGGCCAG
ACCCAGGGACCTGGAGTGTGTTTGGACTGCAGTTTGCCACCCTGAGAACA
CCTTCTCCAGGACTGGCATTTCAGAATCAGATTCTTCATTTTTTGCAGCT
ACGATGTTCTTCCAGGGCACTGGGGGCTGTGACTTCTCTCTAAATTGTAT
ATAAGTTGTGTATATAGAGACCATAATTATATGGTCCTTAGAAAAGACTT
TGCTTTTATAAAGCATTTAGAAAAAATGCATACTTTTAAAACAAGTGCTT
GAGTTGTCACTTAAAAATTATAGCATATTGCTATAATAAAACCTTATTTA
TGTCTTATTTGAAGATGAATAGTCTTAAAAGATAAAGACATAAATGGGAC
AATTGTTATTGAGCAAAAAACCAAATTATCCCACCCTCATGGAGCTTATA
TTCTAGCAAGGGGAGATGGATATGATAGATTACACAGTTTATTGGAGGAC
AATAAGAGTTATGGCAAAAAGCAAAAGGAACACAGGGTAAAGGGGATAGG
TGCCATTTGGTGGTGAGAATGCTGACTGAAAAATAGAATGATCAATTTAA
TCTGAAACAAATGGTTATTTCTTTTATAATCCATATAATAAATTTAAAAT
CTAAAATGTAAAATTTTGAACACAACACTGGAAAGGGTATCCACAGCAGG
AAGTCCCCAGTTCACCTCCATGACTACAGGGCAGCTTTGCACAGCCCTCT
GGGCGCACTGTGTGCCTCTGCCCAGAAGGGGGCCTCGCCGTTCCACCAGA
AGCTCAGCTCCAGGCCCTGGAGGGGCTGCTGCTCCTCAGTTGCATTTCTT
CAGTAGATTCATTTCCTTGATGCAAAGCATCTGTATTTGTTGGTTCTGTC
ATTTGAGCGATGTCTCTGACTTGTTTGTTTTGAATTACATTACAGGCTGG
AATGTAATTGTGGTGAAAGTATTTTTATATTGCTGAGAGTAGCAGCTAAT
CACAGTTACATGCTTCAGAGGACTTATAATTGCTTGGTTTTGTGTGTGTG
TGTGTGTGTGTGTGTGTGTGTGTGTGTGTTTAACTGCATTTGAAAAGTTT
TATGGAGAATATGCATGATTTTAAATCTGTGATAATGTTACATGCACCTT
CAATTTCATCCACTTTAAAAATTATCTTCTCATTGAATTTTAGTGCTTCT
ACTAGTTTGTTCCTTTTTGCAGTTGGTCGTAATTCATTTCTGGCTTCTTA
TGCTTTCCTGCAAGCAGATTTCATTGCATTTATTGTGTTCATATCATTTT
CTTGGGGATTATTTGTAGGACAACCAACCTGGAGTTTTGCCTCTCTAGAG
TACCACCCAGTAAGTCTGGCTGAGCATCTTATGTCCAGTAGGTTCTTGGT
AAACATTTGCTAAATGAAATTACTGATTGAAATTTGGGGAAAAGTGAATA
AGAAGACTATCTAGGACAAAAAGCCAAAGCCGAAAATAGTATATGAGCAT
TCTAGCCCAGAGACTGTCGCTACTAAAAGAATGAAGGAAATAATAAAGTG
ATAGACAGGGAAGGATAGAAAAGACTTAACAATATACATATGTTCCGTCT
TTGCTGTTTTGGAGAATGATGGATAAGTAGTGTTTCCTGATTCTGAAGCA
TAGCTGAACAATTTAATTGTGGTTTACCATCTTTTTGGTTCCCTCTTCAG
TAATTAACCTATCGAAAATCTGTCCTAAATGTTTGGACTGGGGCACAGTT
CCCTCCATCGCTTTGGGAGAAAATCATTAATATGGCATACTGCAGATTGG
AGGGCAGGACCACTGAGGGTGTCATAGACATTAGCTCTATGGAATTCTGC
TAGCAATTTCCAAGTGACAGTGAGGAATTATGGATATATGTTGAGGTCAT
TCAGCTTCCTGAGTACCACATTCCCCAGCTACTTAGACACGGGTTAAAAT
ATTAAGATGTCCTAGTTCAACAGCTTGAATTCCATTGATTGATACTGATA
GTGCCTGTCCAAGACACCAGCTGAAAGACTTGTTTTGTGTACAAAATAGT
TCTGAAAGTGGTGAGATACAAAAAGGTTTTAGAATCACTGCCCTGTTGAG
AGAAATTAGGGGGAAATGATTACATTTAGAAGCTGCTAGAGTTATCCAGT
GTTTGCTGGTCTTTGCAACAAACTGTGGAGAATGGGTGGTATGTAATGCT
TTGGTAGGCTTCAATCACTGATAAAAGATCATGTTAAAATATCTTTGTGC
TTTCTTGTTACTTGGCACAACCATCTCTTCCTGTGTTGTATTTGGAGTAT
CATGGAGAGAAAATAGATGGCCAAGAGCTTCAGTGTAGGCAAGAACTCTT
AATTTTTCTTTAAACTTTTTACTGGGAAAAGTATATATATATAAAATACA
CACACACACACACACACACACACACACACACACACACACACAAACACAAC
ACACCATGGCCCTTTACCCCGAAATGCTTCAGTATAGTTATTGACTTAAG
TAAATTTAACATTGATATACTTGAATCTATCATTTGTATTACAGTTTTGT
CAGCTGACCCAATAATGTCCTGTAAAGAAGTTCTCCCACTACCCTATAAT
CCCAGGTCCAGTCTAGGGTCCAGCATTACATTTACTTGTCTTGAATCCAG
CTTTTTCTTTTTTTTTTTTTTTTTTGAGATAGGTCTCACTCTGTCGTCCA
GTGGCATGATCACAGCTCACTGCAGCCTCAACCTGGCTCAAGCAATCCTT
CCTCCTCAGCCTCCTGAGTAGCTGGGACCACAGACTCATGTCACCACACC
TAATTTTTTTTTTTTTTTTTTTTTGTAGAGACAAGGTCTCACTATGTTGC
CCAGGCTGGTCTTGAACTCCTAGGCTGAAGCAATCCTCCTTCCTTGGCCT
CCCAAAGCACTGGGATTATAGACGTGAGCCACTGCACCGGTCTGCCTTTA
GCTTCTTTTAGTCTAGAACATTTTCACTGGCTTTCTTTGTCTTTTATGAC
ATTGACATTTTTAAATAATACAGTCATTTTGCCTCCTTTCTGTTTTCTTC
TTCTTTTTTTAAATAATAGAATGGTCCTTGTTTTAAATTTATTTGATATT
TTCTTGTGATTAGATTCAGGTGCTGGTTGATGTTAAGTTCCTCACAGGAT
ATCACATCTGGAGGCACACAAAGGCCGTCACACCAAGGTGATGTCAATTT
TGGTCATCTGGTCAAGGTGTTGTCCTATTCCTTCACTATATAGTTACCTT
TTTTCTCTGTTGCAATGAATAAGCAGTCTGTGGGAAGAGGAGCTGTTACA
TTTTAAACAGAAAATGTATTTGACACTGATGGAAAGGAGAGGAGGAAAAT
TAATGACATAAATTTCAAAGCAACTATTAAATTATTTGATTGCATTCTTC
CTCTTTTACTGTCTGCCAAAATTGATAAAAAAAATTTTTCTAATAAGAAT
GTTTTAAATAGTGATATCTTAATAAGCATCAAAATTAAGCCTGAGAAATA
AATTCTTTCCTTCCTAATTTCCTCCTCAGCAAAAGTAATAATTATATAAA
TTTCATTATGCCTGATAAGATAGGGTTTTGGAAAATAGACCTAAGATGTT
TCTGATACTGCAGATGACCTATGGTGATCCAATGGGATAAACACTCTAGG
TAGGTTGTCATTTGGTCATAAAATATGAGTTATCTTGGGTTTCCATAGAG
ACATCTAGACTTAAAATGTTGTAAGCACTGCTACTTTCAAAATGTCAGTA
AAAATAGCAAAAGCCAAAGCTCTTGAAAAAATTACTTAAATCTTTTTTAA
AAGTAGTATAGCGCCTTGTTAAAAATCTGTGGTGATGCCAAAGCTTGTCT
TTCCCAGTGGTCCTACGTGAACTGGCCTTATAGCCCCAGGGAAACCAGAC
ACCAGGAATTGGTTTCTCTGCCTTTTGGCAAAGGAATAAGACTACATTGA
CTTCATCTATGAAGACAACTGCCAACTATTTCCTTTGTAAATTGCTAATT
TTGTGTAGTGAGGAAAGGAGCGATGGGCGACGTGATTTTTATGGATTAGA
CTGGTGAGTTCTGCTGAAAGTTTGACATCTTTAGGATCTTACATTTTCTT
CAAGTTGAGCTAATGAAAACAGGCTCGTGACTATTTATCACCTGATTTCT
AAGTGGATATTGGGTTGAACACCACATATCCATGACTATTAAGGAGGCTT
CATGGTGTAGTTTGACAAAGGCTCTCTCCTTGACCAAACTTCAGTCAGGC
CCTAAGTCCTCTTTTTAACCAGGCCTCCACCTTGGCCCCCATTCTTGATG
GGCCTATACAGCCCAGCTTTAGCAAGAATCCTGCTAAGCTAGTTTAGAGA
GAATCCCACATCCCCAATATCTATGAAATTTCTCATCCCCTACTTTTGAT
GTGTAAGTCCTTGGCCTCCCTTCAACGAGAAGCCTGTTAAGTTCATTTTG
CAAGAACTCTACTCTTGATATCTCCTCTTAGTAATTTCCTAATCACTGAC
CCCCTCACTCTGCCCATTAGTTATAAACCCCCACATGTTCTGGTTGTATT
CAGAGCTGAGCCTGATCTCTTCCTCTTGTTGGGATAGTTTTAAAACCTGC
GATAGTTTTAAAACCTATCACTGTAGTCCTGAATTAAGTCTTCCTTACCT
TAACAAGTGTCAAAATAAATTTTTCTTTAACATGTTGAAGCATGAACTTG
AGAATCTAGAGCAGGAGTCCACAAAGTATGGCCCATGGGCCATATCCAGC
CCGCTGCCGGTTTCGGTACCACTCATGACTTAAAAATGGGTCTTACAATT
CTGAGTGATTGAAAAAAAATCAAAAGAAGGATAATATTTAGTGACCCATG
AACCTTATATGGCAATCAAATTTCAGTGTCCATAAATAAAGTTACATTGG
ATGACAGCCATGCCCATTTGTTTCTGTGTTGTCTGTGGCTGCTCGTGTGC
TACAATGGCAGAGTTGAGCAGTGGTGACAAACCATGCGACTCACAAAGGC
CTAAAATATTTAGCGTCTGGCCCTTCGAGAAAATGTTAGCTGCCCCTGGT
CTAGAGTAGGTAAAAGGCTGAGATTGGAAGCTGCTTGTTCAAATTCTGTG
ATTGGAACCGAATGATGTGGCTCATTGTACAGCTCATGGTGAATTGCTTC
AGTACCATGGTTTTGTTTTTTCCTTTTGAAAAGTTGGTCTATAAATGTAA
AGGAAAAATCTAAGATACCAAAATATGTTTTCTGGCTTAGAATGTTTTAT
TTCCTTGTATACATTTTAAGAGAGTGGCAAGGAGAAAAGATAATGTATCA
TTTTATTTGGGTTTAGAATAAATAATACATTTTATTTATGATCA
Homo sapiens adenosine deaminase tRNA
specific 2 (ADAT2), transcript
variant 1, protein (NP_872309.2;
SEQ ID NO: 13)
MEAKAAPKPAASGACSVSAEETEKWMEEAMHMAKEALENTEVPVGCLMVY
NNEVVGKGRNEVNQTKNATRHAEMVAIDQVLDWCRQSGKSPSEVFEHTVL
YVTVEPCIMCAAALRLMKIPLVVYGCQNERFGGCGSVLNIASADLPNTGR
PFQCIPGYRAEEAVEMLKTFYKQENPNAPKSKVRKKECQKS
Mus musculus adenosine deaminase
(NP_001258981.1; SEQ ID NO: 14)
MAQTPAFNKPKVELHVHLDGAIKPETILYFGKKRGIALPADTVEELRNII
GMDKPLSLPGFLAKFDYYMPVIAGCREAIKRIAYEFVEMKAKEGVVYVEV
RYSPHLLANSKVDPMPWNQTEGDVTPDDVVDLVNQGLQEGEQAFGIKVRS
ILCCMRHQPSWSLEVLELCKKYNQKTVVAMDLAGDETIEGSSLFPGHVEA
YEGAVKNGIHRTVHAGEVGSPEVVREAVDILKTERVGHGYHTIEDEALYN
RLLKENMHFEVCPWSSYLTGAWDPKTTHAVVRFKNDKANYSLNTDDPLIF
KSTLDTDYQMTKKDMGFTEEEFKRLNINAAKSSFLPEEEKKELLERLYRE
YQ

Cytidine deaminase is an enzyme that in humans is encoded by the CDA gene, which has the following mRNA sequence:

Homo sapiens cytidine deaminase (CDA), mRNA
(SEQ ID NO: 5; NM_001785.3):
CCCGCTGCTCTGCTGCCTGCCCGGGGTACCAACATGGCCCAGAAGCGTC
CTGCCTGCACCCTGAAGCCTGAGTGTGTCCAGCAGCTGCTGGTTTGCTC
CCAGGAGGCCAAGAAGTCAGCCTACTGCCCCTACAGTCACTTTCCTGTG
GGGGCTGCCCTGCTCACCCAGGAGGGGAGAATCTTCAAAGGGTGCAACA
TAGAAAATGCCTGCTACCCGCTGGGCATCTGTGCTGAACGGACCGCTAT
CCAGAAGGCCGTCTCAGAAGGGTACAAGGATTTCAGGGCAATTGCTATC
GCCAGTGACATGCAAGATGATTTTATCTCTCCATGTGGGGCCTGCAGGC
AAGTCATGAGAGAGTTTGGCACCAACTGGCCCGTGTACATGACCAAGCC
GGATGGTACGTATATTGTCATGACGGTCCAGGAGCTGCTGCCCTCCTCC
TTTGGGCCTGAGGACCTGCAGAAGACCCAGTGACAGCCAGAGAATGCCC
ACTGCCTGTAACAGCCACCTGGAGAACTTCATAAAGATGTCTCACAGCC
CTGGGGACACCTGCCCAGTGGGCCCCAGCCCTACAGGGACTGGGCAAAG
ATGATGTTTCCAGATTACACTCCAGCCTGAGTCAGCACCCCTCCTAGCA
ACCTGCCTTGGGACTTAGAACACCGCCGCCCCCTGCCCCACCTTTCCTT
TCCTTCCTGTGGGCCCTCTTTCAAAGTCCAGCCTAGTCTGGACTGCTTC
CCCATCAGCCTTCCCAAGGTTCTATCCTGTTCCGAGCAACTTTTCTAAT
TATAAACATCACAGAACATCCTGGA

The human CDA-encoded protein is:

Homo sapiens cytidine deaminase (CDA), protein
(SEQ ID NO: 6; NP_001776.1)
MAQKRPACTLKPECVQQLLVCSQEAKKSAYCPYSHFPVGAALLTQEGRI
FKGCNIENACYPLGICAERTAIQKAVSEGYKDFRAIAIASDMQDDFISP
CGACRQVMREFGTNWPVYMTKPDGTYIVMTVQELLPSSFGPEDLQKTQ

The cytidine deaminase gene encodes for an enzyme involved in pyrimidine salvaging. The encoded protein forms a homotetramer that catalyzes the irreversible hydrolytic deamination of cytidine and deoxycytidine to uridine and deoxyuridine, respectively. It is one of several deaminases responsible for maintaining the cellular pyrimidine pool. Mutations in this gene have been described as associated with decreased sensitivity to the cytosine nucleoside analogue cytosine arabinoside, used in the treatment of certain childhood leukemias. Apobec-1 is an RNA-specific cytidine deaminase that possesses homology to other members of the cytidine/deoxycytidine deaminase family, particularly within the domain HVE-PCXXC proposed to coordinate zinc binding and catalysis. APOBEC1 (rat) is an apolipoprotein B mRNA editing enzyme. The APOBEC1 protein is responsible for the postranscriptional editing of a CAA codon for Gln to a UAA codon for a stop codon in the APOB mRNA. APOBEC1 has also been described as involved in CGA (Arg) to UGA (Stop) editing in the NF1 mRNA. APOBEC1 has been described to be expressed exclusively in the small intestine. The rat apobec-1 gene spans 16 kb and includes one untranslated (exon A) and five translated exons (exons 1-5).

The wild-type mRNA sequence of rat APOBEC1 is the following:

Rattus norvegicus apolipoprotein B mRNA editing
enzyme catalytic subunit 1 (Apobec1), mRNA
(SEQ ID NO: 3; NM_012907.2)
CCAAGGTCCTGCTTTTGCATCTTAAGCCGCCCCTCCTTTCTCCAACAGA
CACGAGGAGCAAAGGGTAACTGAGAGGGAGTAGCAGGTAAAGCCCACAG
TGTTCTCACCGGGTCACCCTGAGGACTTCTTAGTTATAGGAGCTGCTTC
ATTCTCTCCGATCCGTGCTGGCTTCTCTCCCACTCTCACTTGAAGGAAG
GGGAAAGCTTTCTAAGTTTAGCCGTCACTCTGGAATTTAACATCATCGA
TGTTCTACTGTGCAGCGTTGATGGTTCGATGGGCTCTCTCCAGGGAGGA
CGGAAATCCAGATGCCACTTCCTTCTTCATTTACATAGCATTCATATCA
CGTCGCGACTGACGCTCAGGAATGAGTCATCCTGTGTCCCTGCAGGTGG
CCGTGGGCACACCTGAGGAAGCAAAGTCCGGCACGCAGCTGGCAGCAGC
CATCGCCGCAACATAAGCTCCCGAGGAAGGAGTCCAGAGACACAGAGAG
CAAGATGAGTTCCGAGACAGGCCCTGTAGCTGTTGATCCCACTCTGAGG
AGAAGAATTGAGCCCCACGAGTTTGAAGTCTTCTTTGACCCCCGGGAAC
TTCGGAAAGAGACCTGTCTGCTGTATGAGATCAACTGGGGAGGAAGGCA
CAGCATCTGGCGACACACGAGCCAAAACACCAACAAACACGTTGAAGTC
AATTTCATAGAAAAATTTACTACAGAAAGATACTTTTGTCCAAACACCA
GATGCTCCATTACCTGGTTCCTGTCCTGGAGTCCCTGTGGGGAGTGCTC
CAGGGCCATTACAGAATTTTTGAGCCGATACCCCCATGTAACTCTGTTT
ATTTATATAGCACGGCTTTATCACCACGCAGATCCTCGAAATCGGCAAG
GACTCAGGGACCTTATTAGCAGCGGTGTTACTATCCAGATCATGACGGA
GCAAGAGTCTGGCTACTGCTGGAGGAATTTTGTCAACTACTCCCCTTCG
AATGAAGCTCATTGGCCAAGGTACCCCCATCTGTGGGTGAGGCTGTACG
TACTGGAACTCTACTGCATCATTTTAGGACTTCCACCCTGTTTAAATAT
TTTAAGAAGAAAACAACCTCAACTCACGTTTTTCACGATTGCTCTTCAA
AGCTGCCATTACCAAAGGCTACCACCCCACATCCTGTGGGCCACAGGGT
TGAAATGACTTCTGGGAGTTGGGGATGGATGAAATGACTCCTTGTATGT
CTTGACAGCAAGCATTGATTACCCACTAAAGAGCGACTGCCACAAGGAA
AAAAAAAAAAAAAAAAAAAAAAAAAAAAAAAAAAAAAAAAAAAAAA

The corresponding wild-type rat APOBEC1 protein sequence is the following:

Rattus norvegicus apolipoprotein B mRNA editing
enzyme catalytic subunit 1 (Apobec1), protein
(SEQ ID NO: 4; NP_037039.1)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK

Activation-induced cytidine deaminase, also known as AICDA and AID, is a 24 kDa enzyme which in humans is encoded by the AICDA gene. It creates mutations in DNA by deamination of cytosine base, which turns it into uracil (which is recognized as a thymine). In other words, it changes a C: G base pair into a U: G mismatch. The cell's DNA replication machinery recognizes the U as a T, and hence C: G is converted to a T: A base pair. During germinal center development of B lymphocytes, AID also generates other types of mutations, such as C: G to A: T.

Homo sapiens activation induced cytidine
deaminase (AICDA), transcript variant 1, mRNA
(NM_020661.4; SEQ ID NO: 15)
GTCAGACTAAGACAGAGAACCATCATTAATTGAAGTGAGATTTTTCTGG
CCTGAGACTTGCAGGGAGGCAAGAAGACACTCTGGACACCACTATGGAC
AGCCTCTTGATGAACCGGAGGAAGTTTCTTTACCAATTCAAAAATGTCC
GCTGGGCTAAGGGTCGGCGTGAGACCTACCTGTGCTACGTAGTGAAGAG
GCGTGACAGTGCTACATCCTTTTCACTGGACTTTGGTTATCTTCGCAAT
AAGAACGGCTGCCACGTGGAATTGCTCTTCCTCCGCTACATCTCGGACT
GGGACCTAGACCCTGGCCGCTGCTACCGCGTCACCTGGTTCACCTCCTG
GAGCCCCTGCTACGACTGTGCCCGACATGTGGCCGACTTTCTGCGAGGG
AACCCCAACCTCAGTCTGAGGATCTTCACCGCGCGCCTCTACTTCTGTG
AGGACCGCAAGGCTGAGCCCGAGGGGCTGCGGCGGCTGCACCGCGCCGG
GGTGCAAATAGCCATCATGACCTTCAAAGATTATTTTTACTGCTGGAAT
ACTTTTGTAGAAAACCACGAAAGAACTTTCAAAGCCTGGGAAGGGCTGC
ATGAAAATTCAGTTCGTCTCTCCAGACAGCTTCGGCGCATCCTTTTGCC
CCTGTATGAGGTTGATGACTTACGAGACGCATTTCGTACTTTGGGACTT
TGATAGCAACTTCCAGGAATGTCACACACGATGAAATATCTCTGCTGAA
GACAGTGGATAAAAAACAGTCCTTCAAGTCTTCTCTGTTTTTATTCTTC
AACTCTCACTTTCTTAGAGTTTACAGAAAAAATATTTATATACGACTCT
TTAAAAAGATCTATGTCTTGAAAATAGAGAAGGAACACAGGTCTGGCCA
GGGACGTGCTGCAATTGGTGCAGTTTTGAATGCAACATTGTCCCCTACT
GGGAATAACAGAACTGCAGGACCTGGGAGCATCCTAAAGTGTCAACGTT
TTTCTATGACTTTTAGGTAGGATGAGAGCAGAAGGTAGATCCTAAAAAG
CATGGTGAGAGGATCAAATGTTTTTATATCAACATCCTTTATTATTTGA
TTCATTTGAGTTAACAGTGGTGTTAGTGATAGATTTTTCTATTCTTTTC
CCTTGACGTTTACTTTCAAGTAACACAAACTCTTCCATCAGGCCATGAT
CTATAGGACCTCCTAATGAGAGTATCTGGGTGATTGTGACCCCAAACCA
TCTCTCCAAAGCATTAATATCCAATCATGCGCTGTATGTTTTAATCAGC
AGAAGCATGTTTTTATGTTTGTACAAAAGAAGATTGTTATGGGTGGGGA
TGGAGGTATAGACCATGCATGGTCACCTTCAAGCTACTTTAATAAAGGA
TCTTAAAATGGGCAGGAGGACTGTGAACAAGACACCCTAATAATGGGTT
GATGTCTGAAGTAGCAAATCTTCTGGAAACGCAAACTCTTTTAAGGAAG
TCCCTAATTTAGAAACACCCACAAACTTCACATATCATAATTAGCAAAC
AATTGGAAGGAAGTTGCTTGAATGTTGGGGAGAGGAAAATCTATTGGCT
CTCGTGGGTCTCTTCATCTCAGAAATGCCAATCAGGTCAAGGTTTGCTA
CATTTTGTATGTGTGTGATGCTTCTCCCAAAGGTATATTAACTATATAA
GAGAGTTGTGACAAAACAGAATGATAAAGCTGCGAACCGTGGCACACGC
TCATAGTTCTAGCTGCTTGGGAGGTTGAGGAGGGAGGATGGCTTGAACA
CAGGTGTTCAAGGCCAGCCTGGGCAACATAACAAGATCCTGTCTCTCAA
AAAAAAAAAAAAAAAAAAGAAAGAGAGAGGGCCGGGCGTGGTGGCTCAC
GCCTGTAATCCCAGCACTTTGGGAGGCCGAGCCGGGCGGATCACCTGTG
GTCAGGAGTTTGAGACCAGCCTGGCCAACATGGCAAAACCCCGTCTGTA
CTCAAAATGCAAAAATTAGCCAGGCGTGGTAGCAGGCACCTGTAATCCC
AGCTACTTGGGAGGCTGAGGCAGGAGAATCGCTTGAACCCAGGAGGTGG
AGGTTGCAGTAAGCTGAGATCGTGCCGTTGCACTCCAGCCTGGGCGACA
AGAGCAAGACTCTGTCTCAGAAAAAAAAAAAAAAAAGAGAGAGAGAGAG
AAAGAGAACAATATTTGGGAGAGAAGGATGGGGAAGCATTGCAAGGAAA
TTGTGCTTTATCCAACAAAATGTAAGGAGCCAATAAGGGATCCCTATTT
GTCTCTTTTGGTGTCTATTTGTCCCTAACAACTGTCTTTGACAGTGAGA
AAAATATTCAGAATAACCATATCCCTGTGCCGTTATTACCTAGCAACCC
TTGCAATGAAGATGAGCAGATCCACAGGAAAACTTGAATGCACAACTGT
CTTATTTTAATCTTATTGTACATAAGTTTGTAAAAGAGTTAAAAATTGT
TACTTCATGTATTCATTTATATTTTATATTATTTTGCGTCTAATGATTT
TTTATTAACATGATTTCCTTTTCTGATATATTGAAATGGAGTCTCAAAG
CTTCATAAATTTATAACTTTAGAAATGATTCTAATAACAACGTATGTAA
TTGTAACATTGCAGTAATGGTGCTACGAAGCCATTTCTCTTGATTTTTA
GTAAACTTTTATGACAGCAAATTTGCTTCTGGCTCACTTTCAATCAGTT
AAATAAATGATAAATAATTTTGGAAGCTGTGAAGATAAAATACCAAATA
AAATAATATAAAAGTGATTTATATGAAGTTAAAATAAAAAATCAGTATG
ATGGAATAAA
Homo sapiens activation induced cytidine
deaminase (AICDA), transcript variant 1, protein
(NP_065712.1; SEQ ID NO: 16)
MDSLLMNRRKFLYQFKNVRWAKGRRETYLCYVVKRRDSATSFSLDFGYL
RNKNGCHVELLFLRYISDWDLDPGRCYRVTWFTSWSPCYDCARHVADFL
RGNPNLSLRIFTARLYFCEDRKAEPEGLRRLHRAGVQIAIMTFKDYFYC
WNTFVENHERTFKAWEGLHENSVRLSRQLRRILLPLYEVDDLRDAFRTL
GL
The pGH335_MS2-AID*Δ-Hygro plasmid has the
following sequence
>pGH335_MS2-AID*Δ-Hygro sequence 11382 bps
(SEQ ID NO: 17)
GTCGACGGATCGGGAGATCTCCCGATCCCCTATGGTGCACTCTCAGTAC
AATCTGCTCTGATGCCGCATAGTTAAGCCAGTATCTGCTCCCTGCTTGT
GTGTTGGAGGTCGCTGAGTAGTGCGCGAGCAAAATTTAAGCTACAACAA
GGCAAGGCTTGACCGACAATTGCATGAAGAATCTGCTTAGGGTTAGGCG
TTTTGCGCTGCTTCGCGATGTACGGGCCAGATATACGCGTTGACATTGA
TTATTGACTAGTTATTAATAGTAATCAATTACGGGGTCATTAGTTCATA
GCCCATATATGGAGTTCCGCGTTACATAACTTACGGTAAATGGCCCGCC
TGGCTGACCGCCCAACGACCCCCGCCCATTGACGTCAATAATGACGTAT
GTTCCCATAGTAACGCCAATAGGGACTTTCCATTGACGTCAATGGGTGG
AGTATTTACGGTAAACTGCCCACTTGGCAGTACATCAAGTGTATCATAT
GCCAAGTACGCCCCCTATTGACGTCAATGACGGTAAATGGCCCGCCTGG
CATTATGCCCAGTACATGACCTTATGGGACTTTCCTACTTGGCAGTACA
TCTACGTATTAGTCATCGCTATTACCATGGTGATGCGGTTTTGGCAGTA
CATCAATGGGCGTGGATAGCGGTTTGACTCACGGGGATTTCCAAGTCTC
CACCCCATTGACGTCAATGGGAGTTTGTTTTGGCACCAAAATCAACGGG
ACTTTCCAAAATGTCGTAACAACTCCGCCCCATTGACGCAAATGGGCGG
TAGGCGTGTACGGTGGGAGGTCTATATAAGCAGCGCGTTTTGCCTGTAC
TGGGTCTCTCTGGTTAGACCAGATCTGAGCCTGGGAGCTCTCTGGCTAA
CTAGGGAACCCACTGCTTAAGCCTCAATAAAGCTTGCCTTGAGTGCTTC
AAGTAGTGTGTGCCCGTCTGTTGTGTGACTCTGGTAACTAGAGATCCCT
CAGACCCTTTTAGTCAGTGTGGAAAATCTCTAGCAGTGGCGCCCGAACA
GGGACTTGAAAGCGAAAGGGAAACCAGAGGAGCTCTCTCGACGCAGGAC
TCGGCTTGCTGAAGCGCGCACGGCAAGAGGCGAGGGGCGGCGACTGGTG
AGTACGCCAAAAATTTTGACTAGCGGAGGCTAGAAGGAGAGAGATGGGT
GCGAGAGCGTCAGTATTAAGCGGGGGAGAATTAGATCGCGATGGGAAAA
AATTCGGTTAAGGCCAGGGGGAAAGAAAAAATATAAATTAAAACATATA
GTATGGGCAAGCAGGGAGCTAGAACGATTCGCAGTTAATCCTGGCCTGT
TAGAAACATCAGAAGGCTGTAGACAAATACTGGGACAGCTACAACCATC
CCTTCAGACAGGATCAGAAGAACTTAGATCATTATATAATACAGTAGCA
ACCCTCTATTGTGTGCATCAAAGGATAGAGATAAAAGACACCAAGGAAG
CTTTAGACAAGATAGAGGAAGAGCAAAACAAAAGTAAGACCACCGCACA
GCAAGCGGCCGCTGATCTTCAGACCTGGAGGAGGAGATATGAGGGACAA
TTGGAGAAGTGAATTATATAAATATAAAGTAGTAAAAATTGAACCATTA
GGAGTAGCACCCACCAAGGCAAAGAGAAGAGTGGTGCAGAGAGAAAAAA
GAGCAGTGGGAATAGGAGCTTTGTTCCTTGGGTTCTTGGGAGCAGCAGG
AAGCACTATGGGCGCAGCGTCAATGACGCTGACGGTACAGGCCAGACAA
TTATTGTCTGGTATAGTGCAGCAGCAGAACAATTTGCTGAGGGCTATTG
AGGCGCAACAGCATCTGTTGCAACTCACAGTCTGGGGCATCAAGCAGCT
CCAGGCAAGAATCCTGGCTGTGGAAAGATACCTAAAGGATCAACAGCTC
CTGGGGATTTGGGGTTGCTCTGGAAAACTCATTTGCACCACTGCTGTGC
CTTGGAATGCTAGTTGGAGTAATAAATCTCTGGAACAGATTTGGAATCA
CACGACCTGGATGGAGTGGGACAGAGAAATTAACAATTACACAAGCTTA
ATACACTCCTTAATTGAAGAATCGCAAAACCAGCAAGAAAAGAATGAAC
AAGAATTATTGGAATTAGATAAATGGGCAAGTTTGTGGAATTGGTTTAA
CATAACAAATTGGCTGTGGTATATAAAATTATTCATAATGATAGTAGGA
GGCTTGGTAGGTTTAAGAATAGTTTTTGCTGTACTTTCTATAGTGAATA
GAGTTAGGCAGGGATATTCACCATTATCGTTTCAGACCCACCTCCCAAC
CCCGAGGGGACCCGACAGGCCCGAAGGAATAGAAGAAGAAGGTGGAGAG
AGAGACAGAGACAGATCCATTCGATTAGTGAACGGATCGGCACTGCGTG
CGCCAATTCTGCAGACAAATGGCAGTATTCATCCACAATTTTAAAAGAA
AAGGGGGGATTGGGGGGTACAGTGCAGGGGAAAGAATAGTAGACATAAT
AGCAACAGACATACAAACTAAAGAATTACAAAAACAAATTACAAAAATT
CAAAATTTTCGGGTTTATTACAGGGACAGCAGAGATCCAGTTTGGTTAA
TTAGCTAGCTGCAAAGATGGATAAAGTTTTAAACAGAGAGGAATCTTTG
CAGCTAATGGACCTTCTAGGTCTTGAAAGGAGTGGGAATTGGCTCCGGT
GCCCGTCAGTGGGCAGAGCGCACATCGCCCACAGTCCCCGAGAAGTTGG
GGGGAGGGGTCGGCAATTGAACCGGTGCCTAGAGAAGGTGGCGCGGGGT
AAACTGGGAAAGTGATGTCGTGTACTGGCTCCGCCTTTTTCCCGAGGGT
GGGGGAGAACCGTATATAAGTGCAGTAGTCGCCGTGAACGTTCTTTTTC
GCAACGGGTTTGCCGCCAGAACACAGGTAAGTGCCGTGTGTGGTTCCCG
CGGGCCTGGCCTCTTTACGGGTTATGGCCCTTGCGTGCCTTGAATTACT
TCCACCTGGCTGCAGTACGTGATTCTTGATCCCGAGCTTCGGGTTGGAA
GTGGGTGGGAGAGTTCGAGGCCTTGCGCTTAAGGAGCCCCTTCGCCTCG
TGCTTGAGTTGAGGCCTGGCCTGGGCGCTGGGGCCGCCGCGTGCGAATC
TGGTGGCACCTTCGCGCCTGTCTCGCTGCTTTCGATAAGTCTCTAGCCA
TTTAAAATTTTTGATGACCTGCTGCGACGCTTTTTTTCTGGCAAGATAG
TCTTGTAAATGCGGGCCAAGATCTGCACACTGGTATTTCGGTTTTTGGG
GCCGCGGGCGGCGACGGGGCCCGTGCGTCCCAGCGCACATGTTCGGCGA
GGCGGGGCCTGCGAGCGCGGCCACCGAGAATCGGACGGGGGTAGTCTCA
AGCTGGCCGGCCTGCTCTGGTGCCTGGCCTCGCGCCGCCGTGTATCGCC
CCGCCCTGGGCGGCAAGGCTGGCCCGGTCGGCACCAGTTGCGTGAGCGG
AAAGATGGCCGCTTCCCGGCCCTGCTGCAGGGAGCTCAAAATGGAGGAC
GCGGCGCTCGGGAGAGCGGGCGGGTGAGTCACCCACACAAAGGAAAAGG
GCCTTTCCGTCCTCAGCCGTCGCTTCATGTGACTCCACGGAGTACCGGG
CGCCGTCCAGGCACCTCGATTAGTTCTCGAGCTTTTGGAGTACGTCGTC
TTTAGGTTGGGGGGAGGGGTTTTATGCGATGGAGTTTCCCCACACTGAG
TGGGTGGAGACTGAAGTTAGGCCAGCTTGGCACTTGATGTAATTCTCCT
TGGAATTTGCCCTTTTTGAGTTTGGATCTTGGTTCATTCTCAAGCCTCA
GACAGTGGTTCAAAGTTTTTTTCTTCCATTTCAGGTGTCGTGACGTACG
GCCACCATGGCTTCAAACTTTACTCAGTTCGTGCTCGTGGACAATGGTG
GGACAGGGGATGTGACAGTGGCTCCTTCTAATTTCGCTAATGGGGTGGC
AGAGTGGATCAGCTCCAACTCACGGAGCCAGGCCTACAAGGTGACATGC
AGCGTCAGGCAGTCTAGTGCCCAGAAGAGAAAGTATACCATCAAGGTGG
AGGTCCCCAAAGTGGCTACCCAGACAGTGGGCGGAGTCGAACTGCCTGT
CGCCGCTTGGAGGTCCTACCTGAACATGGAGCTCACTATCCCAATTTTC
GCTACCAATTCTGACTGTGAACTCATCGTGAAGGCAATGCAGGGGCTCC
TCAAAGACGGTAATCCTATCCCTTCCGCCATCGCCGCTAACTCAGGTAT
CTACAGCGCTGGAGGAGGTGGAAGCGGAGGAGGAGGAAGCGGAGGAGGA
GGTAGCGGACCTAAGAAAAAGAGGAAGGTGGCGGCCGCTGGATCCATGG
ACAGCCTCTTGATGAACCGGAGGGAGTTTCTTTACCAATTCAAAAATGT
CCGCTGGGCTAAGGGTCGGCGTGAGACCTACCTGTGCTACGTAGTGAAG
AGGCGTGACAGTGCTACATCCTTTTCACTGGACTTTGGTTATCTTCGCA
ATAAGAACGGCTGCCACGTGGAATTGCTCTTCCTCCGCTACATCTCGGA
CTGGGACCTAGACCCTGGCCGCTGCTACCGCGTCACCTGGTTCATCTCC
TGGAGCCCCTGCTACGACTGTGCCCGACATGTGGCCGACTTTCTGCGAG
GGAACCCCAACCTCAGTCTGAGGATCTTCACCGCGCGCCTCTACTTCTG
TGAGGACCGCAAGGCTGAGCCCGAGGGGCTGCGGCGGCTGCACCGCGCC
GGGGTGCAAATAGCCATCATGACCTTCAAAGATTATTTTTACTGCTGGA
ATACTTTTGTAGAAAACCACGGAAGAACTTTCAAAGCCTGGGAAGGGCT
GCATGAAAATTCAGTTCGTCTCTCCAGACAGCTTCGGCGCATCCTTTTG
CCCCTGTATGAGGTTGATGACTTACGAGACGCATTTCGTACTTGTACAG
GCAGTGGAGAGGGCAGAGGAAGTCTGCTAACATGCGGTGACGTCGAGGA
GAATCCTGGCCCAACCATGAAAAAGCCTGAACTCACCGCTACCTCTGTC
GAGAAGTTTCTGATCGAAAAGTTCGACAGCGTCTCCGACCTGATGCAGC
TCTCCGAGGGCGAAGAATCTCGGGCTTTCAGCTTCGATGTGGGAGGGCG
TGGATATGTCCTGCGGGTGAATAGCTGCGCCGATGGTTTCTACAAAGAT
CGCTATGTTTATCGGCACTTTGCATCCGCCGCTCTCCCTATTCCCGAAG
TGCTTGACATTGGGGAGTTCAGCGAGAGCCTGACCTATTGCATCTCCCG
CCGTGCACAGGGTGTCACCTTGCAAGACCTGCCTGAAACCGAACTGCCC
GCTGTTCTCCAGCCCGTCGCCGAGGCCATGGATGCCATCGCTGCCGCCG
ATCTTAGCCAGACCAGCGGGTTCGGCCCATTCGGACCTCAAGGAATCGG
TCAATACACTACATGGCGCGATTTCATCTGCGCTATTGCTGATCCCCAT
GTGTATCACTGGCAAACTGTGATGGACGACACCGTCAGTGCCTCCGTCG
CCCAGGCTCTCGATGAGCTGATGCTTTGGGCCGAGGACTGCCCCGAAGT
CCGGCACCTCGTGCACGCCGATTTCGGCTCCAACAATGTCCTGACCGAC
AATGGCCGCATAACAGCCGTCATTGACTGGAGCGAGGCCATGTTCGGGG
ATTCCCAATACGAGGTCGCCAACATCTTCTTCTGGAGGCCCTGGTTGGC
TTGTATGGAGCAGCAGACCCGCTACTTCGAGCGGAGGCATCCCGAGCTT
GCAGGATCTCCTCGGCTCCGGGCTTATATGCTCCGCATTGGTCTTGACC
AACTCTATCAGAGCTTGGTTGACGGCAATTTCGATGATGCAGCTTGGGC
TCAGGGTCGCTGCGACGCAATCGTCCGGTCCGGAGCCGGGACTGTCGGG
CGTACACAAATCGCCCGCAGAAGCGCTGCCGTCTGGACCGATGGCTGTG
TGGAAGTGCTCGCCGATAGTGGAAACAGACGCCCCAGCACTCGTCCTAG
GGCAAAGGATCTGCAGTAATGAGAATTCGATATCAAGCTTATCGGTAAT
CAACCTCTGGATTACAAAATTTGTGAAAGATTGACTGGTATTCTTAACT
ATGTTGCTCCTTTTACGCTATGTGGATACGCTGCTTTAATGCCTTTGTA
TCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAAC
GTGGCGTGGTGTGCACTGTGTTTGCTGACGCAACCCCCACTGGTTGGGG
CATTGCCACCACCTGTCAGCTCCTTTCCGGGACTTTCGCTTTCCCCCTC
CCTATTGCCACGGCGGAACTCATCGCCGCCTGCCTTGCCCGCTGCTGGA
CAGGGGCTCGGCTGTTGGGCACTGACAATTCCGTGGTGTTGTCGGGGAA
ATCATCGTCCTTTCCTTGGCTGCTCGCCTGTGTTGCCACCTGGATTCTG
CGCGGGACGTCCTTCTGCTACGTCCCTTCGGCCCTCAATCCAGCGGACC
TTCCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGCAT
CGATACCGTCGACCTCGAGACCTAGAAAAACATGGAGCAATCACAAGTA
GCAATACAGCAGCTACCAATGCTGATTGTGCCTGGCTAGAAGCACAAGA
GGAGGAGGAGGTGGGTTTTCCAGTCACACCTCAGGTACCTTTAAGACCA
ATGACTTACAAGGCAGCTGTAGATCTTAGCCACTTTTTAAAAGAAAAGG
GGGGACTGGAAGGGCTAATTCACTCCCAACGAAGACAAGATATCCTTGA
TCTGTGGATCTACCACACACAAGGCTACTTCCCTGATTGGCAGAACTAC
ACACCAGGGCCAGGGATCAGATATCCACTGACCTTTGGATGGTGCTACA
AGCTAGTACCAGTTGAGCAAGAGAAGGTAGAAGAAGCCAATGAAGGAGA
GAACACCCGCTTGTTACACCCTGTGAGCCTGCATGGGATGGATGACCCG
GAGAGAGAAGTATTAGAGTGGAGGTTTGACAGCCGCCTAGCATTTCATC
ACATGGCCCGAGAGCTGCATCCGGACTGTACTGGGTCTCTCTGGTTAGA
CCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTC
TGTTGTGTGACTCTGGTAACTAGAGATCCCTCAGACCCTTTTAGTCAGT
GTGGAAAATCTCTAGCAGGGCCCGTTTAAACCCGCTGATCAGCCTCGAC
TGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATG
AGGAAATTGCATCGCATTGTCTGAGTAGGTGTCATTCTATTCTGGGGGG
TGGGGTGGGGCAGGACAGCAAGGGGGAGGATTGGGAAGACAATAGCAGG
CATGCTGGGGATGCGGTGGGCTCTATGGCTTCTGAGGCGGAAAGAACCA
GCTGGGGCTCTAGGGGGTATCCCCACGCGCCCTGTAGCGGCGCATTAAG
CGCGGCGGGTGTGGTGGTTACGCGCAGCGTGACCGCTACACTTGCCAGC
GCCCTAGCGCCCGCTCCTTTCGCTTTCTTCCCTTCCTTTCTCGCCACGT
TCGCCGGCTTTCCCCGTCAAGCTCTAAATCGGGGGCTCCCTTTAGGGTT
CCGATTTAGTGCTTTACGGCACCTCGACCCCAAAAAACTTGATTAGGGT
GATGGTTCACGTAGTGGGCCATCGCCCTGATAGACGGTTTTTCGCCCTT
TGACGTTGGAGTCCACGTTCTTTAATAGTGGACTCTTGTTCCAAACTGG
AACAACACTCAACCCTATCTCGGTCTATTCTTTTGATTTATAAGGGATT
TTGCCGATTTCGGCCTATTGGTTAAAAAATGAGCTGATTTAACAAAAAT
TTAACGCGAATTAATTCTGTGGAATGTGTGTCAGTTAGGGTGTGGAAAG
TCCCCAGGCTCCCCAGCAGGCAGAAGTATGCAAAGCATGCATCTCAATT
AGTCAGCAACCAGGTGTGGAAAGTCCCCAGGCTCCCCAGCAGGCAGAAG
TATGCAAAGCATGCATCTCAATTAGTCAGCAACCATAGTCCCGCCCCTA
ACTCCGCCCATCCCGCCCCTAACTCCGCCCAGTTCCGCCCATTCTCCGC
CCCATGGCTGACTAATTTTTTTTATTTATGCAGAGGCCGAGGCCGCCTC
TGCCTCTGAGCTATTCCAGAAGTAGTGAGGAGGCTTTTTTGGAGGCCTA
GGCTTTTGCAAAAAGCTCCCGGGAGCTTGTATATCCATTTTCGGATCTG
ATCAGCACGTGTTGACAATTAATCATCGGCATAGTATATCGGCATAGTA
TAATACGACAAGGTGAGGAACTAAACCATGGCCAAGTTGACCAGTGCCG
TTCCGGTGCTCACCGCGCGCGACGTCGCCGGAGCGGTCGAGTTCTGGAC
CGACCGGCTCGGGTTCTCCCGGGACTTCGTGGAGGACGACTTCGCCGGT
GTGGTCCGGGACGACGTGACCCTGTTCATCAGCGCGGTCCAGGACCAGG
TGGTGCCGGACAACACCCTGGCCTGGGTGTGGGTGCGCGGCCTGGACGA
GCTGTACGCCGAGTGGTCGGAGGTCGTGTCCACGAACTTCCGGGACGCC
TCCGGGCCGGCCATGACCGAGATCGGCGAGCAGCCGTGGGGGCGGGAGT
TCGCCCTGCGCGACCCGGCCGGCAACTGCGTGCACTTCGTGGCCGAGGA
GCAGGACTGACACGTGCTACGAGATTTCGATTCCACCGCCGCCTTCTAT
GAAAGGTTGGGCTTCGGAATCGTTTTCCGGGACGCCGGCTGGATGATCC
TCCAGCGCGGGGATCTCATGCTGGAGTTCTTCGCCCACCCCAACTTGTT
TATTGCAGCTTATAATGGTTACAAATAAAGCAATAGCATCACAAATTTC
ACAAATAAAGCATTTTTTTCACTGCATTCTAGTTGTGGTTTGTCCAAAC
TCATCAATGTATCTTATCATGTCTGTATACCGTCGACCTCTAGCTAGAG
CTTGGCGTAATCATGGTCATAGCTGTTTCCTGTGTGAAATTGTTATCCG
CTCACAATTCCACACAACATACGAGCCGGAAGCATAAAGTGTAAAGCCT
GGGGTGCCTAATGAGTGAGCTAACTCACATTAATTGCGTTGCGCTCACT
GCCCGCTTTCCAGTCGGGAAACCTGTCGTGCCAGCTGCATTAATGAATC
GGCCAACGCGCGGGGAGAGGCGGTTTGCGTATTGGGCGCTCTTCCGCTT
CCTCGCTCACTGACTCGCTGCGCTCGGTCGTTCGGCTGCGGCGAGCGGT
ATCAGCTCACTCAAAGGCGGTAATACGGTTATCCACAGAATCAGGGGAT
AACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGA
CGAGCATCACAAAAATCGACGCTCAAGTCAGAGGTGGCGAAACCCGACA
GGACTATAAAGATACCAGGCGTTTCCCCCTGGAAGCTCCCTCGTGCGCT
CTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAA
CCCGGTAAGACACGACTTATCGCCACTGGCAGCAGCCACTGGTAACAGG
ATTAGCAGAGCGAGGTATGTAGGCGGTGCTACAGAGTTCTTGAAGTGGT
GGCCTAACTACGGCTACACTAGAAGAACAGTATTTGGTATCTGCGCTCT
GCTGAAGCCAGTTACCTTCGGAAAAAGAGTTGGTAGCTCTTGATCCGGC
AAACAAACCACCGCTGGTAGCGGTGGTTTTTTTGTTTGCAAGCAGCAGA
TTACGCGCAGAAAAAAAGGATCTCAAGAAGATCCTTTGATCTTTTCTAC
GGGGTCTGACGCTCAGTGGAACGAAAACTCACGTTAAGGGATTTTGGTC
ATGAGATTATCAAAAAGGATCTTCACCTAGATCCTTTTAAATTAAAAAT
GAAGTTTTAAATCAATCTAAAGTATATATGAGTAAACTTGGTCTGACAG
TTACCAATGCTTAATCAGTGAGGCACCTATCTCAGCGATCTGTCTATTT
CGTTCATCCATAGTTGCCTGACTCCCCGTCGTGTAGATAACTACGATAC
GGGAGGGCTTACCATCTGGCCCCAGTGCTGCAATGATACCGCGAGACCC
ACGCTCACCGGCTCCAGATTTATCAGCAATAAACCAGCCAGCCGGAAGG
GCCGAGCGCAGAAGTGGTCCTGCAACTTTATCCGCCTCCATCCAGTCTA
TTAATTGTTGCCGGGAAGCTAGAGTAAGTAGTTCGCCAGTTAATAGTTT
GCGCAACGTTGTTGCCATTGCTACAGGCATCGTGGTGTCACGCTCGTCG
TTTGGTATGGCTTCATTCAGCTCCGGTTCCCAACGATCAAGGCGAGTTA
CATGATCCCCCATGTTGTGCAAAAAAGCGGTTAGCTCCTTCGGTCCTCC
GATCGTTGTCAGAAGTAAGTTGGCCGCAGTGTTATCACTCATGGTTATG
GCAGCACTGCATAATTCTCTTACTGTCATGCCATCCGTAAGATGCTTTT
CTGTGACTGGTGAGTACTCAACCAAGTCATTCTGAGAATAGTGTATGCG
GCGACCGAGTTGCTCTTGCCCGGCGTCAATACGGGATAATACCGCGCCA
CATAGCAGAACTTTAAAAGTGCTCATCATTGGAAAACGTTCTTCGGGGC
GAAAACTCTCAAGGATCTTACCGCTGTTGAGATCCAGTTCGATGTAACC
CACTCGTGCACCCAACTGATCTTCAGCATCTTTTACTTTCACCAGCGTT
TCTGGGTGAGCAAAAACAGGAAGGCAAAATGCCGCAAAAAAGGGAATAA
GGGCGACACGGAAATGTTGAATACTCATACTCTTCCTTTTTCAATATTA
TTGAAGCATTTATCAGGGTTATTGTCTCATGAGCGGATACATATTTGAA
TGTATTTAGAAAAATAAACAAATAGGGGTTCCGCGCACATTTCCCCGAA
AAGTGCCACCTGAC

Within the above plasmid, AID*Δ includes the following peptide sequence (SEQ ID NO: 18):

MDSLLMNRREFLYQFKNVRWAKGRRETYLCYVVKRRDSATSFSLDFGYL
RNKNGCHVELLFLRYISDWDLDPGRCYRVTWFISWSPCYDCARHVADFL
RGNPNLSLRIFTARLYFCEDRKAEPEGLRRLHRAGVQIAIMTFKDYFYC
WNTFVENHGRTFKAWEGLHENSVRLSRQLRRILLPLYEVDDLRDAFRT

The above plasmid also includes the AID*4 DNA sequence (SEQ ID NO: 30):

ATGGACAGCCTCTTGATGAACCGGAGGGAGTTTCTTTACCAATTCAAAA
ATGTCCGCTGGGCTAAGGGTCGGCGTGAGACCTACCTGTGCTACGTAGT
GAAGAGGCGTGACAGTGCTACATCCTTTTCACTGGACTTTGGTTATCTT
CGCAATAAGAACGGCTGCCACGTGGAATTGCTCTTCCTCCGCTACATCT
CGGACTGGGACCTAGACCCTGGCCGCTGCTACCGCGTCACCTGGTTCAT
CTCCTGGAGCCCCTGCTACGACTGTGCCCGACATGTGGCCGACTTTCTG
CGAGGGAACCCCAACCTCAGTCTGAGGATCTTCACCGCGCGCCTCTACT
TCTGTGAGGACCGCAAGGCTGAGCCCGAGGGGCTGCGGCGGCTGCACCG
CGCCGGGGTGCAAATAGCCATCATGACCTTCAAAGATTATTTTTACTGC
TGGAATACTTTTGTAGAAAACCACGGAAGAACTTTCAAAGCCTGGGAAG
GGCTGCATGAAAATTCAGTTCGTCTCTCCAGACAGCTTCGGCGCATCCT
TTTGCCCCTGTATGAGGTTGATGACTTACGAGACGCATTTCGTACT

Guanine deaminase—also known as cypin, guanase, guanine aminase, GAH, and guanine aminohydrolase—is an aminohydrolase enzyme which converts guanine to xanthine. Cypin is a major cytosolic protein that interacts with PSD-95.

Homo sapiens guanine deaminase (GDA), transcript
variant 2, mRNA
(NM_004293.4; SEQ ID NO: 19)
AGAAAAATCCTATTGGCATTGAGGAGGTAGGGAGCCAGCCCCTGGGCGC
GGCCTGCAGGGTACCGGCAACCGCCCGGGTAAGCGGGGGCAGGACAAGG
CCGGAGCCTGTGTCCGCCCGGCAGCCGCCCGCAGCTGCAGAGAGTCCCG
CTGCGTCTCCGCCGCGTGCGCCCTCCTCGACCAGCAGACCCGCGCTGCG
CTCCGCCGCTGACATGTGTGCCGCTCAGATGCCGCCCCTGGCGCACATC
TTCCGAGGGACGTTCGTCCACTCCACCTGGACCTGCCCCATGGAGGTGC
TGCGGGATCACCTCCTCGGCGTGAGCGACAGCGGCAAAATAGTGTTTTT
AGAAGAAGCATCTCAACAGGAAAAACTGGCCAAAGAATGGTGCTTCAAG
CCGTGTGAAATAAGAGAACTGAGCCACCATGAGTTCTTCATGCCTGGGC
TGGTTGATACACACATCCATGCCTCTCAGTATTCCTTTGCTGGAAGTAG
CATAGACCTGCCACTCTTGGAGTGGCTGACCAAGTACACATTTCCTGCA
GAACACAGATTCCAGAACATCGACTTTGCAGAAGAAGTATATACCAGAG
TTGTCAGGAGAACACTAAAGAATGGAACAACCACAGCTTGTTACTTTGC
AACAATTCACACTGACTCATCTCTGCTCCTTGCCGACATTACAGATAAA
TTTGGACAGCGGGCATTTGTGGGCAAAGTTTGCATGGATTTGAATGACA
CTTTTCCAGAATACAAGGAGACCACTGAGGAATCGATCAAGGAAACTGA
GAGATTTGTGTCAGAAATGCTCCAAAAGAACTATTCTAGAGTGAAGCCC
ATAGTGACACCACGTTTTTCCCTCTCCTGCTCTGAGACTTTGATGGGTG
AACTGGGCAACATTGCTAAAACCCGTGATTTGCACATTCAGAGCCATAT
AAGTGAAAATCGTGATGAAGTTGAAGCTGTGAAAAACTTATACCCCAGT
TATAAAAACTACACATCTGTGTATGATAAAAACAATCTTTTGACAAATA
AGACAGTGATGGCACACGGCTGCTACCTCTCTGCAGAAGAACTGAACGT
ATTCCATGAACGAGGAGCATCCATCGCACACTGTCCCAATTCTAATTTA
TCGCTCAGCAGTGGATTTCTAAATGTGCTAGAAGTCCTGAAACATGAAG
TCAAGATAGGGCTGGGTACAGACGTGGCTGGTGGCTATTCATATTCCAT
GCTTGATGCAATCAGAAGAGCAGTGATGGTTTCCAATATCCTTTTAATT
AATAAGGTAAATGAGAAAAGCCTCACCCTCAAAGAAGTCTTCAGACTAG
CTACTCTTGGAGGAAGCCAAGCCCTGGGGCTGGATGGTGAGATTGGAAA
CTTTGAAGTGGGCAAGGAATTTGATGCCATCCTGATCAACCCCAAAGCA
TCCGACTCTCCCATTGACCTGTTTTATGGGGACTTTTTTGGTGATATTT
CTGAGGCTGTTATCCAGAAGTTCCTCTATCTAGGAGATGATCGAAATAT
TGAAGAGGTTTATGTGGGCGGAAAGCAGGTGGTTCCGTTTTCCAGCTCA
GTGTAAGACCCTCGGGCGTCTACAAAGTTCTCCTGGGATTAGCGTGGTT
CTGCATCTCCCTTGTGCCCAGGTGGAGTTAGAAAGTCAAAAAATAGTAC
CTTGTTCTTGGGATGACTATCCCTTTCTGTGTCTAGTTACAGTATTCAC
TTGACAAATAGTTCGAAGGAAGTTGCACTAATTCTCAACTCTGGTTGAG
AGGGTTCATAAATTTCATGAAAATATCTCCCTTTGGAGCTGCTCAGACT
TACTTTAAGCTCAAACAGAAGGGAATGCTATTACTGGTGGTGTTCCTAC
GGTAAGACTTAAGCAAAGCCTTTTTCATATTTGAAAATGTGGAAAGAAA
AGATGTTCCTAAAAGGTTAGATATTTTGAGCTAATAATTGCAAAAATTA
GAAGACTGAAAATGGACCCATGAGAGTATATTTTTATGAGGGAGCAAAA
GTTAGACTGAGAACAAACGTTAGAAAATCACTTCAGATTGTGTTTGAAA
ATTATATACTGAGCATACTAATTTAAAAAGAGAACTTGTTGAAATTTAA
AACGTGTTTCTAGGTTGACCTTGTGTTTTAGAAATTTGCACTTAATGGA
ATTTGCATTTCAGAGATGTGTTAGTGTTGTGCTTTGCCTTCTTTGGCGA
TGAATGTCAGAAATTGAATGCCACATGCTTTCATAATATAGTTTTGTGC
TTCAAAGTGTTTGACAGAAGTTGGGTATTAAAGATTTAAAGTCTCTTAG
GAATATTATTCATGTAACTCCATGGCATAAATAGTTGTATTTTTGTGTA
CTTTAAAATCAACTTATAACTGTGAGATGTTATTGCTTCCATTTTATTA
GAAGAGAAACAAATTCCATGCTTTATGGAATTTATGTAGACTGGAGTCT
TCGTGAACTGGGGCAAATGCTGGCATCCAGGAGCCGCCAATACTAACAG
GACAGGTTCCATTGCCATGGCCTATTCCACCCAAACAATATGTTGTAGT
TTCTGGAAATTCCATACTCAGATATCAGTCTGCTAGAACTTTAAAATGA
AGGACAAATCCTGTTAAAGAAATATTGTTAAAAATCTTTAAACCCTGTG
TATTGAAAGCACTCTATTTTCTAATTTTATCCAGTTTTCTGTTTAACTC
CTTATAATGTTTAGGATATTAAAATTTTAGGATAATGAAGAGTACATAA
TGTCCTACTTAATATTTATGTTAATAGGACTTAATTCTTACTAGACATC
TAGGAACATTACAAAGCAAAGACTATTTTTATGCTTCCATAACCTAGAA
TTAAAACCAAATTATGACCTTATGATAAATCTTTAAGTATTGGTGTGAA
TGTTATTTAAATTCTATATTTTTCTTATTTAATTACAAATACTATAAAT
GAGCAAGGAAAAGGAATAGACTTTCTTAATATATTATAACACTCATTCC
TAGAGCTTAGGGGTGACTCTTTAATATTACCTTATAGTAGAAACTTTAT
GTAATATAGCTAACTCCGTATTTACAGAACAAAAAAACACAGTTCCCCC
TCCTGTAGTATAAATTTTATTTTCACATACTTAGCTAATTTAGCAGTAA
TTGGCCCAGTTTTTTCCCTAATAGAAATACTTTTAGATTTGATTATGTA
TACATGACACCTAAAGAGGGAACAAAAGTTAGTTTTATTTTTTTAATAA
ACAACAGAGTTTGTTTTGTGAGATAAGTATCTTAGTAAACCCAATTTCC
AGTCTTAGTCTGTATTTCCAATATTTCTAATTCCTGAGCCACGTCAAAG
ATGCCTTGCCAAATTTCTCCCCATTTCTCTACGGGGCTAGCAAAAATCT
TCAGCTTTATCACTCAACCCCTGCCAAAGGAACTTGATTACATGGTGTC
TAACCAAATGAGCAGGCTTAGGAATTTAGATGAGATGTGTAAGATTCAC
TTACAGGCAGTAGCTGCTTCTAGCATTTGCAAGATCCTACACTTTTACC
TTCTTTAAGGGTGTACATTTTGATGTTGAACATCAGTTTTCATGTAGAC
TTAGGACTCATGTGCAGTAAATATAAATAAGTGTAGCATCAGAAGCAGT
AGGAATGGCCGTATACAACCATCCTGTTAAACATTTAAATTTAGCTCTG
ATAGTGTGTTAAGACCTGAATATCTTTCCTAGTAAAAATAGGATGTGTT
GAAATATTTATATGTACTTTGATCTCTCCACATCACTTATAACTTATGT
GTTTTATTTCTCCAAGTGCGGTGTTCCTGAATGTTATGTATGCTTTTTT
TTCTGTACCACAGGCATTATCTATACCTGGGGCCAGATTTTCTGCACTT
TGAAATGTTGCCTTTGCCTAATGTAGGTTGACTTTCTGAATTGTGGAGA
GGCACTTTTCCAAGCCAATCTTATTTGTCACTTTTTGTTTTAATATCTT
GCTCTCTGACAGGAAAGAAACAATTCACTTACCAGCCTCCTCACCCCAT
CCTCCACCATTTCCTTAATGTTCCATGGTATTTTCAACGGAATACACTT
TGAAAGGTAAAAACAATTCAAAAGTATCGATTATCATAAATTCACAAAA
TATTTTTGCAACCAGAACACAAAAGCAGGCTAGTCAGCTAAGGTAAATT
TCATTTTCAAACGAGAGGGAAACATGGGAAGTAAAAGATTAGGATGTGA
AAGGTTGTCCTAAACAGACCAAGGAGACTGTTCCCTAATTTATTCTCTT
GGCTGGTTCTCTCATTGAATTATCAGACCCCAAGAGGAGATATTGGAAC
AGGCTCCCTTCATGCCAAGGGTCTTTCTAAGTTAATACTGTGAGCATTG
AGCCCCCATTAAAACTCTTTTTTACTTCAGAAAGAATTTTACAGGTTAA
AGGGAAAGAAATGGTGGGAAACTCTCCCCGTAATGCTTAGCCAACTTTA
AAGTGTACCCTTCAATATCCCCATTGGCAACTGCAGCTGAGATCTTAGA
GAGGAAATATAACCGGTGTGAGATCTAGCAATGCATTTTGAATCTTCAC
TCCCTACCAGGCTCTTCCTATTTTTAATCTCTTCACCTCAGAACTAGAC
ATATGGAGAGCTTTAAAGGCAAGCTGGAAGGCACATTGTATCAATTCTA
CCTTGTGCTATACGTAGGAGAGATCCAAAATTTGGATGCTTCTGGAGAC
TCTTAGACATCTTTTCATTGTTGTCCATTTTTAAAGTTGATGATTGCTG
GAAACATTCACACGCTTAAAAGCAATGGTGTGAGTTATTAATGGGTAAA
CTAAGAAGTGTTATAGGCAATGACTTGAAATGGTTTTTAAATTGTATGG
ATTGTTAAGAATTGTTGAAAAAAAATTTTTTTTTTTTGGACAGCTTCAA
GGAGATGTTAGCAATTTCAGATATACTAGCCAGTTTAGGTATGACTTTG
GAAGTGCAGAAACAGAAGGATACTGTTAGAAAATCCTAACATTGGTCTC
CGTGCATGTGTTCACACCTGGTCTCACTGCCTTTCCTTCCCACAGACCT
GAGTGTGAAAGACTGAGAGTTGAGGAGTTACTTTGTGGATCTTGTCCAA
ATTTAGTGAAATGTGGAAGTCAACCAGACCAATGATGGAATTAAATGTA
AATTCCAAGAGGGCTTTCACAGTCCACAGGGTTCAAATGACTTGGGTAA
CAGAAGTTATTCTTAGCTTACCTGTTATGTGACAGTGATTTACCTGTCC
ATTTCCAACCCAAAAGCCTGTCAGAAAGCATTCTTTAGAGAAAACCACT
TTACATTTGTTGTTAAACTCCTGATCGCTACTCTTAAGAATATACATGT
ATGTATTCATAGGAACATTTTTTCTCAATATTTGTATGATTCGCTTACT
GTTATTGTGCTGAGTGAGCTCCTGTGTGCTTCAGACAAAAATAAATGAG
ACTTTGTGTTTACGTTAAAAAAAAAAAAAAAAAAAAAA
Homo sapiens guanine deaminase (GDA), transcript
variant 2, protein
(NP_004284.1; SEQ ID NO: 20)
MCAAQMPPLAHIFRGTFVHSTWTCPMEVLRDHLLGVSDSGKIVFLEEAS
QQEKLAKEWCFKPCEIRELSHHEFFMPGLVDTHIHASQYSFAGSSIDLP
LLEWLTKYTFPAEHRFQNIDFAEEVYTRVVRRTLKNGTTTACYFATIHT
DSSLLLADITDKFGQRAFVGKVCMDLNDTFPEYKETTEESIKETERFVS
EMLQKNYSRVKPIVTPRFSLSCSETLMGELGNIAKTRDLHIQSHISENR
DEVEAVKNLYPSYKNYTSVYDKNNLLTNKTVMAHGCYLSAEELNVFHER
GASIAHCPNSNLSLSSGFLNVLEVLKHEVKIGLGTDVAGGYSYSMLDAI
RRAVMVSNILLINKVNEKSLTLKEVFRLATLGGSQALGLDGEIGNFEVG
KEFDAILINPKASDSPIDLFYGDFFGDISEAVIQKFLYLGDDRNIEEVY
VGGKQVVPFSSSV

Other sequences relevant to the instant disclosure include the following:

Hyperactive AID*Δ-T7 RNA Polymerase (w/o T7 promoter)-
NLS plasmid DNA sequence (SEQ ID NO: 31):
ATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTA
GTCATCGCTATTACCATGGTGATGCGGTTTTGGCAGTACATCAATGGGCGTGGATA
GCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCA
TTGACGCAAATGGGCGGTAGGCGTGTACGGTGGGAGGTCTATATAAGCAGAGCTG
GTTTAGTGAACCGTCAGATCCGCTAGAGATCCGCGGCCGCGAGAGCCGCCACCAT
GGACAGCCTCTTGATGAACCGGAGGGAGTTTCTTTACCAATTCAAAAATGTCCGCT
GGGCTAAGGGTCGGCGTGAGACCTACCTGTGCTACGTAGTGAAGAGGCGTGACAG
TGCTACATCCTTTTCACTGGACTTTGGTTATCTTCGCAATAAGAACGGCTGCCACGT
GGAATTGCTCTTCCTCCGCTACATCTCGGACTGGGACCTAGACCCTGGCCGCTGCT
ACCGCGTCACCTGGTTCATCTCCTGGAGCCCCTGCTACGACTGTGCCCGACATGTG
GCCGACTTTCTGCGAGGGAACCCCAACCTCAGTCTGAGGATCTTCACCGCGCGCCT
CTACTTCTGTGAGGACCGCAAGGCTGAGCCCGAGGGGCTGCGGCGGCTGCACCGC
GCCGGGGTGCAAATAGCCATCATGACCTTCAAAGATTATTTTTACTGCTGGAATAC
TTTTGTAGAAAACCACGGAAGAACTTTCAAAGCCTGGGAAGGGCTGCATGAAAAT
TCAGTTCGTCTCTCCAGACAGCTTCGGCGCATCCTTTTGCCCCTGTATGAGGTTGAT
GACTTACGAGACGCATTTCGTACTAGCGGCAGCGAGACTCCCGGGACCTCAGAGT
CCGCCACACCCGAAAGTAACACCATCAACATTGCTAAGAACGACTTCTCAGACAT
AGAGCTCGCGGCTATTCCGTTCAACACCCTGGCTGACCACTACGGCGAGAGACTCG
CTAGGGAGCAGCTGGCGTTGGAGCATGAATCCTACGAGATGGGCGAGGCTAGGTT
CCGCAAGATGTTCGAGCGACAATTGAAGGCAGGGGAGGTGGCGGACAACGCTGCC
GCCAAGCCCCTGATCACAACCTTGCTGCCCAAAATGATCGCGCGGATCAACGATTG
GTTTGAGGAGGTTAAGGCAAAACGGGGCAAACGCCCGACCGCATTTCAATTCCTC
CAAGAAATCAAGCCTGAGGCTGTTGCCTACATCACTATCAAGACGACACTGGCGT
GTCTCACAAGCGCCGACAACACCACCGTGCAAGCCGTCGCCAGCGCCATCGGGCG
GGCAATTGAGGATGAGGCACGGTTTGGTAGGATCCGAGACCTGGAAGCGAAGCAC
TTCAAGAAGAACGTGGAAGAGCAGTTGAACAAACGCGTCGGCCACGTGTATAAAA
AGGCTTTCATGCAGGTGGTGGAGGCCGATATGCTCAGTAAGGGGCTGCTTGGGGG
GGAGGCGTGGTCATCCTGGCACAAGGAGGATAGCATTCACGTGGGGGTCCGATGT
ATCGAGATGCTGATAGAGAGCACCGGAATGGTCTCCCTCCATCGCCAGAACGCTG
GGGTCGTAGGGCAGGACTCCGAGACTATTGAGCTGGCCCCCGAGTATGCCGAAGC
AATCGCTACACGCGCAGGTGCACTGGCTGGGATAAGCCCTATGTTTCAGCCCTGCG
TAGTGCCTCCAAAGCCATGGACCGGCATCACAGGGGGTGGCTATTGGGCCAACGG
TAGGCGGCCTCTGGCCCTGGTACGCACGCACAGCAAGAAGGCGCTCATGCGCTAT
GAAGACGTTTACATGCCCGAGGTTTACAAGGCGATCAATATCGCGCAGAACACCG
CCTGGAAAATCAATAAGAAGGTGTTGGCGGTCGCAAACGTGATTACCAAGTGGAA
GCATTGCCCAGTCGAGGACATACCCGCCATAGAACGCGAAGAGCTGCCGATGAAG
CCGGAAGACATTGATATGAACCCCGAGGCCCTCACCGCGTGGAAAAGAGCCGCAG
CCGCCGTATACAGGAAGGATAAAGCGCGCAAGTCCCGACGCATAAGCCTCGAGTT
TATGCTGGAACAGGCCAACAAGTTCGCCAACCACAAAGCTATCTGGTTCCCCTACA
ACATGGACTGGAGAGGGAGGGTCTACGCCGTCAGCATGTTCAATCCCCAGGGCAA
CGACATGACGAAGGGCCTTCTGACATTGGCAAAGGGGAAGCCTATCGGAAAGGAG
GGGTACTACTGGCTCAAGATCCACGGCGCCAACTGCGCGGGAGTGGACAAGGTTC
CATTTCCCGAGCGAATTAAGTTCATCGAGGAAAACCACGAAAACATTATGGCGTG
CGCTAAATCCCCCCTCGAGAACACATGGTGGGCCGAGCAAGACTCCCCGTTCTGTT
TTTTGGCATTCTGCTTTGAGTACGCCGGTGTGCAGCACCATGGCCTCTCATACAACT
GTTCCCTGCCCCTGGCCTTCGACGGAAGTTGCAGTGGGATTCAACATTTCAGCGCA
ATGTTGCGGGACGAGGTCGGTGGCAGGGCCGTTAACCTGCTCCCTTCCGAAACGGT
GCAGGACATCTACGGAATCGTGGCAAAAAAGGTAAACGAGATCCTGCAAGCGGAT
GCCATCAACGGGACGGACAATGAGGTCGTTACGGTGACAGACGAAAATACTGGGG
AAATAAGCGAAAAGGTCAAGCTGGGGACCAAAGCACTCGCGGGTCAGTGGCTCGC
CTACGGGGTGACACGCTCCGTCACCAAGAGAAGCGTGATGACCCTCGCGTACGGT
TCAAAAGAATTCGGCTTCCGCCAGCAAGTGCTGGAGGACACCATCCAGCCGGCGA
TTGACTCCGGGAAGGGTCTCATGTTTACCCAGCCGAACCAGGCCGCAGGGTACAT
GGCCAAACTGATCTGGGAAAGCGTTAGCGTCACAGTGGTCGCCGCGGTTGAGGCG
ATGAATTGGCTGAAGAGCGCGGCAAAGCTCCTCGCCGCTGAGGTGAAGGACAAAA
AGACCGGCGAAATCCTGCGCAAGCGCTGCGCCGTCCACTGGGTCACGCCGGATGG
ATTCCCCGTCTGGCAGGAGTACAAGAAGCCCATCCAAACCCGGCTCAACTTGATGT
TCCTTGGCCAGTTTCGCCTGCAGCCCACGATAAACACCAACAAAGACAGCGAGAT
CGACGCCCACAAGCAGGAGAGCGGCATCGCGCCCAACTTCGTGCACAGTCAGGAC
GGGTCCCATCTGCGGAAAACTGTTGTGTGGGCTCACGAGAAGTACGGCATTGAGA
GCTTCGCCCTGATACACGACAGCTTCGGGACCATACCAGCGGACGCAGCGAACCT
GTTCAAAGCCGTGCGGGAAACAATGGTCGACACCTACGAAAGCTGCGACGTACTG
GCAGACTTCTATGACCAATTCGCCGACCAGCTTCACGAGTCACAGCTCGACAAGAT
GCCCGCTCTGCCCGCGAAAGGCAACCTGAATTTGCGCGACATCCTTGAGAGCGATT
TTGCGTTCGCCTCTGGTGGTTCTCCCAAGAAGAAGAGGAAAGTCTAACCGGTCATC
ATCACCATCACCATTGAGTTTAAACCCGCTGATCAGCCTCGACTGTGCCTTCTAGTT
GCCAGCCATCTGTTGTTTGCCCCTCCCCCGTGCCTTCCTTGACCCTGGAAGGTGCCA
CTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGG
AAGACAATAGCAGGCATGCTGGGGATGCGGTGGGCTCTATGGCTTCTGAGGCGGA
AAGAACCAGCTGGGGCTCGATACCGTCGACCTCTAGCTAGAGCTTGGCGTAATCAT
GGTCATAGCTGTTTCCTGTGTGAAATTGTTATCCGCTCACAATTCCACACAACATA
CGAGCCGGAAGCATAAAGTGTAAAGCCTAGGGTGCCTAATGAGTGAGCTAACTCA
CATTAATTGCGTTGCGCTCACTGCCCGCTTTCCAGTCGGGAAACCTGTCGTGCCAG
CTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGTA
GGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCT
ACACTAGAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCC
TTTGATCTTTTCTACGGGGTCTGACGCTCAGTGGAACGAAAACTCACGTTAAGGGA
TTTTGGTCATGAGATTATCAAAAAGGATCTTCACCTAGATCCTTTTAAATTAAAAA
TGAAGTTTTAAATCAATCTAAAGTATATATGAGTAAACTTGGTCTGACAGTTACCA
ATGCTTAATCAGTGAGGCACCTATCTCAGCGATCTGTCTATTTCGTTCATCCATAGT
TGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTC
GTTTGGTATGGCTTCATTCAGCTCCGGTTCCCAACGATCAAGGCGAGTTACATGAT
CCCCCATGTTGTGCAAAAAAGCGGTTAGCTCCTTCGGTCCTCCGATCGTTGTCAGA
AGTAAGTTGGCCGCAGTGTTATCACTCATGGTTATGGCAGCACTGCATAATTCTCT
TACTGTCATGCCATCCGTAAGATGCTTTTCTGTGACTGGTGAGTACTCAACCAAGT
CATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGTAAC
CCACTCGTGCACCCAACTGATCTTCAGCATCTTTTACTTTCACCAGCGTTTCTGGGT
GAGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGTT
ATTGTCTCATGAGCGGATACATATTTGAATGTATTTAGAAAAATAAACAAATAGGG
GTTCCGCGCACATTTCCCCGAAAAGTGCCACCTGACGTCGACGGATCGGGAGATC
GATCTCCCGATCCCCTAGGGTCGACTCTCAGTACAATCTGCTCTGATGCCGCATAG
TTAAGCCAGTATCTGCTCCCTGCTTGTGTGTTGGAGGTCGCTGAGTAGTGCGCGAG
CAAAATTTAAGCTACAACAAGGCAAGGCTTGACCGACAATTGCATGAAGAATCTG
CTTAGGGTTAGGCGTTTTGCGCTGCTTCGCGATGTACGGGCCAGATATACGCGTTG
ACATTGATTATTGACTAGTTATTAATAGTAATCAATTACGGGGTCATTAGTTCATA
GCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCC
ACTTGGCAGTACATCAAGTGTATC
AID*Δ-T7 RNA Polymerase-NLS polypeptide sequence (SEQ ID NO: 32):
MDSLLMNRREFLYQFKNVRWAKGRRETYLCYVVKRRDSATSFSLDFGYLRNKNGCH
VELLFLRYISDWDLDPGRCYRVTWFISWSPCYDCARHVADFLRGNPNLSLRIFTARLY
FCEDRKAEPEGLRRLHRAGVQIAIMTFKDYFYCWNTFVENHGRTFKAWEGLHENSVR
LSRQLRRILLPLYEVDDLRDAFRTSGSETPGTSESATPESNTINIAKNDFSDIELAAIPFNT
LADHYGERLAREQLALEHESYEMGEARFRKMFERQLKAGEVADNAAAKPLITTLLPK
MIARINDWFEEVKAKRGKRPTAFQFLQEIKPEAVAYITIKTTLACLTSADNTTVQAVAS
AIGRAIEDEARFGRIRDLEAKHFKKNVEEQLNKRVGHVYKKAFMQVVEADMLSKGLL
GGEAWSSWHKEDSIHVGVRCIEMLIESTGMVSLHRQNAGVVGQDSETIELAPEYAEAI
ATRAGALAGISPMFQPCVVPPKPWTGITGGGYWANGRRPLALVRTHSKKALMRYED
VYMPEVYKAINIAQNTAWKINKKVLAVANVITKWKHCPVEDIPAIEREELPMKPEDID
MNPEALTAWKRAAAAVYRKDKARKSRRISLEFMLEQANKFANHKAIWFPYNMDWR
GRVYAVSMFNPQGNDMTKGLLTLAKGKPIGKEGYYWLKIHGANCAGVDKVPFPERI
KFIEENHENIMACAKSPLENTWWAEQDSPFCFLAFCFEYAGVQHHGLSYNCSLPLAFD
GSCSGIQHFSAMLRDEVGGRAVNLLPSETVQDIYGIVAKKVNEILQADAINGTDNEVV
TVTDENTGEISEKVKLGTKALAGQWLAYGVTRSVTKRSVMTLAYGSKEFGFRQQVLE
DTIQPAIDSGKGLMFTQPNQAAGYMAKLIWESVSVTVVAAVEAMNWLKSAAKLLAA
EVKDKKTGEILRKRCAVHWVTPDGFPVWQEYKKPIQTRLNLMFLGQFRLQPTINTNK
DSEIDAHKQESGIAPNFVHSQDGSHLRKTVVWAHEKYGIESFALIHDSFGTIPADAANL
FKAVRETMVDTYESCDVLADFYDQFADQLHESQLDKMPALPAKGNLNLRDILESDFA
FASGGSPKKKRKV
Hyperactive AID*Δ-T7 RNA Polymerase Uracil DNA Glycosylase
Inhibitor (UGI)-NLS plasmid DNA sequence (SEQ ID NO: 33):
ATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTA
GTCATCGCTATTACCATGGTGATGCGGTTTTGGCAGTACATCAATGGGCGTGGATA
GCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCA
TTGACGCAAATGGGCGGTAGGCGTGTACGGTGGGAGGTCTATATAAGCAGAGCTG
GTTTAGTGAACCGTCAGATCCGCTAGAGATCCGCGGCCGCGAGAGCCGCCACCAT
GGACAGCCTCTTGATGAACCGGAGGGAGTTTCTTTACCAATTCAAAAATGTCCGCT
GGGCTAAGGGTCGGCGTGAGACCTACCTGTGCTACGTAGTGAAGAGGCGTGACAG
TGCTACATCCTTTTCACTGGACTTTGGTTATCTTCGCAATAAGAACGGCTGCCACGT
GGAATTGCTCTTCCTCCGCTACATCTCGGACTGGGACCTAGACCCTGGCCGCTGCT
ACCGCGTCACCTGGTTCATCTCCTGGAGCCCCTGCTACGACTGTGCCCGACATGTG
GCCGACTTTCTGCGAGGGAACCCCAACCTCAGTCTGAGGATCTTCACCGCGCGCCT
CTACTTCTGTGAGGACCGCAAGGCTGAGCCCGAGGGGCTGCGGCGGCTGCACCGC
GCCGGGGTGCAAATAGCCATCATGACCTTCAAAGATTATTTTTACTGCTGGAATAC
TTTTGTAGAAAACCACGGAAGAACTTTCAAAGCCTGGGAAGGGCTGCATGAAAAT
TCAGTTCGTCTCTCCAGACAGCTTCGGCGCATCCTTTTGCCCCTGTATGAGGTTGAT
GACTTACGAGACGCATTTCGTACTAGCGGCAGCGAGACTCCCGGGACCTCAGAGT
CCGCCACACCCGAAAGTAACACCATCAACATTGCTAAGAACGACTTCTCAGACAT
AGAGCTCGCGGCTATTCCGTTCAACACCCTGGCTGACCACTACGGCGAGAGACTCG
CTAGGGAGCAGCTGGCGTTGGAGCATGAATCCTACGAGATGGGCGAGGCTAGGTT
CCGCAAGATGTTCGAGCGACAATTGAAGGCAGGGGAGGTGGCGGACAACGCTGCC
GCCAAGCCCCTGATCACAACCTTGCTGCCCAAAATGATCGCGCGGATCAACGATTG
GTTTGAGGAGGTTAAGGCAAAACGGGGCAAACGCCCGACCGCATTTCAATTCCTC
CAAGAAATCAAGCCTGAGGCTGTTGCCTACATCACTATCAAGACGACACTGGCGT
GTCTCACAAGCGCCGACAACACCACCGTGCAAGCCGTCGCCAGCGCCATCGGGCG
GGCAATTGAGGATGAGGCACGGTTTGGTAGGATCCGAGACCTGGAAGCGAAGCAC
TTCAAGAAGAACGTGGAAGAGCAGTTGAACAAACGCGTCGGCCACGTGTATAAAA
AGGCTTTCATGCAGGTGGTGGAGGCCGATATGCTCAGTAAGGGGCTGCTTGGGGG
GGAGGCGTGGTCATCCTGGCACAAGGAGGATAGCATTCACGTGGGGGTCCGATGT
ATCGAGATGCTGATAGAGAGCACCGGAATGGTCTCCCTCCATCGCCAGAACGCTG
GGGTCGTAGGGCAGGACTCCGAGACTATTGAGCTGGCCCCCGAGTATGCCGAAGC
AATCGCTACACGCGCAGGTGCACTGGCTGGGATAAGCCCTATGTTTCAGCCCTGCG
TAGTGCCTCCAAAGCCATGGACCGGCATCACAGGGGGTGGCTATTGGGCCAACGG
TAGGCGGCCTCTGGCCCTGGTACGCACGCACAGCAAGAAGGCGCTCATGCGCTAT
GAAGACGTTTACATGCCCGAGGTTTACAAGGCGATCAATATCGCGCAGAACACCG
CCTGGAAAATCAATAAGAAGGTGTTGGCGGTCGCAAACGTGATTACCAAGTGGAA
GCATTGCCCAGTCGAGGACATACCCGCCATAGAACGCGAAGAGCTGCCGATGAAG
CCGGAAGACATTGATATGAACCCCGAGGCCCTCACCGCGTGGAAAAGAGCCGCAG
CCGCCGTATACAGGAAGGATAAAGCGCGCAAGTCCCGACGCATAAGCCTCGAGTT
TATGCTGGAACAGGCCAACAAGTTCGCCAACCACAAAGCTATCTGGTTCCCCTACA
ACATGGACTGGAGAGGGAGGGTCTACGCCGTCAGCATGTTCAATCCCCAGGGCAA
CGACATGACGAAGGGCCTTCTGACATTGGCAAAGGGGAAGCCTATCGGAAAGGAG
GGGTACTACTGGCTCAAGATCCACGGCGCCAACTGCGCGGGAGTGGACAAGGTTC
CATTTCCCGAGCGAATTAAGTTCATCGAGGAAAACCACGAAAACATTATGGCGTG
CGCTAAATCCCCCCTCGAGAACACATGGTGGGCCGAGCAAGACTCCCCGTTCTGTT
TTTTGGCATTCTGCTTTGAGTACGCCGGTGTGCAGCACCATGGCCTCTCATACAACT
GTTCCCTGCCCCTGGCCTTCGACGGAAGTTGCAGTGGGATTCAACATTTCAGCGCA
ATGTTGCGGGACGAGGTCGGTGGCAGGGCCGTTAACCTGCTCCCTTCCGAAACGGT
GCAGGACATCTACGGAATCGTGGCAAAAAAGGTAAACGAGATCCTGCAAGCGGAT
GCCATCAACGGGACGGACAATGAGGTCGTTACGGTGACAGACGAAAATACTGGGG
AAATAAGCGAAAAGGTCAAGCTGGGGACCAAAGCACTCGCGGGTCAGTGGCTCGC
CTACGGGGTGACACGCTCCGTCACCAAGAGAAGCGTGATGACCCTCGCGTACGGT
TCAAAAGAATTCGGCTTCCGCCAGCAAGTGCTGGAGGACACCATCCAGCCGGCGA
TTGACTCCGGGAAGGGTCTCATGTTTACCCAGCCGAACCAGGCCGCAGGGTACAT
GGCCAAACTGATCTGGGAAAGCGTTAGCGTCACAGTGGTCGCCGCGGTTGAGGCG
ATGAATTGGCTGAAGAGCGCGGCAAAGCTCCTCGCCGCTGAGGTGAAGGACAAAA
AGACCGGCGAAATCCTGCGCAAGCGCTGCGCCGTCCACTGGGTCACGCCGGATGG
ATTCCCCGTCTGGCAGGAGTACAAGAAGCCCATCCAAACCCGGCTCAACTTGATGT
TCCTTGGCCAGTTTCGCCTGCAGCCCACGATAAACACCAACAAAGACAGCGAGAT
CGACGCCCACAAGCAGGAGAGCGGCATCGCGCCCAACTTCGTGCACAGTCAGGAC
GGGTCCCATCTGCGGAAAACTGTTGTGTGGGCTCACGAGAAGTACGGCATTGAGA
GCTTCGCCCTGATACACGACAGCTTCGGGACCATACCAGCGGACGCAGCGAACCT
GTTCAAAGCCGTGCGGGAAACAATGGTCGACACCTACGAAAGCTGCGACGTACTG
GCAGACTTCTATGACCAATTCGCCGACCAGCTTCACGAGTCACAGCTCGACAAGAT
GCCCGCTCTGCCCGCGAAAGGCAACCTGAATTTGCGCGACATCCTTGAGAGCGATT
TTGCGTTCGCCTCTGGTGGTTCTCCCAAGAAGAAGAGGAAAGTCTAACCGGTCATC
ATCACCATCACCATTGAGTTTAAACCCGCTGATCAGCCTCGACTGTGCCTTCTAGTT
GCCAGCCATCTGTTGTTTGCCCCTCCCCCGTGCCTTCCTTGACCCTGGAAGGTGCCA
CTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGG
AAGACAATAGCAGGCATGCTGGGGATGCGGTGGGCTCTATGGCTTCTGAGGCGGA
AAGAACCAGCTGGGGCTCGATACCGTCGACCTCTAGCTAGAGCTTGGCGTAATCAT
GGTCATAGCTGTTTCCTGTGTGAAATTGTTATCCGCTCACAATTCCACACAACATA
CGAGCCGGAAGCATAAAGTGTAAAGCCTAGGGTGCCTAATGAGTGAGCTAACTCA
CATTAATTGCGTTGCGCTCACTGCCCGCTTTCCAGTCGGGAAACCTGTCGTGCCAG
CTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGTA
GGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCT
ACACTAGAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCC
TTTGATCTTTTCTACGGGGTCTGACGCTCAGTGGAACGAAAACTCACGTTAAGGGA
TTTTGGTCATGAGATTATCAAAAAGGATCTTCACCTAGATCCTTTTAAATTAAAAA
TGAAGTTTTAAATCAATCTAAAGTATATATGAGTAAACTTGGTCTGACAGTTACCA
ATGCTTAATCAGTGAGGCACCTATCTCAGCGATCTGTCTATTTCGTTCATCCATAGT
TGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTC
GTTTGGTATGGCTTCATTCAGCTCCGGTTCCCAACGATCAAGGCGAGTTACATGAT
CCCCCATGTTGTGCAAAAAAGCGGTTAGCTCCTTCGGTCCTCCGATCGTTGTCAGA
AGTAAGTTGGCCGCAGTGTTATCACTCATGGTTATGGCAGCACTGCATAATTCTCT
TACTGTCATGCCATCCGTAAGATGCTTTTCTGTGACTGGTGAGTACTCAACCAAGT
CATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGTAAC
CCACTCGTGCACCCAACTGATCTTCAGCATCTTTTACTTTCACCAGCGTTTCTGGGT
GAGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGTT
ATTGTCTCATGAGCGGATACATATTTGAATGTATTTAGAAAAATAAACAAATAGGG
GTTCCGCGCACATTTCCCCGAAAAGTGCCACCTGACGTCGACGGATCGGGAGATC
GATCTCCCGATCCCCTAGGGTCGACTCTCAGTACAATCTGCTCTGATGCCGCATAG
TTAAGCCAGTATCTGCTCCCTGCTTGTGTGTTGGAGGTCGCTGAGTAGTGCGCGAG
CAAAATTTAAGCTACAACAAGGCAAGGCTTGACCGACAATTGCATGAAGAATCTG
CTTAGGGTTAGGCGTTTTGCGCTGCTTCGCGATGTACGGGCCAGATATACGCGTTG
ACATTGATTATTGACTAGTTATTAATAGTAATCAATTACGGGGTCATTAGTTCATA
GCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCC
ACTTGGCAGTACATCAAGTGTATC
AID*Δ-T7 RNA Polymerase-UGI-NLS polypeptide sequence
(SEQ ID NO: 34):
MDSLLMNRREFLYQFKNVRWAKGRRETYLCYVVKRRDSATSFSLDFGYLRNKNGCH
VELLFLRYISDWDLDPGRCYRVTWFISWSPCYDCARHVADFLRGNPNLSLRIFTARLY
FCEDRKAEPEGLRRLHRAGVQIAIMTFKDYFYCWNTFVENHGRTFKAWEGLHENSVR
LSRQLRRILLPLYEVDDLRDAFRTSGSETPGTSESATPESNTINIAKNDFSDIELAAIPFNT
LADHYGERLAREQLALEHESYEMGEARFRKMFERQLKAGEVADNAAAKPLITTLLPK
MIARINDWFEEVKAKRGKRPTAFQFLQEIKPEAVAYITIKTTLACLTSADNTTVQAVAS
AIGRAIEDEARFGRIRDLEAKHFKKNVEEQLNKRVGHVYKKAFMQVVEADMLSKGLL
GGEAWSSWHKEDSIHVGVRCIEMLIESTGMVSLHRQNAGVVGQDSETIELAPEYAEAI
ATRAGALAGISPMFQPCVVPPKPWTGITGGGYWANGRRPLALVRTHSKKALMRYED
VYMPEVYKAINIAQNTAWKINKKVLAVANVITKWKHCPVEDIPAIEREELPMKPEDID
MNPEALTAWKRAAAAVYRKDKARKSRRISLEFMLEQANKFANHKAIWFPYNMDWR
GRVYAVSMFNPQGNDMTKGLLTLAKGKPIGKEGYYWLKIHGANCAGVDKVPFPERI
KFIEENHENIMACAKSPLENTWWAEQDSPFCFLAFCFEYAGVQHHGLSYNCSLPLAFD
GSCSGIQHFSAMLRDEVGGRAVNLLPSETVQDIYGIVAKKVNEILQADAINGTDNEVV
TVTDENTGEISEKVKLGTKALAGQWLAYGVTRSVTKRSVMTLAYGSKEFGFRQQVLE
DTIQPAIDSGKGLMFTQPNQAAGYMAKLIWESVSVTVVAAVEAMNWLKSAAKLLAA
EVKDKKTGEILRKRCAVHWVTPDGFPVWQEYKKPIQTRLNLMFLGQFRLQPTINTNK
DSEIDAHKQESGIAPNFVHSQDGSHLRKTVVWAHEKYGIESFALIHDSFGTIPADAANL
FKAVRETMVDTYESCDVLADFYDQFADQLHESQLDKMPALPAKGNLNLRDILESDFA
FASGGSTNLSDIIEKETGKQLVIQESILMLPEEVEEVIGNKPESDILVHTAYDESTDENV
MLLTSDAPEYKPWALVIQDSNGENKIKMLSGGSPKKKRKV
ecTadA DNA sequence (SEQ ID NO: 35):
ATGTCCGAAGTCGAGTTTTCCCATGAGTACTGGATGAGACACGCATTGACTCTCGC
AAAGAGGGCTTGGGATGAACGCGAGGTGCCCGTGGGGGCAGTACTCGTGCATAAC
AATCGCGTAATCGGCGAAGGTTGGAATAGGCCGATCGGACGCCACGACCCCACTG
CACATGCGGAAATCATGGCCCTTCGACAGGGAGGGCTTGTGATGCAGAATTATCG
ACTTATCGATGCGACGCTGTACGTCACGCTTGAACCTTGCGTAATGTGCGCGGGAG
CTATGATTCACTCCCGCATTGGACGAGTTGTATTCGGTGCCCGCGACGCCAAGACG
GGTGCCGCAGGTTCACTGATGGACGTGCTGCATCACCCAGGCATGAACCACCGGG
TAGAAATCACAGAAGGCATATTGGCGGACGAATGTGCGGCGCTGTTGTCCGACTTT
TTTCGCATGCGGAGGCAGGAGATCAAGGCCCAGAAAAAAGCACAATCCTCTACTG
AC
ecTadA polypeptide sequence (SEQ ID NO: 36):
MSEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARDAKTGA
AGSLMDVLHHPGMNHRVEITEGILADECAALLSDFFRMRRQEIKAQKKAQSSTD
Rattus norvegicus APOBEC1 DNA sequence (SEQ ID NO: 37):
ATGAGCTCAGAGACTGGCCCAGTGGCTGTGGACCCCACATTGAGACGGCGGATCG
AGCCCCATGAGTTTGAGGTATTCTTCGATCCGAGAGAGCTCCGCAAGGAGACCTGC
CTGCTTTACGAAATTAATTGGGGGGGCCGGCACTCCATTTGGCGACATACATCACA
GAACACTAACAAGCACGTCGAAGTCAACTTCATCGAGAAGTTCACGACAGAAAGA
TATTTCTGTCCGAACACAAGGTGCAGCATTACCTGGTTTCTCAGCTGGAGCCCATG
CGGCGAATGTAGTAGGGCCATCACTGAATTCCTGTCAAGGTATCCCCACGTCACTC
TGTTTATTTACATCGCAAGGCTGTACCACCACGCTGACCCCCGCAATCGACAAGGC
CTGCGGGATTTGATCTCTTCAGGTGTGACTATCCAAATTATGACTGAGCAGGAGTC
AGGATACTGCTGGAGAAACTTTGTGAATTATAGCCCGAGTAATGAAGCCCACTGG
CCTAGGTATCCCCATCTGTGGGTACGACTGTACGTTCTTGAACTGTACTGCATCAT
ACTGGGCCTGCCTCCTTGTCTCAACATTCTGAGAAGGAAGCAGCCACAGCTGACAT
TCTTTACCATCGCTCTTCAGTCTTGTCATTACCAGCGACTGCCCCCACACATTCTCT
GGGCCACCGGGTTGAAA
SP6 RNA Polymerase DNA sequence (SEQ ID NO: 38):
CAAGATTTACACGCTATCCAGCTTCAATTAGAAGAAGAGATGTTTAATGGTGGCAT
TCGTCGCTTCGAAGCAGATCAACAACGCCAGATTGCAGCAGGTAGCGAGAGCGAC
ACAGCATGGAACCGCCGCCTGTTGTCAGAACTTATTGCACCTATGGCTGAAGGCAT
TCAGGCTTATAAAGAAGAGTACGAAGGTAAGAAAGGTCGTGCACCTCGCGCATTG
GCTTTCTTACAATGTGTAGAAAATGAAGTTGCAGCATACATCACTATGAAAGTTGT
TATGGATATGCTGAATACGGATGCTACCCTTCAGGCTATTGCAATGAGTGTAGCAG
AACGCATTGAAGACCAAGTGCGCTTTTCTAAGCTAGAAGGTCACGCCGCTAAATA
CTTTGAGAAGGTTAAGAAGTCACTCAAGGCTAGCCGTACTAAGTCATATCGTCACG
CTCATAACGTAGCTGTAGTTGCTGAAAAATCAGTTGCAGAAAAGGACGCGGACTT
TGACCGTTGGGAGGCGTGGCCAAAAGAAACTCAATTGCAGATTGGTACTACCTTG
CTTGAAATCTTAGAAGGTAGCGTTTTCTATAATGGTGAACCTGTATTTATGCGTGCT
ATGCGCACTTATGGCGGAAAGACTATTTACTACTTACAAACTTCTGAAAGTGTAGG
CCAGTGGATTAGCGCATTCAAAGAGCACGTAGCGCAATTAAGCCCAGCTTATGCC
CCTTGCGTAATCCCTCCTCGTCCTTGGAGAACTCCATTTAATGGAGGGTTCCATACT
GAGAAGGTAGCTAGCCGTATCCGTCTTGTAAAAGGTAACCGTGAGCATGTACGCA
AGTTGACTCAAAAGCAAATGCCAAAGGTTTATAAGGCTATCAACGCATTACAAAA
TACACAATGGCAAATCAACAAGGATGTATTAGCAGTTATTGAAGAAGTAATCCGC
TTAGACCTTGGTTATGGTGTACCTTCCTTCAAGCCACTGATTGACAAGGAGAACAA
GCCAGCTAACCCGGTACCTGTTGAATTCCAACACCTGCGCGGTCGTGAACTGAAAG
AGATGCTATCACCTGAGCAGTGGCAACAATTCATTAACTGGAAAGGCGAATGCGC
GCGCCTATATACCGCAGAAACTAAGCGCGGTTCAAAGTCCGCCGCCGTTGTTCGCA
TGGTAGGACAGGCCCGTAAATATAGCGCCTTTGAATCCATTTACTTCGTGTACGCA
ATGGATAGCCGCAGCCGTGTCTATGTGCAATCTAGCACGCTCTCTCCGCAGTCTAA
CGACTTAGGTAAGGCATTACTCCGCTTTACCGAGGGACGCCCTGTGAATGGCGTAG
AAGCGCTTAAATGGTTCTGCATCAATGGTGCTAACCTTTGGGGATGGGACAAGAA
AACTTTTGATGTGCGCGTGTCTAACGTATTAGATGAGGAATTCCAAGATATGTGTC
GAGACATCGCCGCAGACCCTCTCACATTCACCCAATGGGCTAAAGCTGATGCACCT
TATGAATTCCTCGCTTGGTGCTTTGAGTATGCTCAATACCTTGATTTGGTGGATGAA
GGAAGGGCCGACGAATTCCGCACTCACCTACCAGTACATCAGGACGGGTCTTGTTC
AGGCATTCAGCACTATAGTGCTATGCTTCGCGACGAAGTAGGGGCCAAAGCTGTT
AACCTGAAACCCTCCGATGCACCGCAGGATATCTATGGGGCGGTGGCGCAAGTGG
TTATCAAGAAGAATGCGCTATATATGGATGCGGACGATGCAACCACGTTTACTTCT
GGTAGCGTCACGCTGTCCGGTACAGAACTGCGAGCAATGGCTAGCGCATGGGATA
GTATTGGTATTACCCGTAGCTTAACCAAAAAGCCCGTGATGACCTTGCCATATGGT
TCTACTCGCTTAACTTGCCGTGAATCTGTGATTGATTACATCGTAGACTTAGAGGA
AAAAGAGGCGCAGAAGGCAGTAGCAGAAGGGCGGACGGCAAACAAGGTACATCC
TTTTGAAGACGATCGTCAAGATTACTTGACTCCGGGCGCAGCTTACAACTACATGA
CGGCACTAATCTGGCCTTCTATTTCTGAAGTAGTTAAGGCACCGATAGTAGCTATG
AAGATGATACGCCAGCTTGCACGCTTTGCAGCGAAACGTAATGAAGGCCTGATGT
ACACCCTGCCTACTGGCTTCATCTTAGAACAGAAGATCATGGCAACCGAGATGCTA
CGCGTGCGTACCTGTCTGATGGGTGATATCAAGATGTCCCTTCAGGTTGAAACGGA
TATCGTAGATGAAGCCGCTATGATGGGAGCAGCAGCACCTAATTTCGTACACGGTC
ATGACGCAAGTCACCTTATCCTTACCGTATGTGAATTGGTAGACAAGGGCGTAACT
AGTATCGCTGTAATCCACGACTCTTTTGGTACTCATGCAGACAACACCCTCACTCTT
AGAGTGGCACTTAAAGGGCAGATGGTTGCAATGTATATTGATGGTAATGCGCTTCA
GAAACTACTGGAGGAGCATGAAGAGCGCTGGATGGTTGATACAGGTATCGAAGTA
CCTGAGCAAGGGGAGTTCGACCTTAACGAAATCATGGATTCTGAATACGTATTTGC
C
SP6 RNA Polymerase polypeptide sequence (SEQ ID NO: 39):
QDLHAIQLQLEEEMFNGGIRRFEADQQRQIAAGSESDTAWNRRLLSELIAPMAEGIQA
YKEEYEGKKGRAPRALAFLQCVENEVAAYITMKVVMDMLNTDATLQAIAMSVAERI
EDQVRFSKLEGHAAKYFEKVKKSLKASRTKSYRHAHNVAVVAEKSVAEKDADFDRW
EAWPKETQLQIGTTLLEILEGSVFYNGEPVFMRAMRTYGGKTIYYLQTSESVGQWISA
FKEHVAQLSPAYAPCVIPPRPWRTPFNGGFHTEKVASRIRLVKGNREHVRKLTQKQMP
KVYKAINALQNTQWQINKDVLAVIEEVIRLDLGYGVPSFKPLIDKENKPANPVPVEFQ
HLRGRELKEMLSPEQWQQFINWKGECARLYTAETKRGSKSAAVVRMVGQARKYSAF
ESIYFVYAMDSRSRVYVQSSTLSPQSNDLGKALLRFTEGRPVNGVEALKWFCINGANL
WGWDKKTFDVRVSNVLDEEFQDMCRDIAADPLTFTQWAKADAPYEFLAWCFEYAQ
YLDLVDEGRADEFRTHLPVHQDGSCSGIQHYSAMLRDEVGAKAVNLKPSDAPQDIYG
AVAQVVIKKNALYMDADDATTFTSGSVTLSGTELRAMASAWDSIGITRSLTKKPVMT
LPYGSTRLTCRESVIDYIVDLEEKEAQKAVAEGRTANKVHPFEDDRQDYLTPGAAYNY
MTALIWPSISEVVKAPIVAMKMIRQLARFAAKRNEGLMYTLPTGFILEQKIMATEMLR
VRTCLMGDIKMSLQVETDIVDEAAMMGAAAPNFVHGHDASHLILTVCELVDKGVTSI
AVIHDSFGTHADNTLTLRVALKGQMVAMYIDGNALQKLLEEHEERWMVDTGIEVPEQ
GEFDLNEIMDSEYVFA
SV40 nuclear localization signal (NLS) DNA sequence
(SEQ ID NO: 40):
CCCAAGAAGAAGAGGAAAGTC
SV40 NLS polypeptide sequence (SEQ ID NO: 41):
PKKKRKV
T7 RNA Polymerase DNA sequence (SEQ ID NO: 42):
ATGAACACCATCAACATTGCTAAGAACGACTTCTCAGACATAGAGCTCGCGGCTAT
TCCGTTCAACACCCTGGCTGACCACTACGGCGAGAGACTCGCTAGGGAGCAGCTG
GCGTTGGAGCATGAATCCTACGAGATGGGCGAGGCTAGGTTCCGCAAGATGTTCG
AGCGACAATTGAAGGCAGGGGAGGTGGCGGACAACGCTGCCGCCAAGCCCCTGAT
CACAACCTTGCTGCCCAAAATGATCGCGCGGATCAACGATTGGTTTGAGGAGGTTA
AGGCAAAACGGGGCAAACGCCCGACCGCATTTCAATTCCTCCAAGAAATCAAGCC
TGAGGCTGTTGCCTACATCACTATCAAGACGACACTGGCGTGTCTCACAAGCGCCG
ACAACACCACCGTGCAAGCCGTCGCCAGCGCCATCGGGCGGGCAATTGAGGATGA
GGCACGGTTTGGTAGGATCCGAGACCTGGAAGCGAAGCACTTCAAGAAGAACGTG
GAAGAGCAGTTGAACAAACGCGTCGGCCACGTGTATAAAAAGGCTTTCATGCAGG
TGGTGGAGGCCGATATGCTCAGTAAGGGGCTGCTTGGGGGGGAGGCGTGGTCATC
CTGGCACAAGGAGGATAGCATTCACGTGGGGGTCCGATGTATCGAGATGCTGATA
GAGAGCACCGGAATGGTCTCCCTCCATCGCCAGAACGCTGGGGTCGTAGGGCAGG
ACTCCGAGACTATTGAGCTGGCCCCCGAGTATGCCGAAGCAATCGCTACACGCGC
AGGTGCACTGGCTGGGATAAGCCCTATGTTTCAGCCCTGCGTAGTGCCTCCAAAGC
CATGGACCGGCATCACAGGGGGTGGCTATTGGGCCAACGGTAGGCGGCCTCTGGC
CCTGGTACGCACGCACAGCAAGAAGGCGCTCATGCGCTATGAAGACGTTTACATG
CCCGAGGTTTACAAGGCGATCAATATCGCGCAGAACACCGCCTGGAAAATCAATA
AGAAGGTGTTGGCGGTCGCAAACGTGATTACCAAGTGGAAGCATTGCCCAGTCGA
GGACATACCCGCCATAGAACGCGAAGAGCTGCCGATGAAGCCGGAAGACATTGAT
ATGAACCCCGAGGCCCTCACCGCGTGGAAAAGAGCCGCAGCCGCCGTATACAGGA
AGGATAAAGCGCGCAAGTCCCGACGCATAAGCCTCGAGTTTATGCTGGAACAGGC
CAACAAGTTCGCCAACCACAAAGCTATCTGGTTCCCCTACAACATGGACTGGAGA
GGGAGGGTCTACGCCGTCAGCATGTTCAATCCCCAGGGCAACGACATGACGAAGG
GCCTTCTGACATTGGCAAAGGGGAAGCCTATCGGAAAGGAGGGGTACTACTGGCT
CAAGATCCACGGCGCCAACTGCGCGGGAGTGGACAAGGTTCCATTTCCCGAGCGA
ATTAAGTTCATCGAGGAAAACCACGAAAACATTATGGCGTGCGCTAAATCCCCCCT
CGAGAACACATGGTGGGCCGAGCAAGACTCCCCGTTCTGTTTTTTGGCATTCTGCT
TTGAGTACGCCGGTGTGCAGCACCATGGCCTCTCATACAACTGTTCCCTGCCCCTG
GCCTTCGACGGAAGTTGCAGTGGGATTCAACATTTCAGCGCAATGTTGCGGGACG
AGGTCGGTGGCAGGGCCGTTAACCTGCTCCCTTCCGAAACGGTGCAGGACATCTAC
GGAATCGTGGCAAAAAAGGTAAACGAGATCCTGCAAGCGGATGCCATCAACGGG
ACGGACAATGAGGTCGTTACGGTGACAGACGAAAATACTGGGGAAATAAGCGAA
AAGGTCAAGCTGGGGACCAAAGCACTCGCGGGTCAGTGGCTCGCCTACGGGGTGA
CACGCTCCGTCACCAAGAGAAGCGTGATGACCCTCGCGTACGGTTCAAAAGAATT
CGGCTTCCGCCAGCAAGTGCTGGAGGACACCATCCAGCCGGCGATTGACTCCGGG
AAGGGTCTCATGTTTACCCAGCCGAACCAGGCCGCAGGGTACATGGCCAAACTGA
TCTGGGAAAGCGTTAGCGTCACAGTGGTCGCCGCGGTTGAGGCGATGAATTGGCT
GAAGAGCGCGGCAAAGCTCCTCGCCGCTGAGGTGAAGGACAAAAAGACCGGCGA
AATCCTGCGCAAGCGCTGCGCCGTCCACTGGGTCACGCCGGATGGATTCCCCGTCT
GGCAGGAGTACAAGAAGCCCATCCAAACCCGGCTCAACTTGATGTTCCTTGGCCA
GTTTCGCCTGCAGCCCACGATAAACACCAACAAAGACAGCGAGATCGACGCCCAC
AAGCAGGAGAGCGGCATCGCGCCCAACTTCGTGCACAGTCAGGACGGGTCCCATC
TGCGGAAAACTGTTGTGTGGGCTCACGAGAAGTACGGCATTGAGAGCTTCGCCCT
GATACACGACAGCTTCGGGACCATACCAGCGGACGCAGCGAACCTGTTCAAAGCC
GTGCGGGAAACAATGGTCGACACCTACGAAAGCTGCGACGTACTGGCAGACTTCT
ATGACCAATTCGCCGACCAGCTTCACGAGTCACAGCTCGACAAGATGCCCGCTCTG
CCCGCGAAAGGCAACCTGAATTTGCGCGACATCCTTGAGAGCGATTTTGCGTTCGC
C
T7 RNA Polymerase polypeptide sequence (SEQ ID NO: 43):
MNTINIAKNDFSDIELAAIPFNTLADHYGERLAREQLALEHESYEMGEARFRKMFERQ
LKAGEVADNAAAKPLITTLLPKMIARINDWFEEVKAKRGKRPTAFQFLQEIKPEAVAYI
TIKTTLACLTSADNTTVQAVASAIGRAIEDEARFGRIRDLEAKHFKKNVEEQLNKRVG
HVYKKAFMQVVEADMLSKGLLGGEAWSSWHKEDSIHVGVRCIEMLIESTGMVSLHR
QNAGVVGQDSETIELAPEYAEAIATRAGALAGISPMFQPCVVPPKPWTGITGGGYWAN
GRRPLALVRTHSKKALMRYEDVYMPEVYKAINIAQNTAWKINKKVLAVANVITKWK
HCPVEDIPAIEREELPMKPEDIDMNPEALTAWKRAAAAVYRKDKARKSRRISLEFMLE
QANKFANHKAIWFPYNMDWRGRVYAVSMFNPQGNDMTKGLLTLAKGKPIGKEGYY
WLKIHGANCAGVDKVPFPERIKFIEENHENIMACAKSPLENTWWAEQDSPFCFLAFCF
EYAGVQHHGLSYNCSLPLAFDGSCSGIQHFSAMLRDEVGGRAVNLLPSETVQDIYGIV
AKKVNEILQADAINGTDNEVVTVTDENTGEISEKVKLGTKALAGQWLAYGVTRSVTK
RSVMTLAYGSKEFGFRQQVLEDTIQPAIDSGKGLMFTQPNQAAGYMAKLIWESVSVT
VVAAVEAMNWLKSAAKLLAAEVKDKKTGEILRKRCAVHWVTPDGFPVWQEYKKPI
QTRLNLMFLGQFRLQPTINTNKDSEIDAHKQESGIAPNFVHSQDGSHLRKTVVWAHEK
YGIESFALIHDSFGTIPADAANLFKAVRETMVDTYESCDVLADFYDQFADQLHESQLD
KMPALPAKGNLNLRDILESDFAFA
Uracil DNA Glycosylase Inhibitor (UGI) DNA sequence
(SEQ ID NO: 44):
ACTAATCTGTCAGATATTATTGAAAAGGAGACCGGTAAGCAACTGGTTATCCAGG
AATCCATCCTCATGCTCCCAGAGGAGGTGGAAGAAGTCATTGGGAACAAGCCGGA
AAGCGATATACTCGTGCACACCGCCTACGACGAGAGCACCGACGAGAATGTCATG
CTTCTGACTAGCGACGCCCCTGAATACAAGCCTTGGGCTCTGGTCATACAGGATAG
CAACGGTGAGAACAAGATTAAGATGCTC
UGI polypeptide sequence (SEQ ID NO: 45):
TNLSDIIEKETGKQLVIQESILMLPEEVEEVIGNKPESDILVHTAYDESTDENVMLLTSD
APEYKPWALVIQDSNGENKIKML
Rattus norvegicus APOBEC1-T7 Polymerase-NLS plasmid DNA
sequence (SEQ ID NO: 46):
ATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTA
GTCATCGCTATTACCATGGTGATGCGGTTTTGGCAGTACATCAATGGGCGTGGATA
GCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCA
TTGACGCAAATGGGCGGTAGGCGTGTACGGTGGGAGGTCTATATAAGCAGAGCTG
GTTTAGTGAACCGTCAGATCCGCTAGAGATCCGCGGCCGCGAGAGCCGCCACCAT
GAGCTCAGAGACTGGCCCAGTGGCTGTGGACCCCACATTGAGACGGCGGATCGAG
CCCCATGAGTTTGAGGTATTCTTCGATCCGAGAGAGCTCCGCAAGGAGACCTGCCT
GCTTTACGAAATTAATTGGGGGGGCCGGCACTCCATTTGGCGACATACATCACAGA
ACACTAACAAGCACGTCGAAGTCAACTTCATCGAGAAGTTCACGACAGAAAGATA
TTTCTGTCCGAACACAAGGTGCAGCATTACCTGGTTTCTCAGCTGGAGCCCATGCG
GCGAATGTAGTAGGGCCATCACTGAATTCCTGTCAAGGTATCCCCACGTCACTCTG
TTTATTTACATCGCAAGGCTGTACCACCACGCTGACCCCCGCAATCGACAAGGCCT
GCGGGATTTGATCTCTTCAGGTGTGACTATCCAAATTATGACTGAGCAGGAGTCAG
GATACTGCTGGAGAAACTTTGTGAATTATAGCCCGAGTAATGAAGCCCACTGGCCT
AGGTATCCCCATCTGTGGGTACGACTGTACGTTCTTGAACTGTACTGCATCATACT
GGGCCTGCCTCCTTGTCTCAACATTCTGAGAAGGAAGCAGCCACAGCTGACATTCT
TTACCATCGCTCTTCAGTCTTGTCATTACCAGCGACTGCCCCCACACATTCTCTGGG
CCACCGGGTTGAAAAGCGGCAGCGAGACTCCCGGGACCTCAGAGTCCGCCACACC
CGAAAGTAACACCATCAACATTGCTAAGAACGACTTCTCAGACATAGAGCTCGCG
GCTATTCCGTTCAACACCCTGGCTGACCACTACGGCGAGAGACTCGCTAGGGAGC
AGCTGGCGTTGGAGCATGAATCCTACGAGATGGGCGAGGCTAGGTTCCGCAAGAT
GTTCGAGCGACAATTGAAGGCAGGGGAGGTGGCGGACAACGCTGCCGCCAAGCCC
CTGATCACAACCTTGCTGCCCAAAATGATCGCGCGGATCAACGATTGGTTTGAGGA
GGTTAAGGCAAAACGGGGCAAACGCCCGACCGCATTTCAATTCCTCCAAGAAATC
AAGCCTGAGGCTGTTGCCTACATCACTATCAAGACGACACTGGCGTGTCTCACAAG
CGCCGACAACACCACCGTGCAAGCCGTCGCCAGCGCCATCGGGCGGGCAATTGAG
GATGAGGCACGGTTTGGTAGGATCCGAGACCTGGAAGCGAAGCACTTCAAGAAGA
ACGTGGAAGAGCAGTTGAACAAACGCGTCGGCCACGTGTATAAAAAGGCTTTCAT
GCAGGTGGTGGAGGCCGATATGCTCAGTAAGGGGCTGCTTGGGGGGGAGGCGTGG
TCATCCTGGCACAAGGAGGATAGCATTCACGTGGGGGTCCGATGTATCGAGATGC
TGATAGAGAGCACCGGAATGGTCTCCCTCCATCGCCAGAACGCTGGGGTCGTAGG
GCAGGACTCCGAGACTATTGAGCTGGCCCCCGAGTATGCCGAAGCAATCGCTACA
CGCGCAGGTGCACTGGCTGGGATAAGCCCTATGTTTCAGCCCTGCGTAGTGCCTCC
AAAGCCATGGACCGGCATCACAGGGGGTGGCTATTGGGCCAACGGTAGGCGGCCT
CTGGCCCTGGTACGCACGCACAGCAAGAAGGCGCTCATGCGCTATGAAGACGTTT
ACATGCCCGAGGTTTACAAGGCGATCAATATCGCGCAGAACACCGCCTGGAAAAT
CAATAAGAAGGTGTTGGCGGTCGCAAACGTGATTACCAAGTGGAAGCATTGCCCA
GTCGAGGACATACCCGCCATAGAACGCGAAGAGCTGCCGATGAAGCCGGAAGAC
ATTGATATGAACCCCGAGGCCCTCACCGCGTGGAAAAGAGCCGCAGCCGCCGTAT
ACAGGAAGGATAAAGCGCGCAAGTCCCGACGCATAAGCCTCGAGTTTATGCTGGA
ACAGGCCAACAAGTTCGCCAACCACAAAGCTATCTGGTTCCCCTACAACATGGACT
GGAGAGGGAGGGTCTACGCCGTCAGCATGTTCAATCCCCAGGGCAACGACATGAC
GAAGGGCCTTCTGACATTGGCAAAGGGGAAGCCTATCGGAAAGGAGGGGTACTAC
TGGCTCAAGATCCACGGCGCCAACTGCGCGGGAGTGGACAAGGTTCCATTTCCCG
AGCGAATTAAGTTCATCGAGGAAAACCACGAAAACATTATGGCGTGCGCTAAATC
CCCCCTCGAGAACACATGGTGGGCCGAGCAAGACTCCCCGTTCTGTTTTTTGGCAT
TCTGCTTTGAGTACGCCGGTGTGCAGCACCATGGCCTCTCATACAACTGTTCCCTG
CCCCTGGCCTTCGACGGAAGTTGCAGTGGGATTCAACATTTCAGCGCAATGTTGCG
GGACGAGGTCGGTGGCAGGGCCGTTAACCTGCTCCCTTCCGAAACGGTGCAGGAC
ATCTACGGAATCGTGGCAAAAAAGGTAAACGAGATCCTGCAAGCGGATGCCATCA
ACGGGACGGACAATGAGGTCGTTACGGTGACAGACGAAAATACTGGGGAAATAA
GCGAAAAGGTCAAGCTGGGGACCAAAGCACTCGCGGGTCAGTGGCTCGCCTACGG
GGTGACACGCTCCGTCACCAAGAGAAGCGTGATGACCCTCGCGTACGGTTCAAAA
GAATTCGGCTTCCGCCAGCAAGTGCTGGAGGACACCATCCAGCCGGCGATTGACT
CCGGGAAGGGTCTCATGTTTACCCAGCCGAACCAGGCCGCAGGGTACATGGCCAA
ACTGATCTGGGAAAGCGTTAGCGTCACAGTGGTCGCCGCGGTTGAGGCGATGAAT
TGGCTGAAGAGCGCGGCAAAGCTCCTCGCCGCTGAGGTGAAGGACAAAAAGACCG
GCGAAATCCTGCGCAAGCGCTGCGCCGTCCACTGGGTCACGCCGGATGGATTCCCC
GTCTGGCAGGAGTACAAGAAGCCCATCCAAACCCGGCTCAACTTGATGTTCCTTGG
CCAGTTTCGCCTGCAGCCCACGATAAACACCAACAAAGACAGCGAGATCGACGCC
CACAAGCAGGAGAGCGGCATCGCGCCCAACTTCGTGCACAGTCAGGACGGGTCCC
ATCTGCGGAAAACTGTTGTGTGGGCTCACGAGAAGTACGGCATTGAGAGCTTCGC
CCTGATACACGACAGCTTCGGGACCATACCAGCGGACGCAGCGAACCTGTTCAAA
GCCGTGCGGGAAACAATGGTCGACACCTACGAAAGCTGCGACGTACTGGCAGACT
TCTATGACCAATTCGCCGACCAGCTTCACGAGTCACAGCTCGACAAGATGCCCGCT
CTGCCCGCGAAAGGCAACCTGAATTTGCGCGACATCCTTGAGAGCGATTTTGCGTT
CGCCTCTGGTGGTTCTCCCAAGAAGAAGAGGAAAGTCTAACCGGTCATCATCACC
ATCACCATTGAGTTTAAACCCGCTGATCAGCCTCGACTGTGCCTTCTAGTTGCCAG
CCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGAC
AATAGCAGGCATGCTGGGGATGCGGTGGGCTCTATGGCTTCTGAGGCGGAAAGAA
CCAGCTGGGGCTCGATACCGTCGACCTCTAGCTAGAGCTTGGCGTAATCATGGTCA
TAGCTGTTTCCTGTGTGAAATTGTTATCCGCTCACAATTCCACACAACATACGAGC
CGGAAGCATAAAGTGTAAAGCCTAGGGTGCCTAATGAGTGAGCTAACTCACATTA
ATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCTCTTCCG
CTTCCTCGCTCACTGACTCGCTGCGCTCGGTCGTTCGGCTGCGGCGAGCGGTATCA
GCTCACTCAAAGGCGGTAATACGGTTATCCACAGAATCAGGGGATAACGCAGGAA
AGAACATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTT
CAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGG
TATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAG
TTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGTCA
TGAGATTATCAAAAAGGATCTTCACCTAGATCCTTTTAAATTAAAAATGAAGTTTT
AAATCAATCTAAAGTATATATGAGTAAACTTGGTCTGACAGTTACCAATGCTTAAT
CAGTGAGGCACCTATCTCAGCGATCTGTCTATTTCGTTCATCCATAGTTGCCTGACT
CCCCGTCGTGTAGATAACTACGATACGGGAGGGCTTACCATCTGGCCCCAGTGCTG
CAATGATACCGCGAGACCCACGCTCACCGGCTCCAGATTTATCAGCAATAAACCA
GCCAGCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAATAGTTT
GCGCAACGTTGTTGCCATTGCTACAGGCATCGTGGTGTCACGCTCGTCGTTTGGTA
TGGCTTCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTACTGTCAT
GCCATCCGTAAGATGCTTTTCTGTGACTGGTGAGTACTCAACCAAGTCATTCTGAG
AATAGTGTATGCGGCGACCGAGTTGCTCTTGCCCGGCGTCAATACGGGATAATACC
GCGCCACATAGCAGAACTTTAAAAGTGCTCATCATTGGAAAACGTTCTTCGGGGCG
AAAACTCTCAAGGATCTTACCGCTGTTGAGATCCAGTTCGATGTAACCCACTCGTG
CACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTGA
ATACTCATACTCTTCCTTTTTCAATATTATTGAAGCATTTATCAGGGTTATTGTCTC
ATGAGCGGATACATATTTGAATGTATTTAGAAAAATAAACAAATAGGGGTTCCGC
GCACATTTCCCCGAAAAGTGCCACCTGACGTCGACGGATCGGGAGATCGATCTCCC
GATCCCCTAGGGTCGACTCTCAGTACAATCTGCTCTGATGCCGCATAGTTAAGCCA
GTATCTGCTCCCTGCTTGTGTGTTGGAGGTCGCTGAGTAGTGCGCGAGCAAAATTT
AAGCTACAACAAGGCAAGGCTTGACCGACAATTGCATGAAGAATCTGCTTAGGGT
TAGGCGTTTTGCGCTGCTTCGCGATGTACGGGCCAGATATACGCGTTGACATTGAT
TATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAA
CGACCCCCGCCCATTGACGTCAATAATGACGTATGTTCCCATAGTAACGCCAATAG
GGACTTTCCATTGACGTCAATGGGTGGAGTATTTACGGTAAACTGCCCACTTGGCA
GTACATCAAGTGTATC
Rattus norvegicus APOBEC1-T7 RNA Polymerase-NLS polypeptide
sequence (SEQ ID NO: 47):
MSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIARLY
HHADPRNRQGLRDLISSGVTIQIMTEQESGYCWRNFVNYSPSNEAHWPRYPHLWVRL
YVLELYCIILGLPPCLNILRRKQPQLTFFTIALQSCHYQRLPPHILWATGLKSGSETPGTS
ESATPESNTINIAKNDFSDIELAAIPFNTLADHYGERLAREQLALEHESYEMGEARFRK
MFERQLKAGEVADNAAAKPLITTLLPKMIARINDWFEEVKAKRGKRPTAFQFLQEIKP
EAVAYITIKTTLACLTSADNTTVQAVASAIGRAIEDEARFGRIRDLEAKHFKKNVEEQL
NKRVGHVYKKAFMQVVEADMLSKGLLGGEAWSSWHKEDSIHVGVRCIEMLIESTGM
VSLHRQNAGVVGQDSETIELAPEYAEAIATRAGALAGISPMFQPCVVPPKPWTGITGG
GYWANGRRPLALVRTHSKKALMRYEDVYMPEVYKAINIAQNTAWKINKKVLAVAN
VITKWKHCPVEDIPAIEREELPMKPEDIDMNPEALTAWKRAAAAVYRKDKARKSRRIS
LEFMLEQANKFANHKAIWFPYNMDWRGRVYAVSMFNPQGNDMTKGLLTLAKGKPI
GKEGYYWLKIHGANCAGVDKVPFPERIKFIEENHENIMACAKSPLENTWWAEQDSPF
CFLAFCFEYAGVQHHGLSYNCSLPLAFDGSCSGIQHFSAMLRDEVGGRAVNLLPSETV
QDIYGIVAKKVNEILQADAINGTDNEVVTVTDENTGEISEKVKLGTKALAGQWLAYG
VTRSVTKRSVMTLAYGSKEFGFRQQVLEDTIQPAIDSGKGLMFTQPNQAAGYMAKLI
WESVSVTVVAAVEAMNWLKSAAKLLAAEVKDKKTGEILRKRCAVHWVTPDGFPVW
QEYKKPIQTRLNLMFLGQFRLQPTINTNKDSEIDAHKQESGIAPNFVHSQDGSHLRKTV
VWAHEKYGIESFALIHDSFGTIPADAANLFKAVRETMVDTYESCDVLADFYDQFADQL
HESQLDKMPALPAKGNLNLRDILESDFAFASGGSPKKKRKV
Rattus norvegicus APOBEC1-T7 RNA Polymerase-UGI-NLS plasmid
DNA sequence (SEQ ID NO: 48):
ATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTA
GTCATCGCTATTACCATGGTGATGCGGTTTTGGCAGTACATCAATGGGCGTGGATA
GCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCA
TTGACGCAAATGGGCGGTAGGCGTGTACGGTGGGAGGTCTATATAAGCAGAGCTG
GTTTAGTGAACCGTCAGATCCGCTAGAGATCCGCGGCCGCGAGAGCCGCCACCAT
GAGCTCAGAGACTGGCCCAGTGGCTGTGGACCCCACATTGAGACGGCGGATCGAG
CCCCATGAGTTTGAGGTATTCTTCGATCCGAGAGAGCTCCGCAAGGAGACCTGCCT
GCTTTACGAAATTAATTGGGGGGGCCGGCACTCCATTTGGCGACATACATCACAGA
ACACTAACAAGCACGTCGAAGTCAACTTCATCGAGAAGTTCACGACAGAAAGATA
TTTCTGTCCGAACACAAGGTGCAGCATTACCTGGTTTCTCAGCTGGAGCCCATGCG
GCGAATGTAGTAGGGCCATCACTGAATTCCTGTCAAGGTATCCCCACGTCACTCTG
TTTATTTACATCGCAAGGCTGTACCACCACGCTGACCCCCGCAATCGACAAGGCCT
GCGGGATTTGATCTCTTCAGGTGTGACTATCCAAATTATGACTGAGCAGGAGTCAG
GATACTGCTGGAGAAACTTTGTGAATTATAGCCCGAGTAATGAAGCCCACTGGCCT
AGGTATCCCCATCTGTGGGTACGACTGTACGTTCTTGAACTGTACTGCATCATACT
GGGCCTGCCTCCTTGTCTCAACATTCTGAGAAGGAAGCAGCCACAGCTGACATTCT
TTACCATCGCTCTTCAGTCTTGTCATTACCAGCGACTGCCCCCACACATTCTCTGGG
CCACCGGGTTGAAAAGCGGCAGCGAGACTCCCGGGACCTCAGAGTCCGCCACACC
CGAAAGTAACACCATCAACATTGCTAAGAACGACTTCTCAGACATAGAGCTCGCG
GCTATTCCGTTCAACACCCTGGCTGACCACTACGGCGAGAGACTCGCTAGGGAGC
AGCTGGCGTTGGAGCATGAATCCTACGAGATGGGCGAGGCTAGGTTCCGCAAGAT
GTTCGAGCGACAATTGAAGGCAGGGGAGGTGGCGGACAACGCTGCCGCCAAGCCC
CTGATCACAACCTTGCTGCCCAAAATGATCGCGCGGATCAACGATTGGTTTGAGGA
GGTTAAGGCAAAACGGGGCAAACGCCCGACCGCATTTCAATTCCTCCAAGAAATC
AAGCCTGAGGCTGTTGCCTACATCACTATCAAGACGACACTGGCGTGTCTCACAAG
CGCCGACAACACCACCGTGCAAGCCGTCGCCAGCGCCATCGGGCGGGCAATTGAG
GATGAGGCACGGTTTGGTAGGATCCGAGACCTGGAAGCGAAGCACTTCAAGAAGA
ACGTGGAAGAGCAGTTGAACAAACGCGTCGGCCACGTGTATAAAAAGGCTTTCAT
GCAGGTGGTGGAGGCCGATATGCTCAGTAAGGGGCTGCTTGGGGGGGAGGCGTGG
TCATCCTGGCACAAGGAGGATAGCATTCACGTGGGGGTCCGATGTATCGAGATGC
TGATAGAGAGCACCGGAATGGTCTCCCTCCATCGCCAGAACGCTGGGGTCGTAGG
GCAGGACTCCGAGACTATTGAGCTGGCCCCCGAGTATGCCGAAGCAATCGCTACA
CGCGCAGGTGCACTGGCTGGGATAAGCCCTATGTTTCAGCCCTGCGTAGTGCCTCC
AAAGCCATGGACCGGCATCACAGGGGGTGGCTATTGGGCCAACGGTAGGCGGCCT
CTGGCCCTGGTACGCACGCACAGCAAGAAGGCGCTCATGCGCTATGAAGACGTTT
ACATGCCCGAGGTTTACAAGGCGATCAATATCGCGCAGAACACCGCCTGGAAAAT
CAATAAGAAGGTGTTGGCGGTCGCAAACGTGATTACCAAGTGGAAGCATTGCCCA
GTCGAGGACATACCCGCCATAGAACGCGAAGAGCTGCCGATGAAGCCGGAAGAC
ATTGATATGAACCCCGAGGCCCTCACCGCGTGGAAAAGAGCCGCAGCCGCCGTAT
ACAGGAAGGATAAAGCGCGCAAGTCCCGACGCATAAGCCTCGAGTTTATGCTGGA
ACAGGCCAACAAGTTCGCCAACCACAAAGCTATCTGGTTCCCCTACAACATGGACT
GGAGAGGGAGGGTCTACGCCGTCAGCATGTTCAATCCCCAGGGCAACGACATGAC
GAAGGGCCTTCTGACATTGGCAAAGGGGAAGCCTATCGGAAAGGAGGGGTACTAC
TGGCTCAAGATCCACGGCGCCAACTGCGCGGGAGTGGACAAGGTTCCATTTCCCG
AGCGAATTAAGTTCATCGAGGAAAACCACGAAAACATTATGGCGTGCGCTAAATC
CCCCCTCGAGAACACATGGTGGGCCGAGCAAGACTCCCCGTTCTGTTTTTTGGCAT
TCTGCTTTGAGTACGCCGGTGTGCAGCACCATGGCCTCTCATACAACTGTTCCCTG
CCCCTGGCCTTCGACGGAAGTTGCAGTGGGATTCAACATTTCAGCGCAATGTTGCG
GGACGAGGTCGGTGGCAGGGCCGTTAACCTGCTCCCTTCCGAAACGGTGCAGGAC
ATCTACGGAATCGTGGCAAAAAAGGTAAACGAGATCCTGCAAGCGGATGCCATCA
ACGGGACGGACAATGAGGTCGTTACGGTGACAGACGAAAATACTGGGGAAATAA
GCGAAAAGGTCAAGCTGGGGACCAAAGCACTCGCGGGTCAGTGGCTCGCCTACGG
GGTGACACGCTCCGTCACCAAGAGAAGCGTGATGACCCTCGCGTACGGTTCAAAA
GAATTCGGCTTCCGCCAGCAAGTGCTGGAGGACACCATCCAGCCGGCGATTGACT
CCGGGAAGGGTCTCATGTTTACCCAGCCGAACCAGGCCGCAGGGTACATGGCCAA
ACTGATCTGGGAAAGCGTTAGCGTCACAGTGGTCGCCGCGGTTGAGGCGATGAAT
TGGCTGAAGAGCGCGGCAAAGCTCCTCGCCGCTGAGGTGAAGGACAAAAAGACCG
GCGAAATCCTGCGCAAGCGCTGCGCCGTCCACTGGGTCACGCCGGATGGATTCCCC
GTCTGGCAGGAGTACAAGAAGCCCATCCAAACCCGGCTCAACTTGATGTTCCTTGG
CCAGTTTCGCCTGCAGCCCACGATAAACACCAACAAAGACAGCGAGATCGACGCC
CACAAGCAGGAGAGCGGCATCGCGCCCAACTTCGTGCACAGTCAGGACGGGTCCC
ATCTGCGGAAAACTGTTGTGTGGGCTCACGAGAAGTACGGCATTGAGAGCTTCGC
CCTGATACACGACAGCTTCGGGACCATACCAGCGGACGCAGCGAACCTGTTCAAA
GCCGTGCGGGAAACAATGGTCGACACCTACGAAAGCTGCGACGTACTGGCAGACT
TCTATGACCAATTCGCCGACCAGCTTCACGAGTCACAGCTCGACAAGATGCCCGCT
CTGCCCGCGAAAGGCAACCTGAATTTGCGCGACATCCTTGAGAGCGATTTTGCGTT
CGCCTCTGGTGGTTCTACTAATCTGTCAGATATTATTGAAAAGGAGACCGGTAAGC
AACTGGTTATCCAGGAATCCATCCTCATGCTCCCAGAGGAGGTGGAAGAAGTCATT
GGGAACAAGCCGGAAAGCGATATACTCGTGCACACCGCCTACGACGAGAGCACCG
ACGAGAATGTCATGCTTCTGACTAGCGACGCCCCTGAATACAAGCCTTGGGCTCTG
GTCATACAGGATAGCAACGGTGAGAACAAGATTAAGATGCTCTCTGGTGGTTCTCC
CAAGAAGAAGAGGAAAGTCTAACCGGTCATCATCACCATCACCATTGAGTTTAAA
CCCGCTGATCAGCCTCGACTGTGCCTTCTAGTTGCCAGCCATCTGTTGTTTGCCCCT
CCCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGG
GTGGGGCAGGACAGCAAGGGGGAGGATTGGGAAGACAATAGCAGGCATGCTGGG
GATGCGGTGGGCTCTATGGCTTCTGAGGCGGAAAGAACCAGCTGGGGCTCGATAC
CGTCGACCTCTAGCTAGAGCTTGGCGTAATCATGGTCATAGCTGTTTCCTGTGTGA
AATTGTTATCCGCTCACAATTCCACACAACATACGAGCCGGAAGCATAAAGTGTA
AAGCCTAGGGTGCCTAATGAGTGAGCTAACTCACATTAATTGCGTTGCGCTCACTG
CCCGCTTTCCAGTCGGGAAACCTGTCGTGCCAGCTGCATTAATGAATCGGCCAACG
CGCGGGGAGAGGCGGTTTGCGTATTGGGCGCTCTTCCGCTTCCTCGCTCACTGACT
CGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTC
GTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCAC
TGGCAGCAGCCACTGGTAACAGGATTAGCAGAGCGAGGTATGTAGGCGGTGCTAC
AGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTGACG
CTCAGTGGAACGAAAACTCACGTTAAGGGATTTTGGTCATGAGATTATCAAAAAG
GATCTTCACCTAGATCCTTTTAAATTAAAAATGAAGTTTTAAATCAATCTAAAGTA
TATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATA
ACTACGATACGGGAGGGCTTACCATCTGGCCCCAGTGCTGCAATGATACCGCGAG
ACCCACGCTCACCGGCTCCAGATTTATCAGCAATAAACCAGCCAGCCGGAAGGGC
CGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCC
ATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATG
CTTTTCTGTGACTGGTGAGTACTCAACCAAGTCATTCTGAGAATAGTGTATGCGGC
GACCGAGTTGCTCTTGCCCGGCGTCAATACGGGATAATACCGCGCCACATAGCAG
AACTTTAAAAGTGCTCATCATTGGAAAACGTTCTTCGGGGCGAAAACTCTCAAGGA
TCTTACCGCTGTTGAGATCCAGTTCGATGTAACCCACTCGTGCACCCAACTGATCTT
CAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCC
TTTTTCAATATTATTGAAGCATTTATCAGGGTTATTGTCTCATGAGCGGATACATAT
TTGAATGTATTTAGAAAAATAAACAAATAGGGGTTCCGCGCACATTTCCCCGAAA
AGTGCCACCTGACGTCGACGGATCGGGAGATCGATCTCCCGATCCCCTAGGGTCG
ACTCTCAGTACAATCTGCTCTGATGCCGCATAGTTAAGCCAGTATCTGCTCCCTGCT
TGTGTGTTGGAGGTCGCTGAGTAGTGCGCGAGCAAAATTTAAGCTACAACAAGGC
AAGGCTTGACCGACAATTGCATGAAGAATCTGCTTAGGGTTAGGCGTTTTGCGCTG
CTTCGCGATGTACGGGCCAGATATACGCGTTGACATTGATTATTGACTAGTTATTA
ATAGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACG
TCAATGGGTGGAGTATTTACGGTAAACTGCCCACTTGGCAGTACATCAAGTGTATC
Rattus norvegicus APOBEC1-T7 RNA Polymerase-UGI-NLS polypeptide
sequence (SEQ ID NO: 49):
MSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIARLY
HHADPRNRQGLRDLISSGVTIQIMTEQESGYCWRNFVNYSPSNEAHWPRYPHLWVRL
YVLELYCIILGLPPCLNILRRKQPQLTFFTIALQSCHYQRLPPHILWATGLKSGSETPGTS
ESATPESNTINIAKNDFSDIELAAIPFNTLADHYGERLAREQLALEHESYEMGEARFRK
MFERQLKAGEVADNAAAKPLITTLLPKMIARINDWFEEVKAKRGKRPTAFQFLQEIKP
EAVAYITIKTTLACLTSADNTTVQAVASAIGRAIEDEARFGRIRDLEAKHFKKNVEEQL
NKRVGHVYKKAFMQVVEADMLSKGLLGGEAWSSWHKEDSIHVGVRCIEMLIESTGM
VSLHRQNAGVVGQDSETIELAPEYAEAIATRAGALAGISPMFQPCVVPPKPWTGITGG
GYWANGRRPLALVRTHSKKALMRYEDVYMPEVYKAINIAQNTAWKINKKVLAVAN
VITKWKHCPVEDIPAIEREELPMKPEDIDMNPEALTAWKRAAAAVYRKDKARKSRRIS
LEFMLEQANKFANHKAIWFPYNMDWRGRVYAVSMFNPQGNDMTKGLLTLAKGKPI
GKEGYYWLKIHGANCAGVDKVPFPERIKFIEENHENIMACAKSPLENTWWAEQDSPF
CFLAFCFEYAGVQHHGLSYNCSLPLAFDGSCSGIQHFSAMLRDEVGGRAVNLLPSETV
QDIYGIVAKKVNEILQADAINGTDNEVVTVTDENTGEISEKVKLGTKALAGQWLAYG
VTRSVTKRSVMTLAYGSKEFGFRQQVLEDTIQPAIDSGKGLMFTQPNQAAGYMAKLI
WESVSVTVVAAVEAMNWLKSAAKLLAAEVKDKKTGEILRKRCAVHWVTPDGFPVW
QEYKKPIQTRLNLMFLGQFRLQPTINTNKDSEIDAHKQESGIAPNFVHSQDGSHLRKTV
VWAHEKYGIESFALIHDSFGTIPADAANLFKAVRETMVDTYESCDVLADFYDQFADQL
HESQLDKMPALPAKGNLNLRDILESDFAFASGGSTNLSDIIEKETGKQLVIQESILMLPE
EVEEVIGNKPESDILVHTAYDESTDENVMLLTSDAPEYKPWALVIQDSNGENKIKMLS
GGSPKKKRKV

### Uracil Glycosylase Inhibitor

In certain aspects, the compositions of the instant disclosure include a uracil glycosylate inhibitor. Uracil glycosylate inhibitor has been shown to facilitate C:G→T:A mutations. Uracil glycosylate inhibitor or uracil-DNA glycosylase inhibitor (UGI) is a small protein from Bacillus subtilis bacteriophage PBS1 which inhibits E. coli and other species' uracil DNA glycosylase (UDG). UGI can disassociate UDG: DNA complexes. This protein binds specifically and reversibly to the host uracil-DNA glycosylase, preventing removal of uracil residues from PBS2 DNA by the host uracil-excision repair system. An exemplary UGI sequence is:

Bacillus subtilis Uracil glycosylate inhibitor
(SEQ ID NO: 21)
MTNLSDIIEKETGKQLVIQESILMLPEEVEEVIGNKPESDILVHTAYDE
STDENVMLLTSDAPEYKPWALVIQDSNGENKIKML

### Nuclear Localization Signals (NLS)

In some aspects, the compositions of the present disclosure include a pEditor containing the T7 RNAP-cytidine deaminase fusion gene with a nuclear localization signal. A nuclear localization signal or sequence (NLS) is an amino acid sequence that ‘tags’ a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. (Kalderon et al. Cell. 39: 499-509).

Classical NLSs can be classified as either monopartite or bipartite. The major structural differences between the two is that the two basic amino acid clusters in bipartite NLSs are separated by a relatively short spacer sequence (hence bipartite—2 parts), while monopartite NLSs are not. The first NLS to be discovered was the sequence PKKKRKV (SEQ ID NO: 22) in the SV40 Large T-antigen (a monopartite NLS; Kalderon et al. Cell. 39: 499-509). The NLS of nucleoplasmin, KR[PAATKKAGQA]KKKK (SEQ ID NO: 23), is the prototype of the ubiquitous bipartite signal: two clusters of basic amino acids, separated by a spacer of about 10 amino acids (Dingwall et al. J. Cell Biol. 107: 841-9). Both signals are recognized by importin α. Importin α contains a bipartite NLS itself, which is specifically recognized by importin β. The latter can be considered the actual import mediator.

Chelsky et al. proposed the consensus sequence K-K/R-X-K/R (SEQ ID NO: 24) for monopartite NLSs (Dingwall et al.). A Chelsky sequence may, therefore, be part of the downstream basic cluster of a bipartite NLS. Makkerh et al. carried out comparative mutagenesis on the nuclear localization signals of SV40 T-Antigen (monopartite), C-myc (monopartite), and nucleoplasmin (bipartite), and showed amino acid features common to all three. The role of neutral and acidic amino acids was shown for the first time in contributing to the efficiency of the NLS (Makkerh et al. Curr. Biol. 6: 1025-7).

Rotello et al. compared the nuclear localization efficiencies of eGFP fused NLSs of SV40 Large T-Antigen, nucleoplasmin (AVKRPAATKKAGQAKKKKLD; SEQ ID NO: 25), EGL-13 (MSRRRKANPTKLSENAKKLAKEVEN; SEQ ID NO: 26), c-Myc (PAAKRVKLD; SEQ ID NO: 27) and TUS-protein (KLKIKRPVK; SEQ ID NO: 28) through rapid intracellular protein delivery. They found significantly higher nuclear localization efficiency of c-Myc NLS compared to that of SV40 NLS (Ray et al. Bioconjug. Chem. 26: 1004-7).

### Mammalian Expression Vector Promoters

An expression vector, otherwise known as an expression construct, is commonly a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and promoter regions and lead to efficient transcription of the gene carried on the expression vector. The promoters for cytomegalovirus (CMV) and SV40 are commonly used in mammalian expression vectors to drive gene expression. Non-viral promoter, such as the elongation factor (EF)-1 promoter, is also known.

CMV Promoter is commonly included in vectors used in genetic engineering work conducted in mammalian cells, as it is a strong promoter that drives constitutive expression of genes under its control. This promoter has been used to express a plethora of eukaryotic gene products and is used for specialty protein production, gene therapy, and DNA-based vaccination, among other applications.

The CMV promoter has the following sequence (SEQ ID NO: 29):

TAGTAATCAATTACGGGGTCATTAGTTCATAGCCCATATATGGAGTTCC
GCGTTACATAACTTACGGTAAATGGCCCGCCTGGCTGACCGCCCAACGA
CCCCCGCCCATTGACGTCAATAATGACGTATGTTCCCATAGTAACGCCA
ATAGGGACTTTCCATTGACGTCAATGGGTGGAGTATTTACGGTAAACTG
CCCACTTGGCAGTACATCAAGTGTATCATATGCCAAGTACGCCCCCTAT
TGACGTCAATGACGGTAAATGGCCCGCCTGGCATTATGCCCAGTACATG
ACCTTATGGGACTTTCCTACTTGGCAGTACATCTACGTATTAGTCATCG
CTATTACCATGGTGATGCGGTTTTGGCAGTACATCAATGGGCGTGGATA
GCGGTTTGACTCACGGGGATTTCCAAGTCTCCACCCCATTGACGTCAAT
GGGAGTTTGTTTTGGCACCAAAATCAACGGGACTTTCCAAAATGTCGTA
ACAACTCCGCCCCATTGACGCAAATGGGCGGTAGGCGTGTACGGTGGGA
GGTCTATATAAGCAGAGCTGGTTTAGTGAACCGTCAG

SV40 Promoter (Simian Virus 40 promoter) contains the SV40 enhancer promoter region and origin of replication (part no. GA-ori-00009.1) for high-level expression and replication in cell lines expressing the large T antigen (e.g. COS-7 and 293T cells). It does not replicate episomally in the absence of the SV40 large T antigen. The SV40 promoter is weak in B cells, but SV40 exhibits high activity in T24 and HCV29 human bladder urethelium carcinoma cell lines.

Human elongation factor-1 alpha (EF-1 alpha) or EF-1 is a constitutive non-viral promoter of human origin that can be used to drive ectopic gene expression in various in vitro and in vivo contexts. EF-1 alpha is often useful in conditions where other promoters (such as CMV) have diminished activity or have been silenced (as in embryonic stem cells).

### Directed Evolution

Directed evolution (DE) is a method used in protein engineering that mimics the process of natural selection to steer proteins or nucleic acids toward a user-defined goal. In general, DE involves subjecting a gene to iterative rounds of mutagenesis, selection (expressing those variants and isolating members with the desired function), and amplification (generating a template for the next round). Advantageously, it can be performed both in vivo and in vitro). Directed evolution is used both for protein engineering as an alternative to rationally designing modified proteins, as well as studies of fundamental evolutionary principles in a controlled, laboratory environment.

Mammalian cells have been employed in DE to engineer recombinant proteins, particularly those that require posttranslational modifications, such as antibodies, hormones and cytokines. Bacteria and yeast are less suitable to evolve these types of proteins because they have insufficient disulfide-bridge formation mechanisms, lack glycosylation, and frequently form protein aggregates. The ability to evolve mammalian proteins within mammalian cells is a relatively recent development, with the methods of the instant disclosure constituting an advance in mammalian mutagenesis approaches available for performing DE. Enhanced performance of DE in mammalian cells is expected to decrease the development time required for generating robust, high-producing mammalian cells lines for commercial applications involving engineering of novel enzymes, proteins (e.g., pharmaceutical applications), and immune support therapies (e.g., bacteriophage with antibody genes). As compared to bacteria and yeast, mammalian cells exhibit low productivity due to their slow growth rates and tendency to undergo programmed cell death (apoptosis). DE in mammalian cells has previously relied upon non-physiological environments, with such DE methods rapidly saturating mutagenized sites, or such DE approaches have only been adapted optimally in bacterial and yeast systems. Use of DE in mammalian cells prior to the instant disclosure has also been hampered because mammalian cells are time-consuming to work with, exhibit a low efficiency of stable gene integration, have a tendency toward multiple gene insertions, and display highly variable expression levels. Certain aspects of the instant disclosure relate to compositions and methods that involve pseudo-random integrated mutation of eukaryotic cells (PRIME), which enables DE in mammalian cells while overcoming some of the above-stated challenges to DE previously described in the art (Pourmir et al. Comput Struct Biotechnol J. 2: e201209012).

### Mammalian Target Genes

The methods and compositions of the instant disclosure can be applied to achieve targeted mutagenesis of mammalian cells across long stretches of sequence, optionally in and around effectively any region of the genome, including targeted genes and/or other genetic elements. In certain embodiments, the methods and compositions of the instant disclosure can be applied to oncogenes and/or cancer-related genes. Exemplary oncogenes and/or cancer-related genes include, but are not limited to, those recited in Table 1.

TABLE 1
Exemplary Oncogenes and Cancer-Related Genes

| | | | | |
|---|---|---|---|---|
| ABL1 | FLT3 | MCL1 | PRKCQ | WEE1 |
| ABL2 | FNTA | MDM2 | PRKCSH | XI4P |
| AKT1 | GSK3A | MEK1 | PRKCZ | |
| AKT2 | GSK3B | MET | PRKDC | |
| AKT3 | HDAC1 | MTOR | PSENEN | |
| ALK | HDAC2 | NFKB1 | PSMB5 | |
| AR | HDAC3 | NTRK1 | PTK2 | |
| ATM | HDAC6 | P4HB | PTPN11 | |
| AURKA | HDAC8 | p53 | PTPN6 | |
| AURKB | HER2 | PAK1 | RAC1 | |
| AURKC | HSP90AA1 | PARP1 | RET | |
| BCL2 | HSP90AB1 | PDGFRA | ROCK1 | |
| BCL-ABL1 | HSP90AB4P | PDGFRB | ROCK2 | |
| BMX | HSP90B1 | PDK1 | RPS6KA1 | |
| BRAF | HSP90B3P | PIK3CA | RPS6KA2 | |
| BTK | IGF1R | PIK3CB | RPS6KA3 | |
| CASP3 | IKBKE | PIK3CD | RPS6KA4 | |
| CCR5 | ITK | PIK3CG | RPS6KA5 | |
| CDK1 | JAK2 | PLK1 | RPS6KA6 | |
| CDK2 | KDR | PLK2 | RPS6KB2 | |
| CDK4 | KIT | PLK3 | RXRA | |
| CDK6 | KRAS | PPM1D | RXRB | |
| CDK7 | MAP2K1 | PRKAA1 | SGK3 | |
| CTNNB1 | MAP2K2 | PRKCA | SMO | |
| DHFR | MAPK11 | PRKCB | SRC | |
| EGFR | MAPK12 | PRKCD | SYK | |
| ERBB2 | MAPK13 | PRKCE | TBK1 | |
| FGFR1 | MAPK14 | PRKCG | TEC | |
| FGFR3 | MAPK7 | PRKCH | TNF | |
| FLT1 | MAPK8 | PRKCI | TOP1 | |

### Mammalian Cell Culture

In certain aspects, the instant disclosure describes methods and compositions designed to achieve targeted mutagenesis of mammalian cells across long stretches of sequence. Mammalian cell culture is used widely in academic, medical and industrial settings. It has provided a means to study the physiology and biochemistry of the cell, and developments in the fields of cell and molecular biology have required the use of reproducible model systems, which cultured cell lines are especially capable of providing. For medical use, cell culture provides test systems to assess the efficacy and toxicology of potential new drugs. Large-scale mammalian cell culture has allowed production of biologically active proteins, initially production of vaccines and then recombinant proteins and monoclonal antibodies; meanwhile, recent innovative uses of cell culture include tissue engineering, as a means of generating tissue substitutes.

Mammalian cells can be isolated from tissues for ex vivo culture in several ways. Cells can be easily purified from blood. However, only the white cells are capable of growth in culture. Cells can be isolated from solid tissues by digesting the extracellular matrix using enzymes such as collagenase, trypsin, or pronase, before agitating the tissue to release the cells into suspension. Alternatively, pieces of tissue can be placed in growth media, and the cells that grow out are available for culture. This method is known as explant culture. Cells that are cultured directly from a subject are known as primary cells. With the exception of some derived from tumors, most primary cell cultures have limited lifespan (Voight et al. Journal of Molecular and Cellular Cardiology. 86: 187-98). An established or immortalized cell line has acquired the ability to proliferate indefinitely either through random mutation or deliberate modification, such as artificial expression of the telomerase gene. Numerous cell lines are well established as representative of particular cell types. Examples of commonly used mammalian cell lines include HEK293T cells, VERO, BHK, HeLa, CV1 (including Cos), MDCK, 293, 3T3, myeloma cell lines (e.g., NSO, NS 1), PC12, W138 cells, and Chinese hamster ovary (CHO) cells, among many other examples (Langdon et al. Molecular Biomethods Handbook. 861-873).

### Mammalian Cell Transfection Methods

Mammalian cell transfection is a technique commonly used to express exogenous DNA or RNA in a host cell line. There are many different methods available for transfecting mammalian cells, depending upon the cell line characteristics, desired effect, and downstream applications. These methods can be broadly divided into two categories: those used to generate transient transfection, and those used to generate stable transfectants. Transient transfection methods include, but are not limited to, liposome-mediated transfection, non-liposomal transfection agents (lipids and polymers), dendrimer-based transfection, and electroporation. Stable transfection methods include, but are not limited to microinjection, and virus-mediated gene delivery.

Certain aspects of the instant disclosure describe methods and compositions designed to achieve targeted mutagenesis in mammalian cells across long stretches of sequence, via use of virus-mediated gene delivery (bacteriophages). Viral vectors, such as bacteriophages, retrovirus, adenovirus (types 2 and 5), adeno-associated virus, herpes virus, pox virus, human foamy virus (HFV), and lentivirus have been used for gene transfection. All viral vector genomes have been modified by deleting some areas of their genomes so that their replication becomes altered, rendering such viruses safer than native forms. However, viral delivery systems have some problems, including: the marked immunogenicity of viruses, which can cause induction of the inflammatory system, potentially leading to degeneration of transducted tissue; and toxin production, including mortality, the insertional mutagenesis; and their limitation in transgenic capacity size. During the past few years some viral vectors with specific receptors have been designed that are capable of transferring transgenes to some other specific cells, which are not their natural target cells (retargeting) (Nayerossadat et al. Adv Biomed Res. 1: 27).

### Kits

The instant disclosure also provides kits containing compositions of the instant disclosure, e.g., for use in methods of the present disclosure. Kits of the instant disclosure may include one or more containers comprising a composition (e.g., a nucleic acid encoding for a nucleic acid-editing deaminase and a bacteriophage RNA polymerase (e.g., T7 RNAP), optionally also encoding for a UGI and/or a NLS) of this disclosure. In some embodiments, the kits further include instructions for use in accordance with the methods of this disclosure. In some embodiments, these instructions comprise a description of administration/transfection of the composition(s) to mammalian cells, optionally further including instructions for performance of directed evolution of a targeted gene in mammalian cell(s).

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a mammalian cell transfection agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

Design and Construction of pTarget and pEditor Plasmids

A list of the plasmids and primers used in this disclosure are listed in Table 2.

TABLE 2
Plasmids and Primers of the Disclosure
Plasmids

| Name | Description |
|---|---|
| pTarget | T7 promoter- EGFP |
| pTarget-CMV | CMV promoter- T7 promoter-EGFP |
| pTarget-CMV-EBFP | CMV promoter- T7 promoter-BFP |
| pTarget-no T7pro | Deleting T7 promoter in pTarget |
| pT7 | T7RNAP only |
| pAID | AID*Δ only |
| pAPOBEC-T7 | Rat APOBEC1-T7 RNAP |
| pAPOBEC-T7-UGI | Rat APOBEC1-T7 RNAP-UGI |
| pAID-T7 | AID*Δ-T7 RNAP |
| pAID-T7-UGI | AID*Δ-T7 RNAP-UGI |
| pAID-T7G645A-UGI | AID*Δ-T7 RNAP G645A-UGI |
| pAID-T7P266L-UGI | AID*Δ-T7 RNAP P266L-UGI |
| pAID-T7P266LG645A-UGI | AID*Δ-T7 RNAP P266L G645A-UGI |
| pAID-T7G645AQ744R-UGI | AID*Δ-T7 RNAP G645A Q744R-UGI |
| Lenti_CMV_T7_GFP-T-IR | CMV promoter- T7 promoter-EGFP |
| | in Lentiviral backbone |

Cloning Primers

| Vector | Direction | Sequence (5′-3′) | Description |
|---|---|---|---|
| pCMV | Forward | TGAGAGCGATTTTGCGTTCGCCTCTGGTGGTTCTCCC | To ampify the |
| | | AAGAAG (SEQ ID NO: 50) | backbone for |
| | | | pAPOBEC-T7 |
| pCMV | Reverse | GTTCTTAGCAATGTTGATGGTGTTACTTTCGGGTGTGG | To ampify the |
| | | CGGACTC (SEQ ID NO: 51) | backbone for |
| | | | pAPOBEC-T7 |
| pCMV | Forward | GAGTCCGCCACACCCGAAAGTAACACCATCAACATTG | To ampify the |
| | | CTAAGAAC (SEQ ID NO: 52) | insert for |
| | | | pAPOBEC-T7 |
| pCMV | Reverse | CTTCTTGGGAGAACCACCAGAGGCGAACGCAAAATCG | To ampify the |
| | | CTCT (SEQ ID NO: 53) | insert for |
| | | | pAPOBEC-T7 |
| pCMV | Forward | TCTGGTGGTTCTCCCAAGAAGAAG (SEQ ID NO: 54) | To ampify the |
| | | | backbone for pAID |
| pCMV | Reverse | GGTGGCGGCTCTCGCGGC (SEQ ID NO: 55) | To ampify the |
| | | | backbone for pAID |
| pCMV | Forward | cggccgcgagagccgccaccATGGACAGCCTCTTGATG (SEQ | To ampify the |
| | | ID NO: 56) | insert for pAID |
| pCMV | Reverse | ttcttgggagaaccaccagaAGTACGAAATGCGTCTCG (SEQ ID | To ampify the |
| | | NO: 57) | insert for pAID |
| pCMV | Forward | AGAGCGATTTTGCGTTCGCCTCTGGTGGTTCTACTAAT | To ampify the |
| | | CTGTCAG (SEQ ID NO: 58) | backbone for |
| | | | pAPOBEC-17-UGI |
| pCMV | Reverse | GTTCTTAGCAATGTTGATGGTGTTACTTTCGGGTGTGG | To ampify the |
| | | CGGA (SEQ ID NO: 59) | backbone for |
| | | | pAPOBEC-17-UGI |
| pCMV | Forward | GAGTCCGCCACACCCGAAAGTAACACCATCAACATTG | To ampify the |
| | | CTAAGAAC (SEQ ID NO: 60) | insert for |
| | | | pAPOBEC-17-UGI |
| pCMV | Reverse | CAGATTAGTAGAACCACCAGAGGCGAACGCAAAATCG | To ampify the |
| | | CTCT (SEQ ID NO: 61) | insert for |
| | | | pAPOBEC-17-UGI |
| pCMV | Forward | TACGAGACGCATTTCGTACTAGCGGCAGCGAGACTCC | To ampify the |
| | | CG (SEQ ID NO: 62) | backbone for |
| | | | pAID-17/pAID-17-UGI |
| pCMV | Reverse | GGTTCATCAAGAGGCTGTCCATGGTGGCGGCTCTCCC | To ampify the |
| | | TATAG (SEQ ID NO: 63) | backbone for |
| | | | pAID-17/pAID-17-UGI |
| pCMV | Forward | TATAGGGAGAGCCGCCACCATGGACAGCCTCTTGATG | To ampify the |
| | | AACC (SEQ ID NO: 64) | insert for pAID-17/ |
| | | | pAID-17-UGI |
| pCMV | Reverse | CCGGGAGTCTCGCTGCCGCTAGTACGAAATGCGTCTC | To ampify the |
| | | GTAAGT (SEQ ID NO: 65) | insert for pAID-17/ |
| | | | pAID-17-UGI |
| pCMV | Forward | TCTGGTGGTTCTACTAATCTG (SEQ ID NO: 66) | To ampify the |
| | | | backbone for |
| | | | pAID-T7G645A-UGI |
| pCMV | Reverse | ACTTTCGGGTGTGGCGGA (SEQ ID NO: 67) | To ampify the |
| | | | backbone for |
| | | | pAID-T7G645A-UGI |
| pCMV | Forward | agtccgccacacccgaaagtAACACCATCAACATTGCTAAGAA | To ampify the |
| | | C (SEQ ID NO: 68) | insert for pAID- |
| | | | 17G645A-UGI |
| pCMV | Reverse | agattagtagaaccaccagaGGCGAACGCAAAATCGCTC (SEQ | To ampify the |
| | | ID NO: 69) | insert for pAID- |
| | | | 17G645A-UGI |
| pCMV | Forward | TTATGTTTCAGCCCTGCG (SEQ ID NO: 70) | To ampify the |
| | | | backbone for |
| | | | pAID-17P266L-UGI/ |
| | | | pAID-17P266LG645A-UGI |
| pCMV | Reverse | ACTTTCGGGTGTGGCGGA (SEQ ID NO: 71) | To ampify the |
| | | | backbone for |
| | | | pAID-17P266L-UGI/ |
| | | | pAID-17P266LG645A-UGI |
| PCMV | Forward | agtccgccacacccgaaagtAACACCATCAACATTGCTAAGAA | To ampify the |
| | | C (SEQ ID NO: 72) | insert for pAID- |
| | | | T7P266L-UGI/ |
| | | | pAID-T7P266LG645A-UGI |
| pCMV | Reverse | tacgcagggctgaaacataaGGCTTATCCCAGCCAGTG (SEQ | To ampify the |
| | | ID NO: 73) | insert for pAID- |
| | | | 17P266L-UGI/ |
| | | | pAID-17P266LG645A-UGI |
| pCMV | Forward | CCTTGAGAGCGATTTTGC (SEQ ID NO: 74) | To ampify the |
| | | | backbone for |
| | | | pAID-17G645AQ744R-UGI |
| pCMV | Reverse | GGATGGGCTTCTTGTACTC (SEQ ID NO: 75) | To ampify the |
| | | | backbone for |
| | | | pAID-17G645AQ744R-UGI |
| pCMV | Forward | ggagtacaagaagcccatccGAACCCGGCTCAACTTGATG | To ampify the |
| | | (SEQ ID NO: 76) | insert for pAID- |
| | | | 17G645AQ744R-UGI |
| pCMV | Reverse | acgcaaaatcgctctcaaggATGTCGCGCAAATTCAG (SEQ | To ampify the |
| | | ID NO: 77) | insert for pAID- |
| | | | 17G645AQ744R-UGI |
| pUC19 | Forward | attcgagctcggtacccgggTAATACGACTCACTATAGGC (SEQ | To ampify the |
| | | ID NO: 78) | insert for pTarget |
| | | | (restriction |
| | | | enzyme cloning, |
| | | | no need to amplify |
| | | | the backbone) |
| pUC20 | Reverse | gccaagcttgcatgcctgcaAGGGAAGAAAGCGAAAGG (SEQ | To ampify the |
| | | ID NO: 79) | insert for pTarget |
| | | | (restriction |
| | | | enzyme cloning, |
| | | | no need to amplify |
| | | | the backbone) |
| pcDNA | Forward | CCATCGATGAGACCCAAGCTGGCTAGC (SEQ ID NO: | To delete the 17 |
| 3.1 (+) | | 80) | promoter in |
| | | | pTarget-CMV |
| pcDNA | Reverse | CCATCGATATTTCGATAAGCCAGTAAGCAGTGG (SEQ | To delete the 17 |
| 3.1 (+) | | ID NO: 81) | promoter in |
| | | | pTarget-CMV |
| pcDNA | Forward | TGAATTAATTAAGAATTATCACCGCTTC (SEQ ID NO: 82) | To ampify the |
| 3.1 (+) | | | backbone for |
| | | | pTarget-CMV-BFP |
| pcDNA | Reverse | CTAGTGGATCCGAGCTCG (SEQ ID NO: 83) | To ampify the |
| 3.1 (+) | | | backbone for |
| | | | pTarget-CMV-BFP |
| pcDNA | Forward | accgagctcggatccactagATGGTGAGCAAGGGCGAG (SEQ | To ampify the |
| 3.1 (+) | | ID NO: 84) | insert for pTarget- |
| | | | CMV-BFP |
| pcDNA | Reverse | tgataattcttaattaattcaTTACTTGTACAGCTCGTCCATG | To ampify the |
| 3.1 (+) | | (SEQ ID NO: 85) | insert for pTarget- |
| | | | CMV-BFP |
| Lenti_ | Forward | AATTCGAAGCTTGAGCTCG (SEQ ID NO: 86) | To ampify the |
| CMV_T_ | | | backbone for |
| IR | | | Lenti_CMV_T7_ |
| | | | GFP-T-IR |
| Lenti_ | Reverse | ACTAGTTCTAGAGTCGGTG (SEQ ID NO: 87) | To ampify the |
| CMV_T_ | | | backbone for |
| IR | | | Lenti_CMV_T7_ |
| | | | GFP-T-IR |
| Lenti | Forward | acaccgactctagaactagtTAATACGACTCACTATAGGG (SEQ | To ampify the |
| CMV_T_ | | ID NO: 88) | insert for |
| IR | | | Lenti_CMV_T7_ |
| | | | GFP-T-IR |
| Lenti_ | Reverse | tcgagctcaagcttcgaattTTTATTAGGAAAACAACAGATG | To ampify the |
| CMV_T_ | | (SEQ ID NO: 89) | insert for |
| IR | | | Lenti_CMV_T7_ |
| | | | GFP-T-IR |

Amplification Primers

| Target name | Direction | Sequence (5′-3′) |
|---|---|---|
| GFP/BFP | Forward | ATGGTGAGCAAGGGCGAGGA (SEQ ID |
| | | NO: 90) |
| GFP/BFP | Reverse | TTACTTGTACAGCTCGTCCATGC (SEQ |
| | | ID NO: 91) |
| 2000-bp region in pTarget | Forward | GCAAATGGGCGGTAGGCGT (SEQ ID |
| (pcDNA3.1-IRES-EGFP) | | NO: 92) |
| 2000-bp region in pTarget | Reverse | GGCGCTGGCAAGTGTAGCG (SEQ ID |
| (pcDNA3.1-IRES-EGFP) | | NO: 93) |
| 2000-bp region in pTarget | Forward | AACTAGAGAACCCACTGCTTACTG |
| (pcDNA3.1-noCMV-IRES- | | (SEQ ID NO: 94) |
| EGFP) | | |
| 2000-bp region in pTarget | Reverse | GGCGCTGGCAAGTGTAGCG (SEQ ID |
| (pcDNA3.1-noCMV-IRES- | | NO: 95) |
| EGFP) | | |
| Chr6 | Forward | TCAGACAACCTCATTTCC (SEQ ID NO: |
| | | 96) |
| Chr6 | Reverse | GCTTACTACAACTTTTAAAAGTT (SEQ |
| | | ID NO: 97) |
| Chr7 | Forward | TCACCAGTCGTTTTTCAGAT (SEQ ID |
| | | NO: 98) |
| Chr7 | Reverse | CCATACTCCTTTTAAAAATATAATACAAC |
| | | (SEQ ID NO: 99) |
| Upstream-T7pro- | Forward_1 | GATCTTCAGACCTGGAGGA (SEQ ID |
| downstream (designed | | NO: 100) |
| based on Lenti-T7pro- | | |
| EGFP) | | |
| Upstream-T7pro- | Reverse | TAGAAGGCACAGTCGAGG (SEQ ID NO: |
| downstream (designed | | 101) |
| based on Lenti-T7pro- | | |
| EGFP) | | |
| Upstream-T7pro- | Forward_2 | GAACAGGGACTTGAAAGCGA (SEQ ID |
| downstream (designed | | NO: 102) |
| based on Lenti-T7pro- | | |
| EGFP) | | |
| Upstream-T7pro- | Reverse | TAGAAGGCACAGTCGAGG (SEQ ID NO: |
| downstream (designed | | 103) |
| based on Lenti-T7pro- | | |
| EGFP) | | |

pcDNA3.1(+)-IRES-GFP was a gift from Kathleen L. Collins (Addgene plasmids #51406). pCMV-BE3 was a gift from David Liu (Addgene plasmid #73021). pGH335_MS2-AID*Δ-Hygro was a gift from Michael Bassik (Addgene plasmid #85406). Lenti_CMV_T_IR, Lenti_PAX2 and Lenti_VSVg were gifts from Jamie Marshall. T7 RNAP was ordered as a gBlock from Integrated DNA Technologies (IDT). The Cas9(D10A) in the pCMV-BE3 construct was replaced with T7 RNAP by Gibson assembly to generate pAPOBEC-T7 and pAPOBEC-T7-UGI in which the original T7 promoter was also deleted to avoid self-editing. Rat APOBEC1 in pAPOBEC-T7 and pAPOBEC-T7-UGI was replaced with AID*A amplified from pGH335_MS2-AID*Δ-Hygro to generate pAID-T7 and pAID-T7-UGI. For pTarget, T7 promoter-GFP fragment was amplified from pcDNA3.1(+)-IRES-GFP and was sub-cloned into a pUC19 backbone. This fragment was also sub-cloned into the Lenti_CMV-T-IR to generate the Lenti_CMV_T7_GFP-T-IR. A pTarget plasmid without T7 promoter was also cloned as a negative control. BFP fragment was generated from GFP sequence via site-directed mutagenesis. pAID-T7G645A-UGI, pAID-T7P266L-UGI, pAID-T7P266LG645A-UGI and pAID-T7G645AQ744R-UGI were cloned via site-directed mutagenesis using wild type pAID-T7-UGI as a template. All plasmid sequences were verified using Sanger sequencing. All cloning primers were ordered from IDT. Plasmids were extracted using Qiaprep® Spin Miniprep Kit and Plasmid Plus Midi Kit (Qiagen®).

### Cell Culture and Plasmid Transfection

HEK293T cells were obtained from ATCC and were grown in high-glucose (4.5 g/L) DMEM supplemented with GlutaMAX™, 1 mM sodium pyruvate, 10% FBS, 100 units/mL of penicillin and 100 μg/mL of streptomycin in a humidified chamber with 5% CO₂ at 37° C. Cells were maintained at ˜80% confluence in 24-well plates on the day of transfection. 250 ng of pTarget and 250 ng of pEditor plasmids were mixed together with 1 μl of TransIT-X2 reagent (Mirus) and the mixture was incubated in 50 μl of Opti-MEM® (Thermo Fisher Scientific™) for 30 min. The mixture was then added drop-wise to each well. For time-point experiment using target-integrated single cell clones, cells were cultured in 12-well plates and were transfected with 1000 ng of pTarget plasmids. Cells were subsequently harvested at the time points indicated above.

### Lentivirus Production and Generation of Single Cell Clones

3 million HEK293T cells were cultured in 10 mL of culture media in a 10-cm dish. Cells were transfected with 12 μg of Lenti_CMV_T7_GFP-T-IR, 9 μg of Lenti_PAX2 and 3 μg of lenti_VSVg. 24 hr after transfection, culture media was replaced with 6 mL of high-glucose (4.5 g/L) DMEM supplemented with GlutaMAX™, 1 mM sodium pyruvate, 30% FBS, 100 units/mL of penicillin and 100 μg/mL of streptomycin. Supernatant containing viral particles was collocated and filtered through 0.22 μM filters 24 hr after. To generate single cell clones, HEK293T cells in a 6-well plate with 2.5 mL of culture media received 500 μl of virus together with polybrene at a final concentration of 8 μg/mL. Two days after transduction, successfully-integrated cells were selected by puromycin at a concentration of 1.5 μg/mL. Seven days after transduction, integrated cells were subject to FACS-sorting in single cell format into 96-well plates using a MoFlo® Astrios™ EQ Cell Sorter (Beckman Coulter™) and single cells were allowed to expand to form colonies.

### Fluorescence Microscopy and Image Analysis

HEK293T cells transfected with pTarget and pEditor plasmids were seeded in a 24-well glassbottom plate. Cells were imaged using an inverted Nikon® CSU-W1 Yokogawa® spinning disk confocal microscope with 488 nm (GFP) and 405 nm (BFP) lasers, an air objective (Plan Apo λ, numerical aperture (NA)=0.75, 20×, Nikon), and an Andor® Zyla sCMOS® camera. NIS-Elements AR software (v4.30.01, Nikon®) was used for image capture. Images were processed using ImageJ (National Institutes of Health). CellProfiler (version 3.1.5, Broad Institute) (21) was used for segmentation and counting BFP and GFP positive cells. GFP positive cells were further thresholded by Otsu's method using integrated intensity with the R package autothresholdr (22).

### Preparation of Sequencing Library

To sequence the targeted region (˜2000 bp) on pTarget, plasmids were extracted from ˜1 million cells using Qiaprep Spin Miniprep Kit. PCR was performed using those plasmids as templates (primer sequences are shown in Table 2 above. Ampure® XP beads (Beckman Coulter™) were added to samples at a 0.8:1 ratio to size select for the pcr'ed fragments. The concentration of each sample was measured by Qubit™ (Thermo Fisher Scientific™). 1 ng of DNA at a volume of 2.5 μl from each sample was used as input for the subsequent library preparation. Sequencing library was prepared following the Nextera® XT Kit protocol (Illumina®) except that half the amount of each reagent was used. To sequence the targeted loci, genomic DNA was extracted from ˜1 million cells using the Quick-DNA™ Kit (Zymo Research™). 4 μl of extracted genomic DNA were used to set up in vitro transcription reactions at a volume of 10 μl using HiScribe™ T7 High Yield RNA Synthesis Kit (New England BioLabs, Inc.®). The newly synthesized RNA was purified using RNA Clean & Concentrator Kit (Zymo Research™). Reverse transcription was performed using SuperScript® IV First-Strand Synthesis System (Thermo Fisher Scientific™) cDNA was purified using AMPure® XP beads at a ratio of 1:1 and was used as the template for subsequent PCR reactions. The concentration of each sample was measured by Qubit® and the same Nextera® XT Kit protocol was followed to prepare sequencing library. Sequences were measured on a MiSeq® (Illumina®) with paired-end reads.

### Analysis of Sequencing Data

On average, 1 million reads were produced for each sample. Illumina® sequencing adapters were trimmed during sample demultiplexing using bcl2fastq2 (version 2.19.1). Bases in each read with Illumina® quality score lower than 25 were filtered. Alignment on respective reference sequence was performed using Bowtie 2 (v2.2.4.1) (23). Alignment files were generated in bam format and were visualized in Geneious (v11.1.5). The mutation enrichment was calculated at each base with custom Matlab™ scripts. The first and last 15 bases of each aligned read and bases with read count less than 100 were excluded from the analysis. Transitions, transversions, and indels observed at each position were calculated, and the C->T and G->A mutation profiles were plotted, respectively, for each sample. The mutation rate per base data was obtained by dividing the number of reads with mutations over the number of total reads at each base. The average mutation rate for each possible combination of base switching for each sample was calculated by averaging the mutation rate per base data across the targeted region. The pT7 sample was used to estimate the background error rates introduced through sample preparation and Illumina® sequencing. The final average mutation rate for each base switching combination was calculated by subtracting the background error rate. Negative values were set to 0. All bar graphs and dot plots were generated in RStudio® using ggplot2.

### Statistical Analysis

Pairwise comparison was analyzed using two-sided t test.

### Example 2: Construction and Demonstration of a Pseudo-Random Integrated Mutation of Eukaryotic Cells (PRIME)

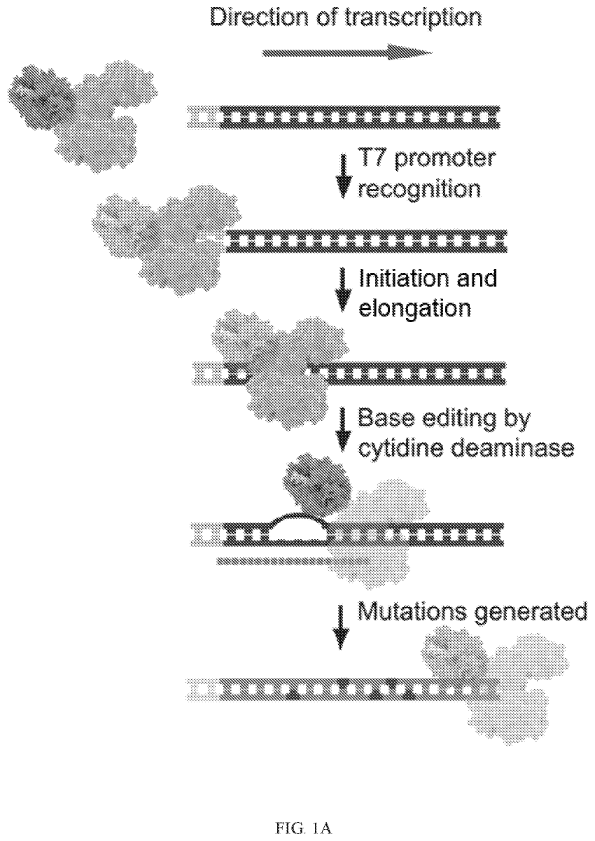

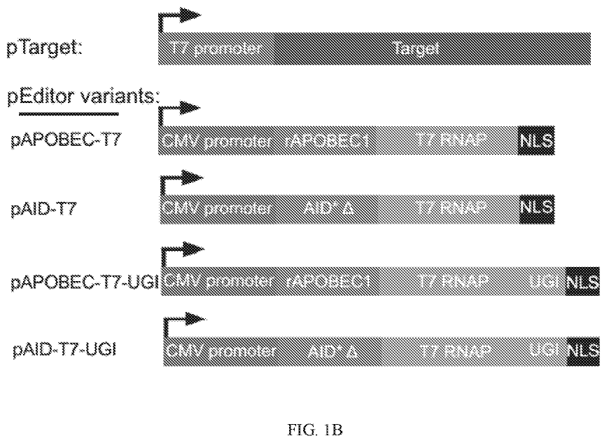

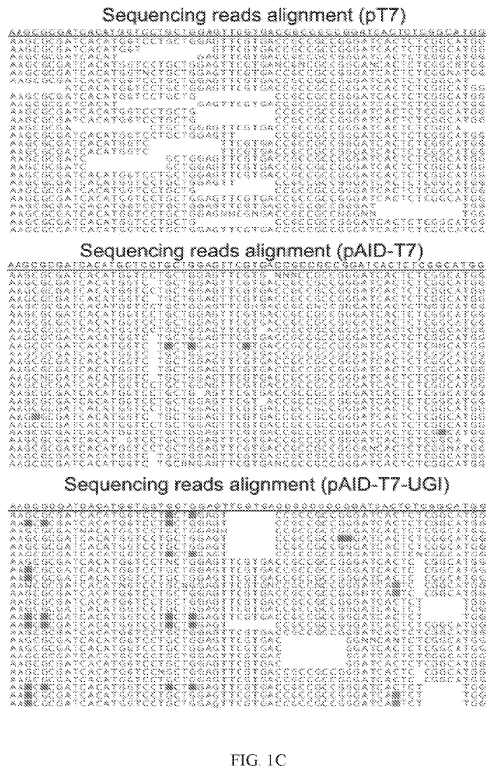

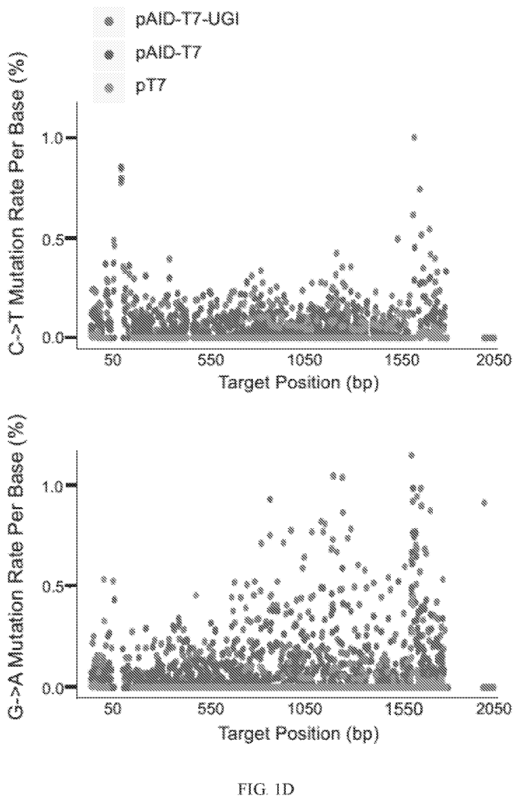

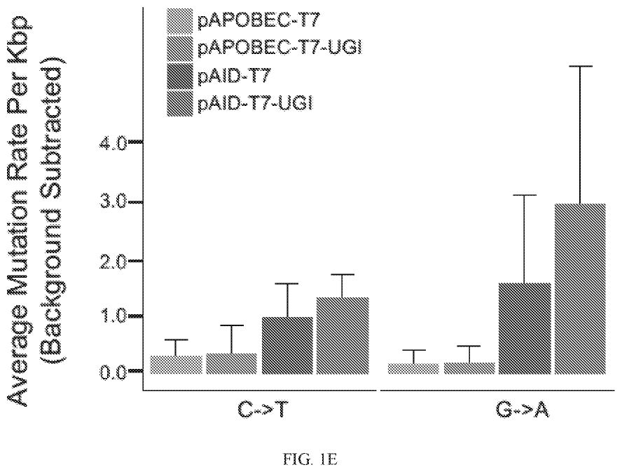

It was initially examined whether combining T7 RNAP with a cytidine deaminase could create a means of continuously diversifying DNA nucleotides downstream of a T7 promoter (FIG. 1A). This was tested by devising a dual-plasmid system (pTarget, pEditor), with pTarget containing an EGFP gene downstream of a T7 promoter and pEditor containing the T7 RNAP-cytidine deaminase fusion gene with a nuclear localization signal (FIG. 1B). Two variants of the cytidine deaminase, rat APOBEC1 and a hyperactive mutant of AID (AID*4), previously selected for their reported strong catalytic activity (4, 11), were selected for pEditor. Additionally, variants containing a uracil DNA glycosylase inhibitor (UGI), which has been shown to facilitate C:G->T:A mutations (11), fused to the 3′ end were also tested (FIG. 1B).

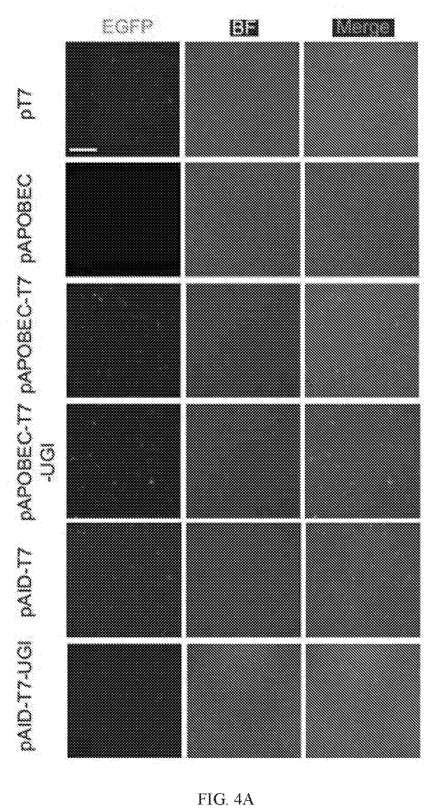

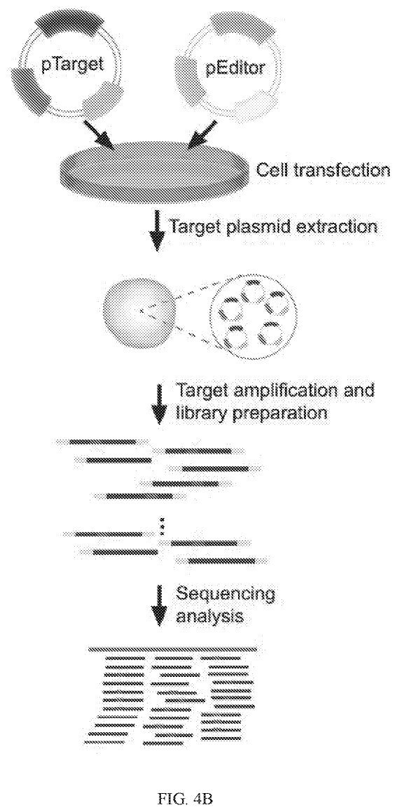

To test whether fusing a cytidine deaminase to T7 RNAP maintained T7 RNAP activity, pTarget and various pEditor plasmids were transfected into HEK 293T cells and EGFP fluorescence under each condition was measured. Consistent with previous reports (9, 10), T7 RNAP alone (pT7) was able to drive EGFP expression, while deaminase alone (pAPOBEC) could not (FIG. 4A). All variants of cytidine deaminase-T7 RNAP fusions induced EGFP expression (FIG. 4A), which indicated that the T7 RNAP-deaminase fusion proteins maintained the transcriptional activity of T7 RNAP.

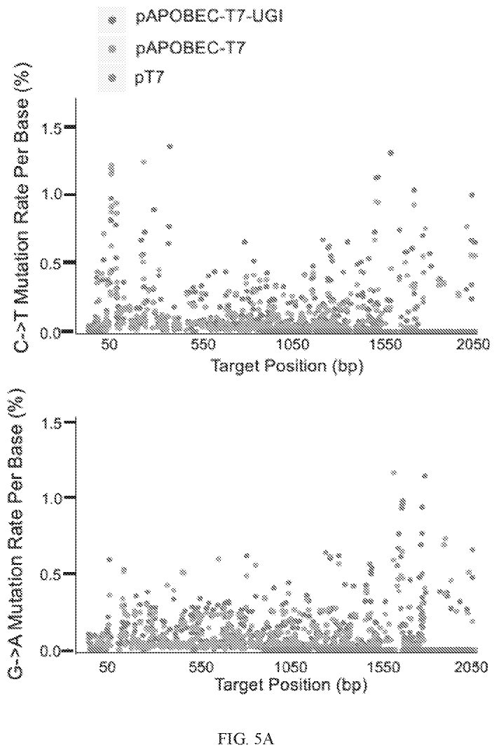

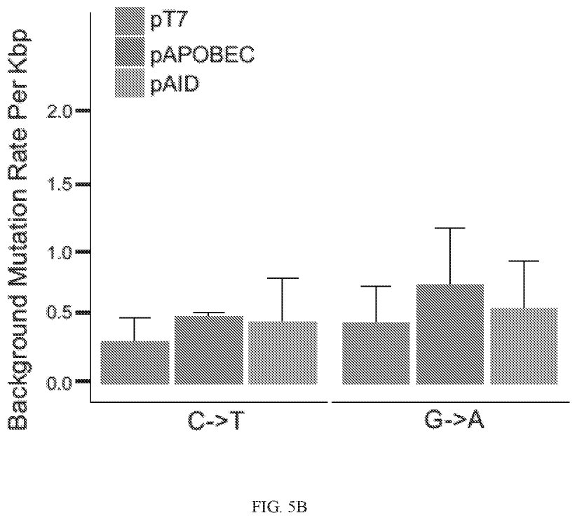

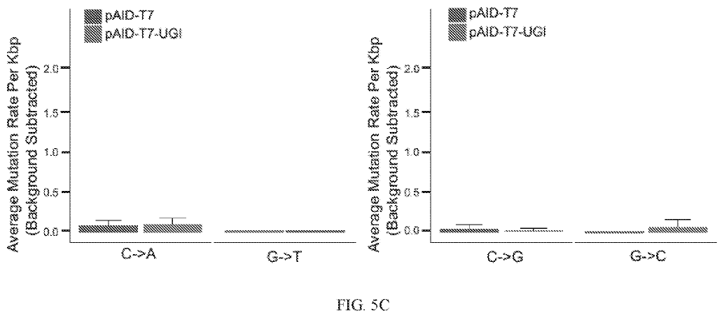

The ability of the T7 RNAP-deaminase fusion protein to induce mutations was then tested within a targeted region. HEK293T cells transfected with both pTarget and pEditor were collected 3 days after transfection. pTarget plasmids were then extracted, and a downstream 2000-bp window was amplified by PCR for high-throughput sequencing (FIG. 5B and Example 1, above). Representative reads from pT7, pAID-T7, and pAID-T7-UGI aligned to the same region within the 2000-bp window are shown in FIG. 1C. Cells transfected with pAID-T7-UGI contained the most number of reads with C->T (green) and G->A (red) mutations, whereas very few reads in the pT7 control group were found to harbor such mutations. It was observed that both C->T and G->A mutation events caused by the cytidine deaminase-T7 RNAP fusion proteins were identified across the entire length of the 2000-bp window, with mutation rates at multiple base positions at ˜0.5-2% (represented as the percentage of reads harboring the mutation at each base; FIG. 1D and FIG. 5A). In contrast, the control pT7 group exhibited mutation rates of less than 0.1% for the majority of bases (which is similar to the error rate expected with Illumina® sequencing chemistry; FIG. 1D and FIG. 5A). Thus, mutation rates in the pT7 group were treated as measurement background (i.e., sequencing errors).

The overall average C->T and G->A mutation rates for each of the pEditor variants was then calculated. The most efficient variant, which was observed to be pAID-T7-UGI, showed an average C->T mutation rate of 1.30 per 1000 base pairs (kbp⁻¹) and an average G->A mutation rate of 2.92 kbp⁻¹(FIG. 1E), which was approximately 500,000-fold higher than the basal somatic mutation frequency in human cells (12). Although not as efficient as the pAID-T7-UGI variant, the pAID-T7 variant was still identified as capable of inducing an average C->T mutation rate of ˜0.97 kbp⁻¹ and an average G->A mutation rate of ˜1.55 kbp⁻¹. The fact that both C->T and G->A substitutions were observed in the data indicated that there was no significant mutational strand bias. The two AID constructs (pAID-T7-UGI and pAID-T7) exhibited higher enzymatic activity than APOBEC constructs, with the pAPOBEC-T7 variant showing an average C->T mutation rate of ˜0.3 kbp⁻¹ and an average G->A mutation rate of ˜0.15 kbp⁻¹, while the pAPOBEC-T7-UGI variant showed an average C->T mutation rate of ˜0.33 kbp⁻¹ and an average G->A mutation rate of ˜0.17 kbp⁻¹ (FIG. 1E). Of note, cells transfected with only cytidine deaminase (pAPOBEC or pAID) showed C->T and G->A mutation rates similar to the background measurement error rates (i.e., similar to that of pT7, (FIG. 5B; pT7 vs. pAPOBEC, two-sided t test, p=0.1201 in C->T, p=0.2244 in G->A; pT7 vs. pAID, two-sided t test, p=0.3625 in C->T, p=0.5877 in G->A), which indicated high specificity of the system. Moreover, although high mutation rates were observed for C->T and G->A base substitutions in AID variants, low mutation rates (<0.1 kbp⁻¹) were observed in other combinations of base substitutions, in line with the primary mutational profile of cytidine deamination (FIG. 5C).

### Example 3: Use of PRIME to Mutate Targeted Gene Loci within the Human Genome

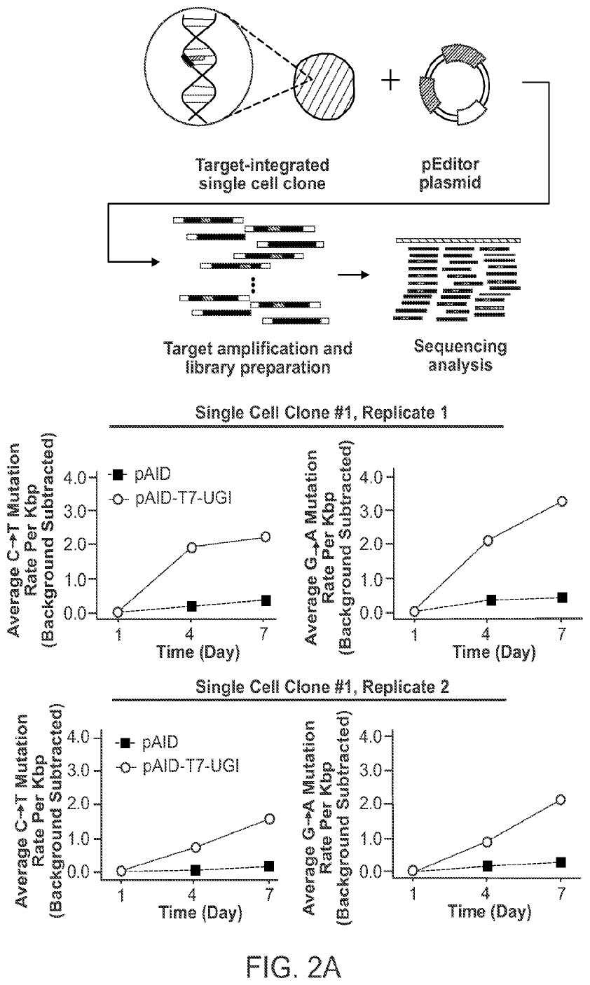

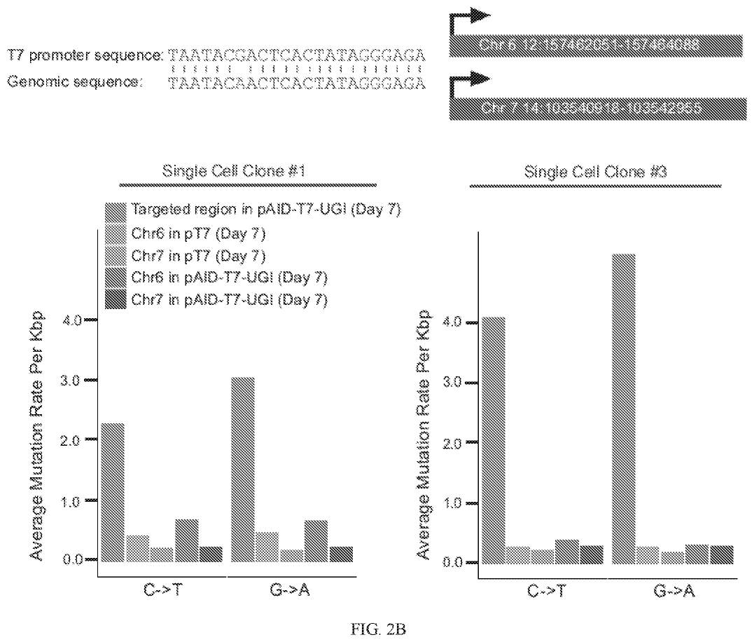

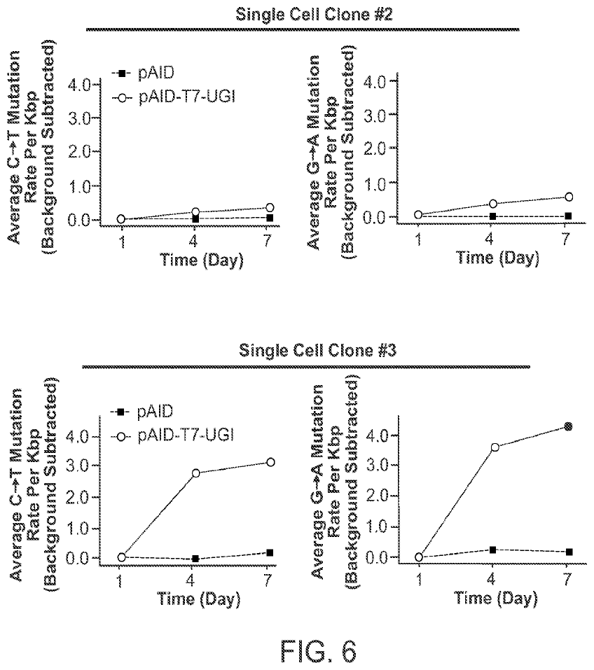

PRIME was then utilized to mutate targeted gene loci within the human genome. An EGFP gene under the control of a T7 promoter was integrated into the HEK293T genome via lentiviral transduction. A CMV promoter was also included upstream of the T7 promoter, to allow for subsequent single cell sorting by EGFP fluorescence. A single cell clone of the EGFP construct-integrated cells was then selected and expanded (FIG. 2A). By transfecting pEditor variant pAID-T7-UGI into the integrated single cell clonal cell line, it was observed to be possible to achieve an average C->T and G->A mutation rate of more than 1-2 kbp⁻¹ three days after transfection (FIG. 2A). Furthermore, another round of pEditor transfection increased the average mutation rate by another 1-2 kbp⁻¹ within the second 3-day period (FIG. 2A). In contrast, no significant accumulation of mutations was observed in the control pAID group at either time point (FIG. 2A). PRIME activity was then examined in an additional two single cell clones. Although it was observed that there were variations in mutation rates across single cell clones in the pAID-T7-UGI group(s), the trend in the accumulation of mutations in the targeted genome region over time remained consistent among all cell clones tested (FIG. 6). The heterogeneity observed was likely due to differences in integration copy number and/or genomic accessibility of the integrated T7 promoter to the PRIME system.

To examine potential off-target effects of the PRIME system in the genome, a search for regions in the genome that possess the conserved T7 promoter sequence (TAATACGACTCACTATAG; SEQ ID NO: 1) was performed. Although an exact match for the T7 promoter sequence in the human genome was not identified, three regions possessing a single-base mismatch, located at distinct locations in chromosomes 6, 7 and 8, respectively, were identified. Among them, the regions in chromosome 6 and 7 (designated “Chr6” and “Chr7”, respectively) shared the same sequence (TAATACAACTCACTATAG; SEQ ID NO: 1) (FIG. 2B, upper panel). The genomic mutation rate of the 2000-bp window immediately after Chr6 and Chr7 was observed using targeted genomic sequencing (see Example 1, above). After 7 days of expression of pAID-T7-UGI, the average C->T and G->A mutation rates of the two regions were observed to be similar to cells expressing pT7 only (˜0.2-0.5 kbp⁻¹), whereas the PRIME-targeted regions (i.e., the regions downstream of the integrated T7 promoter in the genome) showed significant edits (˜2.0-4.5 kbp⁻¹ n=2 biological replicates across 2 single cell clones; FIG. 2B, lower panel). Thus, off-target effects were identified to be minimal/undetectable as compared to background.

### Example 4: Modification of the T7 RNAP Elongation Rate Rendered the Editing Rate of PRIME to be Tunable

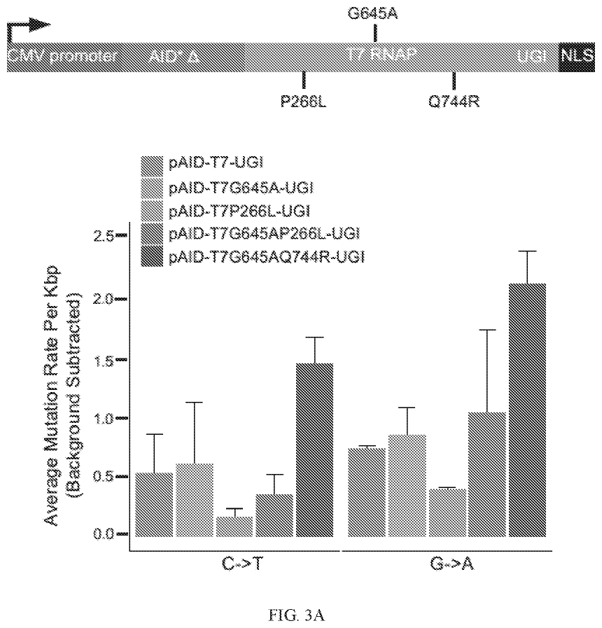

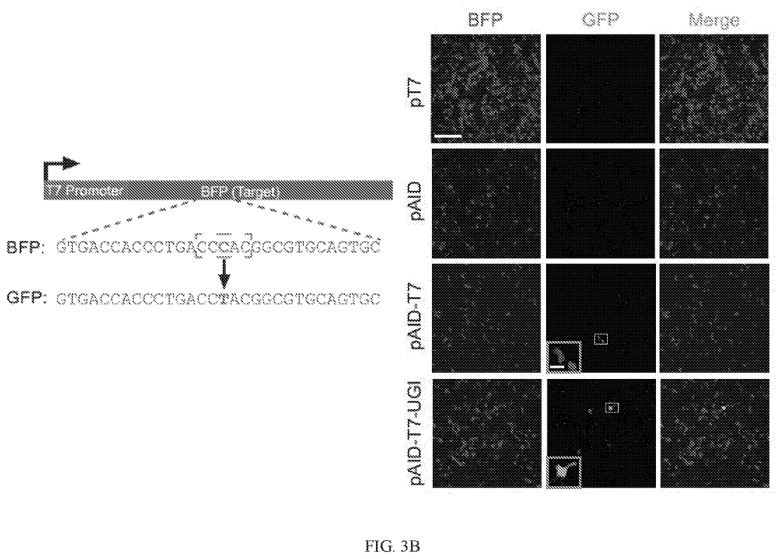

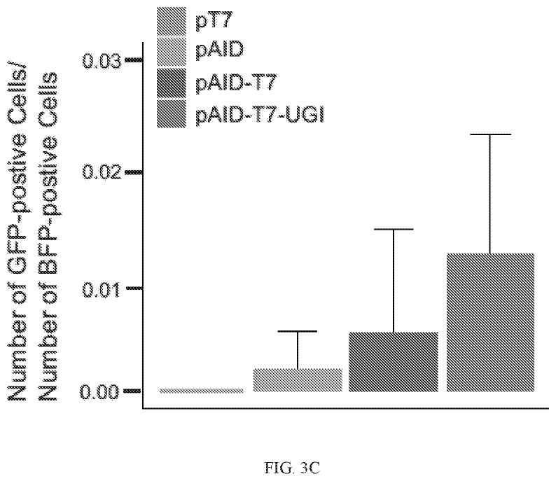

T7 RNAP is widely used in biotechnology and has previously been shown to be highly engineerable. It was examined if the editing rate of PRIME could be tuned by modifying the elongation rate of T7 RNAP or its processivity over the DNA template, as, without wishing to be bound by theory, such changes would be expected to modulate the probability of cytidine deaminase-DNA template interaction. To this end, three mutations (P266L, G645A, Q744R) relative to the wild type T7 RNAP were constructed and tested, with these particular mutations identified based upon previous studies (FIG. 3A, upper panel). P226L was previously shown to enhance the DNA processivity of T7 RNAP over a subregion of the initially transcribed sequence, although this mutation also decreased T7 RNAP affinity for the promoter (13). The G645A mutation was previously shown to decrease the elongation rate of wild type T7 RNAP14, and Q744R was previously shown to enhance the specific activity of the polymerase (15). pEditor variants pAID-T7G645A-UGI, pAID-T7P266L-UGI, pAID-T7P266LG645A-UGI and pAID-T7G645AQ744R-UGI were constructed and compared for their editing efficiency, as compared to pAID-T7-UGI, in a single cell clone integrated with T7 promoter-controlled target. Across two biological replicates, pEditor variant pAID-T7G645AQ744R-UGI induced average C->T and G->A mutation rates that were more than 2-fold higher than those of the wild type pAID-T7-UGI, whereas pAID-T7P266L-UGI reduced the mutation rates by a factor of 2 (FIG. 3A, lower panel).

To demonstrate PRIME can perform functional mutagenesis in mammalian systems, PRIME was used to shift the fluorescence spectra of blue fluorescent protein (BFP). A single H66Y amino acid substitution (in this case, CAC->TAC or TAT) has been previously identified to cause a shift in the fluorescence excitation and emission spectra of BFP, to that of GFP16 (FIG. 3B). The BFP gene was placed under the control of a T7 promoter and a CMV promoter (pBFP), and the pBFP plasmid was introduced alongside pEditor variants into HEK293T cells. After 3 days, fluorescence microscopy and automatic cell counting by Cellprofiler was used to assay the ratio between the number of GFP positive cells and the number of BFP-positive cells. GFP-positive cells were observed in both pAID-T7 (˜0.5%) and pAID-T7-UGI (˜1.2%) groups, whereas spectrum shifts in BFP were not observed in the pT7 group. It was also noted that less than 0.2% of cells in the pAID group became GFP positive (FIG. 3C).

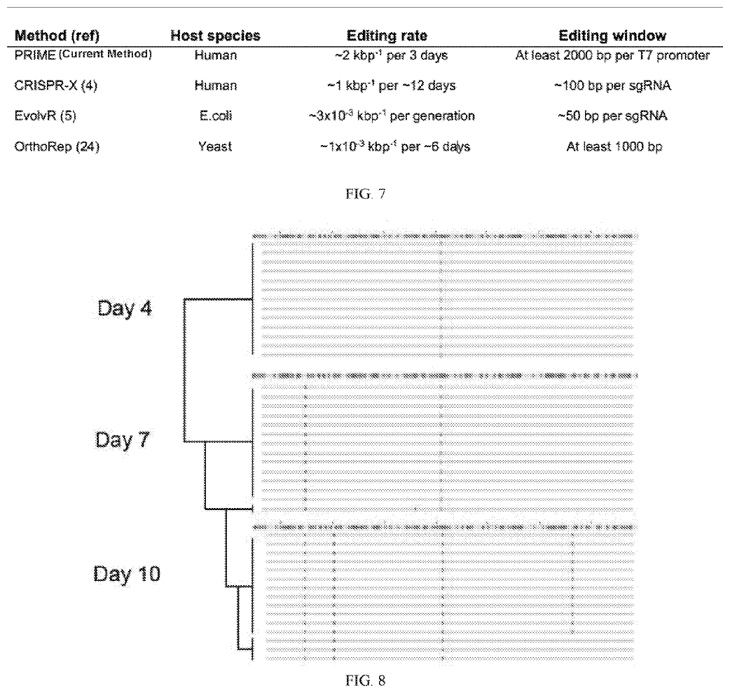

In summary, the above examples have demonstrated that cytidine deaminase fused to T7 RNAP can be used to generate localized nucleotide diversity within the human genome at an average C->T and G->A mutation rate ranging from ˜0.4-4 kbp⁻¹ within a week. Higher editing efficiency may be achieved via additional engineering of the T7 RNAP. The wide editing window of PRIME (>2000 bps) makes it possible to target a long stretch of a selected genomic region over multiple cellular generations. In comparing PRIME with other reported directed evolution methods (FIG. 7), PRIME has demonstrated herein its superiority in terms of both high editing rate and wide editing window. PRIME can be leveraged to evolve both new protein functions and new cellular systems. By introducing T7 promoters to different genes of interest, it is anticipated that this system can simultaneously diversify multiple genomic loci without disrupting reading frames, by avoiding insertions and deletions observed with other DNA editors (17, 18). The base-editing profile of the system can also be greatly expanded by utilizing other base editing enzymes, such as the newly evolved adenine deaminases (19) in concert with cytidine deaminases. Moreover, multiplexed-PRIME systems utilizing orthogonal bacteriophage polymerase systems (e.g., SP6 RNAP) may allow differential editing on multiple loci. Additionally, the highly efficient pseudo-random DNA editing property of PRIME opens doors to a wider range of applications that are not limited to directed evolution. Due to its ubiquity and durability, genomic DNA serves as an ideal medium for recording artificial biological information (20). PRIME is also well suited to serve as a cellular recorder for long-term storage of information using DNA as a medium for the following reasons: 1) PRIME enables continuous targeted mutagenesis in genomic loci over multiple cellular generations, which is a prerequisite for long-term information storage; 2) The toolkit for the PRIME system can be greatly expanded by engineering different editor variants which induce varying targeted mutation rates ranging from ˜0.4-4 per kbp⁻¹ within a week. This gives users flexibility in choosing the one variant that best suits their experimental needs regarding the time-scale of the cellular recording; 3) the wide editing window of PRIME (at least 2000 bps) ensures that the editable sites in the genome will not be exhausted within a short time frame, which is beneficial to applications such as long term lineage tracing and 4) a multiplexed-PRIME system is contemplated as making multi-event analog recording possible. PRIME therefore provides an engineer-able and generalized platform for nucleotide diversification in mammalian systems.

### Example 5: In Vitro and In Vivo Recording of Cell Lineages Using TRACE

TRACE (T7 polymeRAce-driven Continuous Editing), as described herein and also referred to herein as “PRIME”, is a method that enables continuous, targeted mutagenesis in human cells using a cytidine deaminase fused to T7 RNA polymerase. TRACE can be applied to enable cell lineage recordings both in vitro and in vivo. A reconstruction of lineage trees by grouping and ranking DNA mutations from sequencing reads is shown in FIG. 8. In this experiment, a pool of HEK294 cells were sparsely integrated with barcoded lentiviral TRACE templates so that each integrated cell had a unique barcoded TRACE template. Mutation accumulation over time was demonstrated within the same molecular lineage. Reads which shared a unique lentiviral barcode also shared private clonal, and hierarchical sub-clonal mutations which accumulated over time, which demonstrated the usefulness of TRACE for lineage tracing.

A TRACE transgenic mouse is generated by decomposing the TRACE system into two components: the TRACE editor consisting of the T7 RNA-polymerase deaminase fusion protein, and the T7 recording template consisting of a T7 promoter and a transcribed editing template. Both the TRACE editor as well as the T7 promoter-recording template are integrated into a mouse at the Rosa 26 locus. Oocytes containing a T7 promoter-recording template are then fertilized with sperm harboring a constitutively active TRACE editor to initiate sequence diversification in the whole embryo. In addition, to enable cell type-specific lineage tracing, existing mouse lines expressing cell type-specific Cre-recombinase or Cre-ER (a tamoxifen inducible version of Cre) are leveraged to drive the conditional expression of a stably integrated TRACE editor in cells where Cre-recombinase is present. Thus, by crossing the TRACE mouse line with a Cre-driver line, cell-type specific lineage recording is achieved, and additional temporal resolution is provided by tamoxifen induction.

### REFERENCES

1. Farzadfard, F. & Lu, T. K. Emerging applications for DNA writers and molecular recorders. Science 361, 870-875 (2018).
2. Esvelt, K. M., Carlson, J.C. & Liu, D. R. A system for the continuous directed evolution of biomolecules. Nature 472, 499-503 (2011).
3. Su, T. et al. A CRISPR-Cas9 Assisted Non-Homologous End-Joining Strategy for Onestep Engineering of Bacterial Genome. Scientific reports 6, 37895 (2016).
4. Hess, G. T. et al. Directed evolution using dCas9-targeted somatic hypermutation in mammalian cells. Nature methods 13, 1036-1042 (2016).
5. Halperin, S. O. et al. CRISPR-guided DNA polymerases enable diversification of all nucleotides in a tunable window. Nature 560, 248-252 (2018).
6. Moore, C. L., Papa, L. J., 3rd & Shoulders, M. D. A Processive Protein Chimera Introduces Mutations across Defined DNA Regions In Vivo. Journal of the American Chemical Society 140, 11560-11564 (2018).
7. Alexander, D. L. et al. Random mutagenesis by error-prone pol plasmid replication in Escherichia coli. Methods in molecular biology (Clifton, N.J.) 1179, 31-44 (2014).
8. Chamberlin, M., Kingston, R., Gilman, M., Wiggs, J. & deVera, A. Isolation of bacterial and bacteriophage RNA polymerases and their use in synthesis of RNA in vitro. Methods in enzymology 101, 540-568 (1983).
9. Lieber, A., Kiessling, U. & Strauss, M. High level gene expression in mammalian cells by a nuclear T7-phase RNA polymerase. Nucleic acids research 17, 8485-8493 (1989).
10. Ghaderi, M. et al. Construction of an eGFP Expression Plasmid under Control of T7 Promoter and IRES Sequence for Assay of T7 RNA Polymerase Activity in Mammalian Cell Lines. Iranian journal of cancer prevention 7, 137-141 (2014).
11. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
12. Milholland, B. et al. Differences between germline and somatic mutation rates in humans and mice. Nature communications 8, 15183 (2017).
13. Guillerez, J, Lopez, P. J., Proux, F., Launay, H. & Dreyfus, M. A mutation in T7 RNA polymerase that facilitates promoter clearance. Proceedings of the National Academy of Sciences 102, 5958-5963 (2005).
14. Bonner, G., Lafer, E. M. & Sousa, R. Characterization of a set of T7 RNA polymerase active site mutant. The Journal of Biological Chemistry 269, 25120-25128(1994).
15. Boulin, J. C. et al. Mutants with higher stability and specific activity from a single thermosensitive variant of T7 RNA polymerase. Protein Engineering, Design and Selection 26, 725-734 (2013).
16. Glaser, A., McColl, B. & Vadolas, J. GFP to BFP Conversion: A Versatile Assay for the Quantification of CRISPR/Cas9-mediated Genome Editing. Molecular therapy. Nucleic acids 5, e334 (2016).
17. Jakociunas, T., Pedersen, L. E., Lis, A. V., Jensen, M. K. & Keasling, J. D. CasPER, a method for directed evolution in genomic contexts using mutagenesis and CRISPR/Cas9. Metabolic engineering 48, 288-296 (2018).
18. Spanjaard, B. et al. Simultaneous lineage tracing and cell-type identification using CRISPR-Cas9-induced genetic scars. Nature biotechnology 36, 469-473 (2018).
19. Gaudelli, N. M. et al. Programmable base editing of A*T to G*C in genomic DNA without DNA cleavage. Nature 551, 464-471 (2017).
20. Church, G. M., Gao, Y. & Kosuri, S. Next-generation digital information storage in DNA. Science 337, 1628 (2012).
21. Carpenter, A. E. et al. CellProfiler: image analysis software for identifying and quantifying cell phenotypes. Genome Biology 7:R100 (2006).
22. Landini, G, Randell, D. A., Fouad, S, and Galton, A. Automatic thresholding from the gradients of region boundaries. Journal of Microscopy 265, 185-195 (2017).
23. Langmead, B. & Salzberg, S. L. Fast gapped-read alignment with Bowtie 2. Nature methods 9, 357-359 (2012).
24. Ravikumar, A., Arzumanyan, G. A., Obadi, M. K. A. & Liu, C. C. Scalable, continuous evolution of genes at mutation rates above genomic error thresholds. Cell 175, 1-12 (2018).

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A fusion protein comprising:
(i) a bacteriophage RNA polymerase and
(ii) a nucleic acid-editing deaminase.

2. The fusion protein of claim 1, wherein the bacteriophage RNA polymerase is selected from the group consisting of a T7 RNA polymerase and a T7-like RNA polymerase, optionally wherein the T7-like RNA polymerase is a N4 RNA polymerase.

3. The fusion protein of claim 1, wherein the nucleic acid-editing deaminase is selected from the group consisting of a cytidine deaminase, an adenine deaminase and a guanine deaminase, optionally wherein the cytidine deaminase is an activation-induced cytidine deaminase, optionally wherein the activation-induced cytidine deaminase is rat APOBEC1 or AID, optionally wherein the AID cytidine deaminase is a hyperactive mutant of AID, optionally wherein the hyperactive mutant of AID is AID*Δ.

4. The fusion protein of claim 1, further comprising a nuclear localization signal (NLS), optionally wherein the NLS is attached at the C-terminus of the fusion protein.

5. The fusion protein of claim 1, further comprising a uracil glycosylase inhibitor (UGI), optionally wherein the UGI is attached at a location C-terminal to the nucleic acid-editing deaminase and the bacteriophage RNA polymerase.

6. A nucleic acid comprising:
(i) a nucleic acid sequence encoding for a bacteriophage RNA polymerase and
(ii) a nucleic acid sequence encoding for a nucleic acid-editing deaminase.

7. The nucleic acid of claim 6, wherein:
the bacteriophage RNA polymerase is selected from the group consisting of a T7 RNA polymerase and a T7-like RNA polymerase, optionally wherein the T7-like RNA polymerase is a N4 RNA polymerase; and/or
the nucleic acid-editing deaminase is selected from the group consisting of a cytidine deaminase, an adenine deaminase and a guanine deaminase, optionally wherein the cytidine deaminase is an activation-induced cytidine deaminase, optionally wherein the activation-induced cytidine deaminase is rat APOBEC1 or AID, optionally wherein the AID cytidine deaminase is a hyperactive mutant of AID, optionally wherein the hyperactive mutant of AID is AID*Δ.

8. (canceled)

9. The nucleic acid of claim 6, further comprising:
a nucleic acid sequence encoding for a nuclear localization signal (NLS), optionally wherein nucleic acid sequence encoding for the NLS is attached at the 3′-terminus of the nucleic acid;
a nucleic acid sequence encoding for a uracil glycosylase inhibitor (UGI), optionally wherein the nucleic acid sequence encoding for the UGI is attached at a location 3′ of the nucleic acid sequence encoding for the nucleic acid-editing deaminase and the nucleic acid sequence encoding for the bacteriophage RNA polymerase;
a mammalian expression vector promoter, optionally wherein the mammalian expression vector promoter is located 5′ of the nucleic acid sequence encoding for a bacteriophage RNA polymerase and the nucleic acid sequence encoding for the nucleic acid-editing deaminase, optionally wherein the mammalian expression vector promoter is selected from the group consisting of a CMV promoter, a SV-40 promoter, an (EF)-1 promoter and a tetracycline-inducible mammalian promoter; and/or
an origin of replication, optionally wherein the nucleic acid is a plasmid.

10-12. (canceled)

13. A mammalian cell comprising a first nucleic acid of claim 6.

14. The mammalian cell of claim 13, wherein the cell further comprises a second nucleic acid comprising a bacteriophage promoter corresponding to the bacteriophage RNA polymerase of the first nucleic acid, optionally wherein the bacteriophage promoter is a T7 promoter or is a T7-like promoter, optionally wherein the T7-like promoter is a N4 promoter.

15. The mammalian cell of claim 14, wherein:
the bacteriophage promoter of the second nucleic acid is operably linked to a target nucleic acid sequence, optionally wherein the target nucleic acid sequence is a mammalian target nucleic acid sequence, optionally wherein the mammalian target nucleic acid sequence is selected from the group consisting of ABL1, FLT3, MCL1, PRKCQ, WEE1, ABL2, FNTA, MDM2, PRKCSH, XIAP, AKT1, GSK3A, MEK1, PRKCZ, AKT2, GSK3B, MET, PRKDC, AKT3, HDAC1, MTOR, PSENEN, ALK, HDAC2, NFKB1, PSMB5, AR, HDAC3, NTRK1, PTK2, ATM, HDAC6, P4HB, PTPN11, AURKA, HDAC8, p53, PTPN6, AURKB, HER2, PAK1, RAC1, AURKC, HSP90AA1, PARP1, RET, BCL2, HSP90AB1, PDGFRA, ROCK1, BCL ABL1, HSP90AB4P, PDGFRB, ROCK2, BMX, HSP90B1, PDK1, RPS6KA1, BRAF, HSP90B3P, PIK3CA, RPS6KA2, BTK, IGF1R, PIK3CB, RPS6KA3, CASP3, IKBKE, PIK3CD, RPS6KA4, CCR5, ITK, PIK3CG, RPS6KA5, CDK1, JAK2, PLK1, RPS6KA6, CDK2, KDR, PLK2, RPS6KB2, CDK4, KIT, PLK3, RXRA, CDK6, KRAS, PPMID, RXRB, CDK7, MAP2K1, PRKCA1, SGK3, CTNNB1, MAP2K2, PRKCA, SMO, DHFR, MAPK11, PRKCB, SRC, EGFR, MAPK12, PRKCD, SYK, ERBB2, MAPK13, PRKCE, TBK1, FGFR1, MAPK14, PRKCG, TEC, FGFR3, MAPK7, PRKCH, TNF, FLT1, MAPK8, PRKCI and TOP1;
the second nucleic acid is harbored on a plasmid within the mammalian cell;
the second nucleic acid is integrated into the genome of the mammalian cell, optionally wherein the second nucleic acid is integrated into the genome of the mammalian cell at the Rosa 26 locus, optionally wherein the first nucleic acid and the second nucleic acid are integrated into the genome of the mammalian cell at the Rosa 26 locus;
the mammalian cell is a mouse cell, optionally a mouse oocyte cell; and/or
the mammalian cell is a cell of a mammalian cell line, optionally wherein the mammal cell line is selected from the group consisting of HEK293T, VERO, BHK, HeLa, CV1, MDCK, 3T3, a myeloma cell line, PC12, WI38, and Chinese hamster ovary (CHO).

16-18. (canceled)

19. The mammalian cell of claim 15, further comprising a cell type-specific Cre-recombinase or Cre-ER capable of inducing conditional expression of the first nucleic acid and/or the second nucleic acid where Cre-recombinase is present.

20. (canceled)

21. A method for performing mutagenesis upon a target nucleic acid of a mammalian cell, the method comprising:
(a) providing a mammalian cell;
(b) contacting the mammalian cell with:
(i) a first nucleic acid of claim 6; and
(ii) a second nucleic acid comprising a bacteriophage promoter operably linked to a target nucleic acid;
wherein said contacting with said first nucleic acid and said second nucleic acid is performed in any order, including concurrently; and
(c) culturing the mammalian cell for a duration of time sufficient for mutation of the target nucleic acid to be detected.

22. The method of claim 21, wherein the first nucleic acid is harbored on a plasmid, optionally wherein said contacting step (b) comprises transfecting the first nucleic acid into the mammalian cell.

23. (canceled)

24. The method of claim 21, wherein said contacting step (b) comprises genomic integration of the first nucleic acid.

25. The method of claim 21, wherein the second nucleic acid is harbored on a plasmid, optionally wherein said contacting step (b) comprises transfecting the second nucleic acid into the mammalian cell.

26. (canceled)

27. The method of claim 21, wherein said contacting step (b) comprises genomic integration of the second nucleic acid.

28. A kit comprising a nucleic acid of claim 6 and instructions for its use.

29. The kit of claim 28, further comprising a transfection agent, optionally wherein the transfection agent is a lentivirus.
